(19) United States
(12) Patent Application Publication
(10) Pub. No.: US 2024/0240155 A1
(43) Pub. Date: Jul. 18, 2024

(54) Organoid-Derived Monolayers and Uses Thereof

(71) Applicant: Hub Organoids IP B.V., CM Utrecht (NL)

(72) Inventors: Farzin Pourfarzad, Utrecht (NL); Merel Derksen, Utrecht (NL); Alessandra Merenda, Utrecht (NL); Sylvia Fernandez-Boj, Utrecht (NL); Robert Gerhardus Jacob Vries, Utrecht (NL)

(73) Assignee: Hub Organoids IP B.V., CM Utrecht (NL)

(21) Appl. No.: 18/573,478

(22) PCT Filed: Jul. 11, 2022
(86) PCT No.: PCT/EP2022/069354

§ 371 Date: Dec. 22, 2023

(30) Foreign Application Priority Data
Jul. 9, 2021 (EP) 2109913.0

(51) Int. Cl. C12N 5/071; G01N 33/50

(52) U.S. Cl. CPC C12N 5/0679; C12N 5/0686; C12N 5/0688; G01N 33/5082; G01N 33/5091; C12N 2501/105; C12N 2501/11; C12N 2501/115; C12N 2501/12; C12N 2501/999; C12N 2509/10; C12N 2513/00; C12N 2533/90; G01N 2800/52

(57) ABSTRACT

The invention relates to culture methods, in particular methods of obtaining organoid-derived monolayers, and to uses of the organoid-derived monolayers obtained by said methods. The invention also relates to assays for epithelial barrier function and methods of screening compounds using said assays.

All documents cited herein are incorporated by reference in their entirety.

### TECHNICAL FIELD

The invention relates to culture methods, in particular methods of obtaining two-dimensional organoid-derived monolayers. The invention also relates to assays for epithelial barrier function and methods of screening compounds using said assays.

### BACKGROUND

There is great interest in epithelial model systems for cellular assays, drug screening, toxicity assays and the like. Studies on the intestinal epithelium are performed using several in vitro platform systems such as membrane inserts, organs-on-a-chip systems, Ussing chambers, and intestinal rings. These platforms are suitable for establishing polarized epithelial monolayers with access to both apical and basolateral sides of the membrane, using transformed cell lines or primary tissue as models. Although transformed intestinal cell lines, such as the colorectal (adeno)carcinoma cell lines Caco-2, T84, and HT-29, are able to differentiate into polarized intestinal enterocytes or mucus-producing cells to some extent, they are not representative of the in vivo epithelium as several cell types are missing, and various receptors and transporters are aberrantly expressed (Martinez-Maqueda, D., et al. HT29 Cell Line. in The Impact of Food Bio-Actives on Gut Health: In Vitro and Ex Vivo Models. Verhoeckx, K. et al. (eds), Cham (CH): Springer, 113-124 (2015)). In addition, as cell lines are derived from a single donor, they do not represent interpatient heterogeneity and suffer from reduced complexity and physiological relevance. Although primary tissues used in Ussing chambers and as intestinal rings are more representative of the in vivo situation, their limited availability, short-term viability, and lack of expandability make them unsuitable as a medium for high throughput studies.

Accordingly, there is a need for improved in vitro epithelial model systems.

### SUMMARY OF THE INVENTION

The invention provides a method of obtaining an organoid-derived monolayer comprising:
i. digesting or dissociating one or more organoids into a suspension of single cells and/or organoid fragments;
ii. seeding a semi-permeable membrane with said suspension; and
iii. culturing the cells and/or organoid fragments in the presence of an expansion medium until a monolayer is formed.

The invention also provides an organoid-derived monolayer obtainable or obtained by the methods provided herein.

The invention further provides an organoid-derived monolayer which has transepithelial electrical resistance (TEER) of more than 100 Ω·cm².

The invention also provides use of an organoid-derived monolayer of the invention in an assay assessing epithelial viability, metabolic activity, permeability, barrier function integrity and/or activity of transporter proteins.

The invention further provides a method of identifying a compound capable of modulating epithelial viability, metabolic activity, permeability, barrier function integrity and/or activity of transporter proteins comprising:
i. contacting an organoid-derived monolayer, for example according to the invention, with one or more candidate molecules; and
ii. assessing the viability, metabolic activity, permeability and/or barrier function integrity of the organoid-derived monolayer and/or activity of transporter proteins in the organoid-derived monolayer.

The invention further provides a method of assessing the effect of a compound on epithelial viability, metabolic activity, permeability, barrier function integrity and/or activity of transporter proteins comprising:
i. contacting an organoid-derived monolayer, for example according to the invention, with said compound; and
ii. assessing the viability, metabolic activity, permeability and/or barrier function integrity of the organoid-derived monolayer and/or activity of transporter proteins in the organoid-derived monolayer.

The invention further provides a method of identifying a mutation associated with epithelial viability, metabolic activity, permeability, barrier function integrity and/or activity of transporter proteins comprising:
i. assessing the viability, metabolic activity, permeability and/or barrier function integrity of an organoid-derived monolayer and/or activity of transporter proteins in an organoid-derived monolayer, for example an organoid monolayer according to the invention; and
ii. determining the presence of one or more mutations in the genome of one or more cells in the organoid-derived monolayer.

The invention further provides a method of diagnosing a disease or affliction that affects epithelial viability, metabolic activity, permeability, barrier function integrity and/or activity of transporter proteins, or determining an increased risk of said disease or affliction, in a human subject comprising:
i. obtaining an organoid-derived monolayer from said human subject using a method of the invention; and
ii. testing the viability, metabolic activity, permeability and/or barrier function integrity of the organoid-derived monolayer and/or activity of transporter proteins in the organoid-derived monolayer,
   wherein a test result above or below a reference value indicates the presence of, or an increased risk of, said disease or affliction in the human subject.

The invention further provides a method of predicting the likelihood of a patient's response to a candidate compound comprising:
i. obtaining an organoid-derived monolayer from said patient using a method of the invention;
ii. contacting the organoid-derived monolayer with said compound; and
iii. assessing the viability, metabolic activity, permeability and/or barrier function integrity of the organoid-derived monolayer and/or activity of transporter proteins in the organoid-derived monolayer.

### DETAILED DESCRIPTION

### Organoid-Derived Monolayer Preparation and Culture

### Organoid-Derived Monolayer Preparation

Organoid-derived monolayers of the invention are prepared using organoids. “Organoid” refers to a cellular structure obtained by expansion of adult (post-embryonic) epithelial stem cells, preferably characterized by Lgr5 expression, and consisting of tissue-specific cell types that self-organize through cell sorting and spatially restricted lineage commitment (e.g. as described in Clevers, Cell. 2016 Jun. 16; 165(7):1586-1597, see particularly section called “Organoids derived from adult stem cells” at page 1590 onwards). Methods of obtaining and culturing organoids from a variety of tissues have previously been described in WO2010/090513, WO2012/014076, WO2015/173425, WO2016/083613 and WO2017/220586. Preferably, the adult epithelial stem cells are not derived from induced pluripotent stem (iPS) cells.

In some embodiments, an organoid-derived monolayer of the invention is obtained by a method comprising:

digesting or dissociating one or more organoids into a suspension of single cells and/or organoid fragments;

seeding a semi-permeable membrane with said suspension; and

culturing the cells and/or organoid fragments in the presence of an expansion medium until a monolayer is formed.

Organoid fragments include any fragment from an organoid, for example an organoid-derived intestinal crypt. In some embodiments, organoid fragments are cell clumps, preferably consisting of less than 10, less than 5, preferably 2-4 cells. In preferred embodiments, the one or more organoids are digested or dissociated into a suspension comprising single cells and cell clumps.

In some embodiments, following the digestion or dissociation of the one or more organoids, the suspension of single cells and/or organoid fragments is centrifuged and resuspended before seeding the semi-permeable membrane. In some embodiments, the suspension of single cells and/or organoid fragments is adjusted to an appropriate cell density for seeding, for example, to about 0.5×10⁶ cells per mL, about 10⁶ cells per mL, about 2×10⁶ cells per mL, about 3×10⁶ cells per mL, about 4×10⁶ cells per mL or about 5×10⁶ cells per mL. In other embodiments, the suspension of single cells and/or organoid fragments is adjusted to about 0.2×10⁶ cells per mL, about 0.3×10⁶ cells per mL, about 0.4×10⁶ cells per mL, about 0.5×10⁶ cells per mL, about 10⁶ cells per mL, about 2×10⁶ cells per mL, about 3×10⁶ cells per mL, about 4×10⁶ cells per mL or about 5×10⁶ cells per mL. In some embodiments, the suspension of single cells and/or organoid fragments is adjusted to less than about 0.2×10⁶ cells per mL, less than about 0.3×10⁶ cells per mL, less than about 0.4×10⁶ cells per mL, less than about 0.5×10⁶ cells per mL, less than about 10⁶ cells per mL, less than about 2×10⁶ cells per mL, less than about 3×10⁶ cells per mL, less than about 4×10⁶ cells per mL or less than about 5×10⁶ cells per mL before seeding. In some embodiments, the suspension of single cells and/or organoid fragments is adjusted to about 0.1-1×10⁶ cells per mL, about 0.25-0.75×10⁶ cells per mL, about 0.3-0.5×10⁶ cells per mL, about 0.35-0.45×10⁶ cells per mL, preferably about 0.4×10⁶ cells per mL before seeding. In some embodiments, the suspension of single cells and/or organoid fragments is adjusted to less than about 0.5×10⁶ cells per mL, less than about 0.6×10⁶ cells per mL, less than about 0.7×10⁶ cells per mL, less than about 0.8×10⁶ cells per mL, less than about 0.9×10⁶ cells per mL, less than about 10⁶ cells per mL, less than about 1.1×10⁶ cells per mL, less than about 1.2×10⁶ cells per mL, less than about 1.3×10⁶ cells per mL, less than about 1.4×10⁶ cells per mL, less than about 1.5×10⁶ cells per mL before seeding. In some embodiments, the suspension of single cells and/or organoid fragments is adjusted to about 0.2×10⁶ cells per mL, about 0.3×10⁶ cells per mL, about 0.4×10⁶ cells per mL, about 0.5×10⁶ cells per mL, about 10⁶ cells per mL, about 1.5×10⁶ cells per mL, about 2×10⁶ cells per mL, about 3×10⁶ cells per mL, about 4×10⁶ cells per mL or about 5×10⁶ cells per mL. In some embodiments, the suspension of single cells and/or organoid fragments is adjusted to about 0.1-5×10⁶ cells per mL, about 0.25-2.5×10⁶ cells per mL, about 0.5-1.5×10⁶ cells per mL, about 0.75-1.25×10⁶ cells per mL, about 0.8-1.2×10⁶ cells per mL.

As shown in the examples, the seeding density for intestinal cells may preferably be about 0.45×10⁶ cells per mL, the seeding density for lung cells may preferably be about 0.4×10⁶ cells per mL, and the seeding density for kidney cells may preferably be about 10⁶ cells per mL.

In some embodiments, about 0.1×10⁶ cells, about 0.2×10⁶ cells, about 0.3×10⁶ cells, about 0.4×10⁶ cells, about 0.5×10⁶ cells, about 0.6×10⁶ cells, about 0.7×10⁶ cells, about 0.7×10⁶ cells, about 0.8×10⁶ cells, about 0.9×10⁶ cells or about 10⁶ cells are seeded onto a semi-permeable membrane. In some embodiments, about 0.45×10⁶ cells are seeded onto a semi-permeable membrane, for example, in a standard 96-well plate. As shown in the examples, this seeding density is particularly suitable for organoids derived from the intestine.

In some embodiments, less than about 20,000 cells, less than about 30,000 cells, less than about 40,000 cells, less than about 50,000 cells, less than about 60,000 cells, less than about 70,000 cells, less than about 80,000 cells, less than about 90,000 cells, less than about 100,000 cells, or less than about 250,000 cells are seeded onto a semi-permeable membrane. In some embodiments, about 30,000 cells, about 40,000 cells, about 50,000 cells, about 60,000 cells, about 70,000 cells, about 80,000 cells or about 90,000 cells are seeded onto a semi-permeable membrane. In some embodiments, about 5,000-500,000 cells, about 10,000-250,000 cells, about 20,000-100,000 cells, about 30,000-50,000 cells, about 35,000-45,000 cells are seeded onto a semi-permeable membrane. In some embodiments, about 40,000 cells are seeded onto a semi-permeable membrane, for example, in a standard 96-well plate. The inventors have unexpectedly found that seeding a lower number of cells results in higher TEER values in lung monolayers. Accordingly, in some embodiments, particularly where the organoids are derived from the lung, about 40,000 cells are seeded onto a semi-permeable membrane, for example, in a standard 96-well plate.

In some embodiments less than about 100,000 cells, less than about 150,000 cells, less than about 200,000 cells, or less than about 250,000 cells are seeded onto a semi-permeable membrane. In some embodiments, about 30,000 cells, about 40,000 cells, about 50,000 cells, about 60,000 cells, about 70,000 cells, about 80,000 cells, about 90,000 cells, or about 100,000 cells are seeded onto a semi-permeable membrane. In some embodiments, about 20,000-500,000 cells, about 30,000-400,000 cells, about 40,000-300,000 cells, about 50,000-250,000 cells, about 60,000-200,000 cells, about 70,000-150,000 cells, about 80,000-120,000 cells are seeded onto a semi-permeable membrane. In some embodiments, about 100,000 cells are seeded onto a semi-permeable membrane, for example, in a standard 96-well plate. The inventors have unexpectedly found that, whilst seeding a higher number of cells results in higher TEER values in kidney monolayers, this effect plateaus at about 100,000 cells per well in a 96-well plate. Accordingly, in some embodiments, particularly where the organoids are derived from the kidney, about 100,000 cells are seeded onto a semi-permeable membrane, for example, in a standard 96-well plate.

The expansion medium may be any suitable expansion medium for epithelial stem or progenitor cells, preferably a suitable expansion medium for epithelial stem cells (e.g. as described in WO2010/090513, WO2012/014076, WO2012/168930 or WO2015/173425).

In some embodiments, the expansion medium comprises a receptor tyrosine kinase ligand, a BMP inhibitor and a Wnt agonist.

For example, in some embodiments, the expansion medium comprises EGF, Noggin and Wnt-conditioned medium. In some embodiments, the expansion medium comprises EGF, Noggin, Rspondin and Wnt surrogate.

In some embodiments, the expansion medium further comprises nicotinamide and a p38 inhibitor, such as SB202190. In some embodiments, the expansion medium further comprises a TGF-beta inhibitor.

In a preferred embodiment, the expansion medium comprises (i) EGF (e.g. at a concentration of about 50 ng/ml); (ii) Noggin (e.g. at a concentration of about 100 ng/ml); (iii) Rspondin (e.g. at a concentration of about 250 ng/mL); (iv) Wnt surrogate (e.g. NGS-Wnt at a concentration of about 0.5 nM); (v) a p38 inhibitor (e.g. SB-203580 at a concentration of about 10 μM); (vi) a TGF-beta inhibitor (e.g. A83-01 at a concentration of about 500 nM); and (vii) nicotinamide (e.g. at a concentration of about 10 mM).

In another preferred embodiment, the expansion medium comprises (i) EGF (e.g. at a concentration of about 50 ng/ml); (ii) Noggin (e.g. at a concentration of about 100 ng/ml); (iii) Wnt-conditioned medium (e.g. at about 50% final volume); (iv) a p38 inhibitor (e.g. SB-203580 at a concentration of about 10 PM); (v) a TGF-beta inhibitor (e.g. A83-01 at a concentration of about 500 nM); and (vi) nicotinamide (e.g. at a concentration of about 10 mM).

In some embodiments, particularly where the organoids are derived from the lung, the expansion medium comprises one or more receptor tyrosine ligands, a Wnt agonist, a TGF-beta inhibitor and a BMP inhibitor. In some embodiments, the expansion medium comprises FGF, Rspondin, a TGF-beta inhibitor, a BMP inhibitor, a Rho-kinase inhibitor and a p38 inhibitor. In a preferred embodiment, the expansion medium comprises i) FGF (e.g. FGF-7 at a concentration of about 25 ng/ml and FGF-10 at a concentration of about 100 ng/mL); (ii) Rspondin (e.g. Rspondin-3 at a concentration of about 250 ng/mL); (iii) a TGF-beta inhibitor (e.g. A83-01 at a concentration of about 500 nM); (iv) a BMP inhibitor (e.g. Noggin-Fc Fusion Protein conditioned medium at about 2% final volume), (v) a Rho-kinase inhibitor (e.g. Y-27632 at a concentration of about 10 μM), and (vi) a p38 kinase inhibitor (e.g. SB202190 at a concentration of about 500 nM).

In some embodiments, particularly where the organoids are derived from the kidney, the expansion medium comprises one or more receptor tyrosine ligands, a Wnt agonist, and a TGF-beta inhibitor. In some embodiments, the expansion medium comprises EGF, FGF, Rspondin, a TGF-beta inhibitor and a Rho-kinase inhibitor. In a preferred embodiment, the expansion medium comprises i) EGF (e.g. at a concentration of about 50 ng/ml); (ii) FGF (e.g. FGF-10 at a concentration of about 100 ng/ml); (iii) Rspondin (e.g. Rspol-conditioned medium at about 10% final volume); (iv) a TGF-beta inhibitor (e.g. A83-01 at a concentration of about 500 nM); and (v) a Rho-kinase inhibitor (e.g. Y-27632 at a concentration of about 10 μM).

The inventors have shown that, during the step of culturing the cells and/or organoid fragments in the presence of an expansion medium, TEER of the monolayer increases over time and reaches a stable value when the monolayer reaches confluence. In some embodiments, the monolayer is cultured in the presence of an expansion medium until TEER of the monolayer becomes stable, for example, TEER does not increase or decrease by more than 50%, more than 40%, more than 30%, more than 20% or more than 10% in the space of 24 hours, 2 days, 3 days, 4 days, 5 days or more. For example, TEER does not increase or decrease by more than 20% in the space of 24 hours. In some embodiments, the monolayer is cultured in the presence of an expansion medium until it reaches TEER of about 20, about 30, about 40, about 50, about 60, about 70, about 80, about 90 or about 100, about 200, about 300, about 400, about 500, about 600, about 700, about 800, about 900 or about 1000 Ω·cm². In some embodiments, the monolayer is cultured in the presence of an expansion medium until it reaches TEER of about 100 Ω·cm².

The inventors have shown that organoid-derived intestinal monolayers which are further cultured in the presence of a differentiation medium are improved because they achieve a higher TEER than monolayers which have only been cultured in the presence of an expansion medium. A higher TEER translates to a larger dynamic range, making such organoid-derived monolayers particularly useful in assays, for example, to assess epithelial viability, metabolic activity, permeability, barrier function integrity and/or activity of transporter proteins. Accordingly, in preferred embodiments, the method further comprises culturing the monolayer in the presence of a differentiation medium. In some embodiments, the TEER of the monolayer further increases during the step of culturing the monolayer in the presence of a differentiation medium. In some embodiments, TEER of the monolayer reaches more than 500, more than 600, more than 700, more than 800, more than 900, more than 1000, more than 1100, more than 1200, more than 1300, more than 1400 or more than 1500 Ω·cm² during the step of culturing the monolayer in the presence of a differentiation medium.

The differentiation medium may be any suitable differentiation medium for organoids, e.g. as described in WO2015/173425, WO2017/149025 and WO2017/220586. Exemplary differentiation media which may be used with the invention are described herein. A desired cellular composition in the organoid-derived monolayer can be achieved by selecting an appropriate differentiation medium.

The inventors have shown that culturing organoid-derived monolayers in a differentiation medium which comprises a Notch inhibitor, an EGFR pathway inhibitor and a Wnt agonist results in monolayers which have higher TEER values and a more heterogeneous cellular composition than those achieved with other differentiation media.

Accordingly, in a preferred embodiment, the method further comprises culturing the monolayer in the presence of a differentiation medium which comprises a Notch inhibitor, an EGFR pathway inhibitor and a Wnt agonist. In some embodiments, the method comprises culturing the monolayer in the presence of a differentiation medium which comprises a Notch inhibitor, an EGFR pathway inhibitor and a Wnt agonist until the monolayer reaches TEER of at least 500 Ω·cm². In some embodiments, the method comprises culturing the monolayer in the presence of a differentiation medium which comprises a Notch inhibitor, an EGFR pathway inhibitor and a Wnt agonist until the monolayer reaches TEER of at least 1000 Ω·cm². In some embodiments, the method comprises culturing the monolayer in the presence of a differentiation medium which comprises a Notch inhibitor, an EGFR pathway inhibitor and a Wnt agonist until the monolayer reaches TEER of at least 1500 Ω·cm². Exemplary differentiation media which comprise a Notch inhibitor, an EGFR pathway inhibitor and a Wnt agonist are described herein.

The present application exemplifies preparation and use of organoid-derived monolayers which are derived from the intestine, lung and kidney. The intestine is an epithelial tissue, and part of the digestive system. The lung and kidney are also epithelial tissues. Accordingly, the skilled person would appreciate that the methods and use described herein can be applied to other epithelial tissues, particularly other epithelial tissues from the digestive system.

In some embodiments, the organoid-derived monolayer is derived from the digestive system. In some embodiments, the organoid-derived monolayer is derived from the gastrointestinal tract. Preferably, the organoid-derived monolayer is derived from the intestine.

In some other preferred embodiments, the organoid-derived monolayer is derived from the lung.

In yet other preferred embodiments, the organoid-derived monolayer is derived from the kidney.

In some embodiments, the organoid-derived monolayer is derived from a mammal, for example, human, non-human primate, rat, dog or minipig. In some embodiments, the monolayer is derived from a dog. In some embodiments, the monolayer is derived from a rat. Preferably, the monolayer is derived from a human.

In some embodiments, the organoid-derived monolayer is derived from a healthy human subject. In some embodiments, particularly where the organoid-derived monolayer is derived from the digestive system, the organoid-derived monolayer is derived from a human with a disease or disorder of the digestive system, such as inflammatory bowel disease (e.g. Crohn's disease (CD) or ulcerative colitis (UC)), coeliac disease or leaky gut syndrome.

### Wnt Agonists

The expansion medium of the invention comprises a Wnt agonist. In some embodiments, the differentiation medium of the invention comprises a Wnt agonist.

The Wnt signalling pathway and small molecules which activate Wnt signalling are described in Nusse and Clevers (2017, Cell 169(6):985-999). The Wnt signalling pathway when activated typically prevents β-catenin degradation and enhances β-catenin-mediated signalling. This pathway is defined by a series of events that occur when the cell-surface Wnt receptor complex, comprising a Frizzled receptor and LRP5/6 is activated, usually by an extracellular signalling molecule, such as a member of the Wnt family. This results in the activation of Dishevelled family proteins which inhibit a destruction complex of proteins that degrades intracellular β-catenin. The destruction complex is formed of structural components including APC and axin, to which casein kinases CK1α, δ and ε and GSK-3 are recruited. The destruction complex is thought to phosphorylate β-catenin and to expose it to a ubiquitin ligase, β-TrCP. Ubiquitination of the β-catenin then results in its degradation in the proteasome.

The main effector function of β-catenin is in the nucleus, where it regulates transcription through interaction with various transcription factors, including the TCF/LEF family transcription factors (e.g. Tcf-1, Tcf-3, Tcf-4 and Lefl).

The Wnt pathway is highly regulated. For instance, Wnt signalling is enhanced when Rspondin binds to its receptors (Lgr4, Lgr5 and/or Lgr6). However, two transmembrane E3 ubiquitin ligases, Rnf43 and Znrf3, have been shown to remove Rspondin receptors (e.g. Lgr4, Lgr5 and/or Lgr6) from the cell surface (see, e.g., de Lau et al. 2016). Rspondins are vertebrate-specific Wnt-enhancing agents. In addition, the binding of Dishevelled family proteins to the Frizzled receptor can be inhibited by Dapper family proteins (e.g. Dapper1 and Dapper3). Furthermore, the activity of the destruction complex is thought to be partly regulated by the phosphorylation status of APC, axin and GSK-3. For example, dephosphorylation of APC or axin by phosphatases (e.g. serine/threonine phosphatases such as PP1, PP2C or PP2A) may inhibit β-catenin degradation. In addition, phosphorylation of GSK-3 by kinases (e.g. p38 MAPK, PKA, PKB, PKC, p90RSK or p70S6K) may inhibit GSK-3 activity and so inhibit β-catenin degradation.

The stability of the destruction complex is thought to be partly regulated by two PARPs, Tankyrases 1 and 2. Poly(ADP-ribosyl)ation of axin and auto-poly(ADP-ribosyl)ation by these Tankyrases may promote deoligomerisation of the destruction complex.

In the nucleus, Dishevelled family proteins can form a complex with the histone deacetylase SIRT1, which supports the transcription of Wnt target genes.

A protein that is thought to be key to the secretion of Wnt is the multipass membrane protein Porcupine (Porc), the loss of which results in Wnt accumulating in the endoplasmic reticulum.

A Wnt agonist is defined as an agent that activates TCF/LEF-mediated transcription in a cell. Wnt agonists are therefore selected from true Wnt agonists that bind and activate the Wnt receptor complex including any and all of the Wnt family proteins, an inhibitor of intracellular β-catenin degradation, a GSK inhibitor (such as CHIR9901) and activators of TCF/LEF. In some embodiments, a Wnt agonist is a secreted glycoprotein including Wnt-1/Int-1, Wnt-2/Irp (InM-related Protein), Wnt-2b/13, Wnt-3/Int-4, Wnt-3a (R&D systems), Wnt-4, Wnt-5a, Wnt-5b, Wnt-6 (Kirikoshi H et al 2001 Biochem Biophys Res Com 283 798-805), Wnt-7a (R&D systems), Wnt-7b, Wnt-8a/8d, Wnt-8b, Wnt-9a/14, Wnt-9b/14b/15, Wnt-10a, Wnt-10b/12, WnM 1, and Wnt-16. An overview of human Wnt proteins is provided in “THE WNT FAMILY OF SECRETED PROTEINS”, R&D Systems Catalog, 2004. In some embodiments, the Wnt agonist is an inhibitor of RNF43 or ZNRF3. It has been shown that RNF43 and ZNRF3 reside in the cell membrane and negatively regulate levels of the Wnt receptor complex in the membrane, probably by ubiquitination of Frizzled. Therefore, the inventors hypothesise that inhibition of RNF43 or ZNRF3 with antagonistic antibodies, RNAi or small molecule inhibitors would indirectly stimulate the Wnt pathway. RNF43 and ZNRF3 have a catalytic ring domain (with ubiquitination activity), which can be targeted in small molecule inhibitor design. Several anti-RNF43 antibodies and several anti-ZNRF3 antibodies are available commercially. In some embodiments, such antibodies are suitable Wnt agonists in the context of the invention.

The Wnt agonist in the expansion or differentiation medium is preferably any agonist able to stimulate the Wnt pathway via the Lgr5 cell surface receptor, i.e. in a preferred embodiment, the Wnt agonist in the expansion medium is an Lgr5 agonist. Known Lgr5 agonists include Rspondin, fragments and derivatives thereof, and anti-Lgr5 antibodies (e.g. see WO 2012/140274, in particular FIGS. 22-24, and De Lau, W. et al. Nature, 2011 Jul. 4; 476(7360):293-7). A preferred Lgr5 agonist is Rspondin. Any suitable Rspondin may be used, for example, it may be selected from one or more of Rspondin 1, Rspondin 2, Rspondin 3 and Rspondin 4 or derivatives thereof. For example, any of Rspondin 1 (NU206, Nuvelo, San Carlos, CA), Rspondin 2 ((R&D systems), Rspondin 3, and Rspondin-4) may be used. Any suitable concentration of Rspondin may be used, for example, at least 100 ng/ml, more preferred at least 200 ng/ml, more preferred about 250 ng/ml. An example of an agonistic anti-Lgr5 antibody is 1D9 (available commercially from BD Biosciences, BDB562733, No.:562733). Fragments of Rspondin may be used as the Wnt agonist. For example, in some embodiments the Wnt agonist is a fragment of Rspondin comprising or consisting of the furin domain.

In some embodiments, the Wnt agonist in the expansion or differentiation medium is a Wnt surrogate. Wnt surrogate is a water-soluble Wnt agonist engineered by linking antagonistic Fzd and Lrp5/6-binding modules into a single polypeptide chain, thus forcing receptor heterodimerisation while blocking endogenous Wnt binding. Wnt surrogate supports the growth of a broad range of cultures. Furthermore, Wnt surrogate is a non-lipidated Wnt agonists that can be produced in serum free medium, kept frozen and circumvent the differences in activity of Wnt-conditioned media produced by different laboratories (Janda C Y, et al. Surrogate Wnt agonists that phenocopy canonical Wnt and β-catenin signalling. Nature. 2017 May 11; 545(7653):234-237). In some embodiments, the Wnt surrogate is next-generation surrogate Wnt (NGS-Wnt), for example as described in Miao, Y. et al. (Next-generation surrogate Wnts support organoid growth and deconvolute Frizzled pleiotropy in vivo. Cell Stem Cell. 27 (5), 840-851 (2020)). NGS-Wnt may be provided at a concentration of about 0.1 nM to about 0.5 nM. In some embodiments, the expansion medium comprises NGS-Wnt at a concentration of about 0.5 nM. In some embodiments, the differentiation medium comprises NGS-Wnt at a concentration of about 0.1 nM.

The Wnt agonist is preferably added to the media in an amount effective to stimulate a Wnt activity in a cell by at least 10%, more preferred at least 20%, more preferred at least 30%, more preferred at least 50%, more preferred at least 70%, more preferred at least 90%, more preferred at least 100%, relative to a level of said Wnt activity in the absence of said molecule, as assessed in the same cell type. As is known to a skilled person, Wnt activity can be determined by measuring the transcriptional activity of Wnt, for example by pTOPFLASH and pFOPFLASH Tcf luciferase reporter constructs (Korinek et al., 1997. Science 275:1784-1787). Wnt activity may also be determined using LEADING LIGHT® Wnt Reporter Assay Starter Kit (Enzo Life Sciences, cat. no. ENZ-61001-0001), which uses an engineered 3T3 mouse fibroblast cell line expressing the firefly luciferase reporter gene under the control of Wnt-responsive promoters (TCF/LEF).

A soluble Wnt agonist, such as Wnt-3a, may be provided in the form of Wnt conditioned media. For example, about 10% to about 50% Wnt conditioned media may be used.

Rspondin may be provided in the form of Rspo conditioned media. For example, about 10% to about 30%, e.g. about 10 ng/ml to about 10 μg/ml, preferably about 1 μg/ml, Rspo conditioned media may be used.

Examples of Rspondin mimics suitable for use in the invention are provided in WO 2012/140274, which is incorporated herein by reference.

One or more, for example, 2, 3, 4 or more Wnt agonists may be used in the expansion or differentiation medium. In one embodiment, the medium comprises an Lgr5 agonist, for example Rspondin, and additionally comprises a further Wnt agonist. In this context, the further Wnt agonist may, for example, be selected from the group consisting of Wnt-3a, a GSK-inhibitor (such as CHIR99021), Wnt-5, Wnt-6a Norrin, and NGS-Wnt. In one embodiment, the expansion or differentiation medium comprises Rspondin and additionally comprises a soluble Wnt ligand, such as Wnt3a or NGS-Wnt. Addition of a soluble Wnt ligand has been shown to be particularly advantageous for expansion of human epithelial stem cells (as described in WO2012/168930).

### Wnt Inhibitors

In some embodiments, the differentiation medium of the invention comprises a Wnt inhibitor. Any suitable Wnt inhibitor may be used.

The Wnt signalling pathway can be inhibited at many levels and Wnt inhibitors are reviewed in detail in Voronkov and Krauss (2013) Current Pharmaceutical Design 19:634-664, and in Tran and Zheng (2017) Protein Science 26:650-661.Wnt inhibitors are commercially available, e.g. from R&D systems, Santa Cruz Biotechnology and Selleckchem.

A Wnt inhibitor is defined as an agent that inhibits TCF/LEF-mediated transcription in a cell or in a population of cells. Accordingly, Wnt inhibitors suitable for use in the invention include:
(1) inhibitors of Wnt secretion (e.g. inhibitors of Porc, such as LGK974, IWP-1 or IWP-2),
(2) competitive and non-competitive inhibitors of the interaction between Wnt or Rspondin and their respective receptors (e.g. OMP-18R5, OMP54F28),
(3) factors that promote the degradation of components of the Wnt receptor complex, such as LRP (e.g. niclosamide) and factors that promote the degradation of Rspondin receptors, such as Znrf3 and/or Rnf43 or factors that activate Znrf3 and/or Rnf43,
(4) inhibitors of Dishevelled family proteins, such as inhibitors that reduce the binding of Dishevelled family proteins to Frizzled receptors and/or components of the destruction complex (e.g. Dapper family proteins, FJ9, sulindac, 3289-8625, J01-017a, NSC668036) or inhibitors that downregulate the expression of Dishevelled family proteins (e.g. niclosamide),
(5) factors that promote destruction complex activity, including (a) inhibitors of phosphatases (e.g. PP1, PP2A and/or PP2C) that dephosphorylate components of the destruction complex, such as axin and/or APC (e.g. okadaic acid or tautomycin) and (b) inhibitors of kinases (e.g. p38 MAPK, PKA, PKB, PKC, p90RSK or p70S6K) that phosphorylate GSK-3 (e.g. SB239063, SB203580 or Rp-8-Br-cAMP),
(6) inhibitors of the deoligomerisation of the destruction complex, such as inhibitors of Tankyrases 1 and/or 2 (e.g. XAV939, IWR1, JW74, JW55, 2-[4-(4-fluorophenyl)piperazin-1-yl]-6-methylpyrimidin-4(3H)-one or PJ34), and
(7) inhibitors of β-catenin target gene expression, including inhibitors of the β-catenin:TCF/Lef transcription complex, such as inhibitors that disrupt the β-catenin:TCF-4 complex (e.g. iCRT3, CGP049090, PKF118310, PKF115-584, ZTM000990, PNU-74654, BC21, iCRT5, iCRT14 or FH535) and inhibitors of the histone deacetylase SIRT1 (e.g. cambinol).

In some embodiments, the differentiation medium of the invention comprises a Wnt inhibitor. Any suitable Wnt inhibitor may be used as described in (1)-(7) above. For instance, in one preferred embodiment, the Wnt inhibitor is an inhibitor of Wnt secretion, such as a Porc inhibitor, e.g. selected from IWP-2, IWP-1 and LGK974. In another embodiment, the Wnt inhibitor is an inhibitor of β-catenin target gene expression, for example, an inhibitor of the β-catenin:TCF/Lef transcription complex or an inhibitor of the histone deacetylase SIRT1 (e.g. cambinol). In some embodiments, the inhibitor of the β-catenin:TCF/Lef transcription complex is an inhibitor that disrupts the β-catenin:TCF-4 complex, for example an inhibitor selected from iCRT3, CGP049090, PKF118310, PKF115-584, ZTM000990, PNU-74654, BC21, iCRT5, iCRT14 and FH535.

In some embodiments, the Wnt inhibitor is selected from IWP-2, OMP-18R5, OMP54F28, LGK974, 3289-8625, FJ9, NSC 668036, IWR1 and XAV939.

In some embodiments, the Wnt inhibitor is selected from iCRT3, PFK115-584, CGP049090, iCRT5, iCRT14 and FH535.

In some embodiments, the Wnt inhibitor is one of the compounds listed in Table 1 below.

TABLE 1
Wnt inhibitors

| Structure | Compound | Target |
|---|---|---|
| 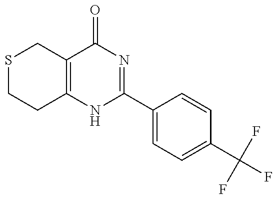 [SMILES: O=c1nc(-c2ccc(C(F)(F)F)cc2)[nH]c2c1CSCC2] | XAV939 | Tankyrases1, 2 |
| 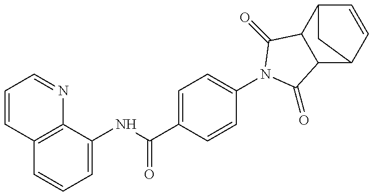 [SMILES: O=C(Nc1cccc2cccnc12)c1ccc(N2C(=O)C3C4C=CC(C4)C3C2=O)cc1] | IWR1 | Tankyrases1, 2 |
| 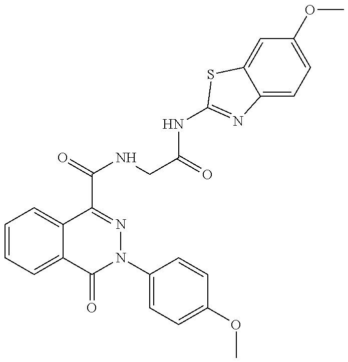 [SMILES: COc1ccc(-n2nc(C(=O)NCC(=O)Nc3nc4ccc(OC)cc4s3)c3ccccc3c2=O)cc1] | IWP-1 | Porcupine |
| 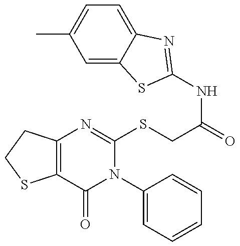 [SMILES: Cc1ccc2nc(NC(=O)CSc3nc4c(c(=O)n3-c3ccccc3)SCC4)sc2c1] | IWP-2 | Porcupine |
| 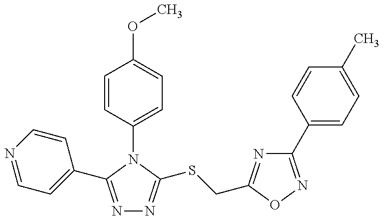 [SMILES: COc1ccc(-n2c(SCc3nc(-c4ccc(C)cc4)no3)nnc2-c2ccncc2)cc1] | JW74 | Tankyrases1, 2 |
| 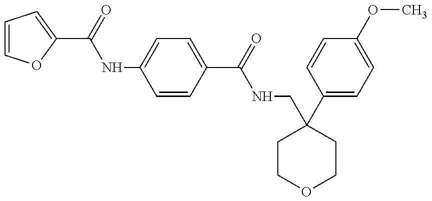 [SMILES: COc1ccc(C2(CNC(=O)c3ccc(NC(=O)c4ccco4)cc3)CCOCC2)cc1] | JW55 | Tankyrases1, 2 |
| 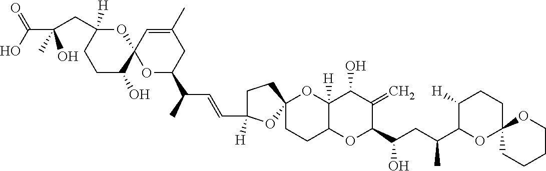 | Okadaic acid | PP2A phosphatase |
| 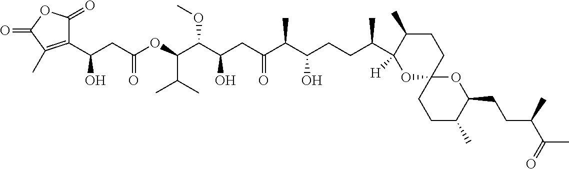 [SMILES: CO[C@H]([C@H](O)CC(=O)[C@@H](C)[C@@H](O)CC[C@@H](C)[C@@H]1O[C@]2(CC[C@@H](C)[C@H](CC[C@@H](C)C(C)=O)O2)CC[C@@H]1C)[C@H](OC(=O)C[C@@H](O)C1=C(C)C(=O)OC1=O)C(C)C] | Tautomycin | PP1 phosphatase |
| 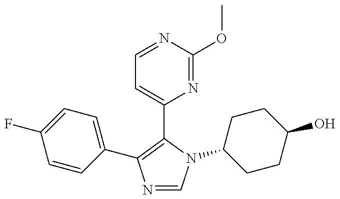 [SMILES: COc1nccc(-c2c(-c3ccc(F)cc3)ncn2[C@H]2CC[C@H](O)CC2)n1] | SB239063 | p38 MAPK |
| 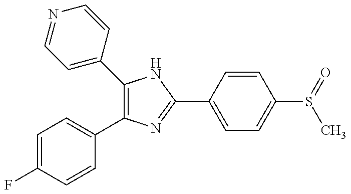 [SMILES: CS(=O)c1ccc(-c2nc(-c3ccc(F)cc3)c(-c3ccncc3)[nH]2)cc1] | SB203580 | p38 MAPK |
| 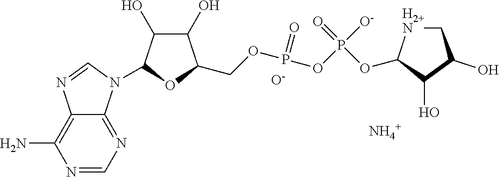 | ADP-HPD | PARG |
| 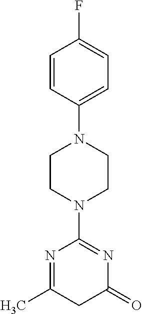 [SMILES: CC1=NC(N2CCN(c3ccc(F)cc3)CC2)=NC(=O)C1] | 2-[4-(4- fluorophenyl) piperazin- 1-yl]-6- methylpyrimidin- 4(3H)-one | Tankyrases1, 2 |
| 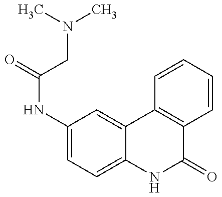 [SMILES: CN(C)CC(=O)Nc1ccc2[nH]c(=O)c3ccccc3c2c1] | PJ34 | Tankyrases1, 2 |
| 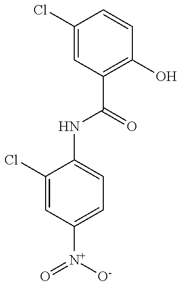 [SMILES: O=C(Nc1ccc([N+](=O)[O-])cc1Cl)c1cc(Cl)ccc1O] | Niclosamide | Downregulates Dvl-2, triggers LRP6 degradation |
| 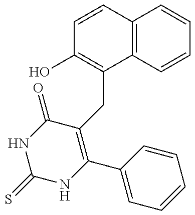 [SMILES: O=c1[nH]c(=S)[nH]c(-c2ccccc2)c1Cc1c(O)ccc2ccccc12] | Cambinol | SIRT1 |
| 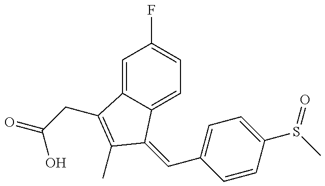 [SMILES: CC1=C(CC(=O)O)c2cc(F)ccc2/C1=C\c1ccc(S(C)=O)cc1] | Sulindac | PDZ domain of Dishevelled |
| 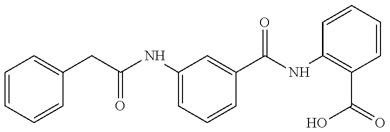 [SMILES: O=C(Cc1ccccc1)Nc1cccc(C(=O)Nc2ccccc2C(=O)O)c1] | 3289-8625 | Dishevelled |
| 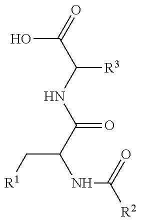 | Scaffold A for series of analogs | Dishevelled |
| 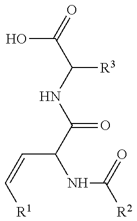 | Scaffold B for series of analogs | Dishevelled |
| 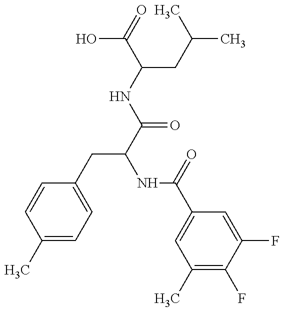 [SMILES: Cc1ccc(CC(NC(=O)c2cc(C)c(F)c(F)c2)C(=O)NC(CC(C)C)C(=O)O)cc1] | J01-017a | Dishevelled |
| 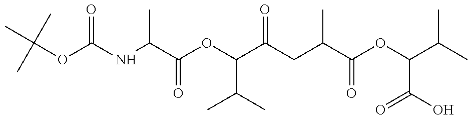 [SMILES: CC(CC(=O)C(OC(=O)C(C)NC(=O)OC(C)(C)C)C(C)C)C(=O)OC(C(=O)O)C(C)C] | NSC668036 | Dishevelled |
| 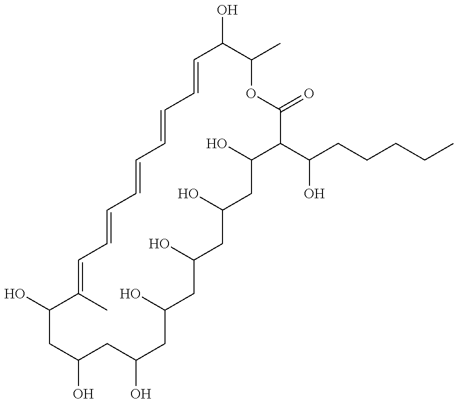 [SMILES: CCCCCC(O)C1C(=O)OC(C)C(O)/C=C/C=C/C=C/C=C/C=C(\C)C(O)CC(O)CC(O)CC(O)CC(O)CC(O)CC1O] | Filipin | Caveolin- mediated endocytosis |
| 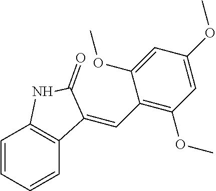 [SMILES: COc1cc(OC)c(/C=C2\C(=O)Nc3ccccc32)c(OC)c1] | IC261 | CK1ε/δ |
| 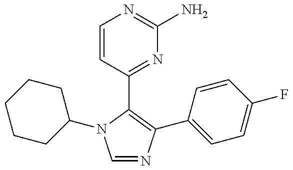 [SMILES: Nc1nccc(-c2c(-c3ccc(F)cc3)ncn2C2CCCCC2)n1] | PF670462 | CK1δ and CK1ε |
| 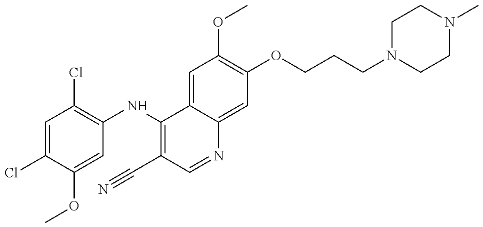 [SMILES: COc1cc(Nc2c(C#N)cnc3cc(OCCCN4CCN(C)CC4)c(OC)cc23)c(Cl)cc1Cl] | Bosutinib | Src kinase |
| 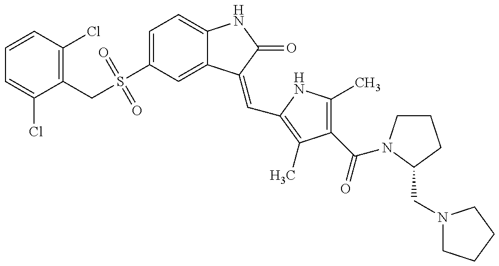 [SMILES: Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1] | PHA665752 | c-Met |
| 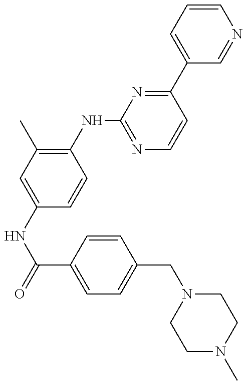 [SMILES: Cc1cc(NC(=O)c2ccc(CN3CCN(C)CC3)cc2)ccc1Nc1nccc(-c2cccnc2)n1] | Imatinib | Different tyrosine kinases |
| 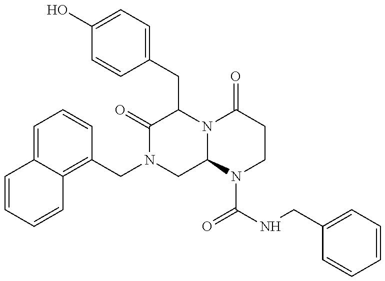 [SMILES: O=C1C(Cc2ccc(O)cc2)N2C(=O)CCN(C(=O)NCc3ccccc3)[C@H]2CN1Cc1cccc2ccccc12] | ICG-001 | CREB binding protein (CBP) |
| 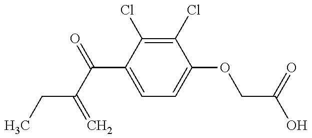 [SMILES: C=C(CC)C(=O)c1ccc(OCC(=O)O)c(Cl)c1Cl] | Ethacrynic acid | Lef-1 |
| 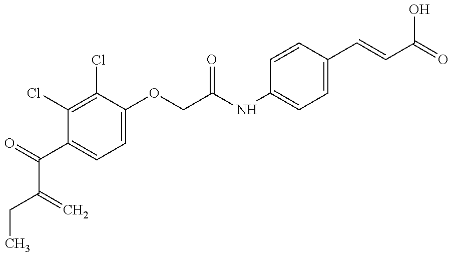 [SMILES: C=C(CC)C(=O)c1ccc(OCC(=O)Nc2ccc(/C=C/C(=O)O)cc2)c(Cl)c1Cl] | Ethacrynic acid derivative | Lef-1 |
| 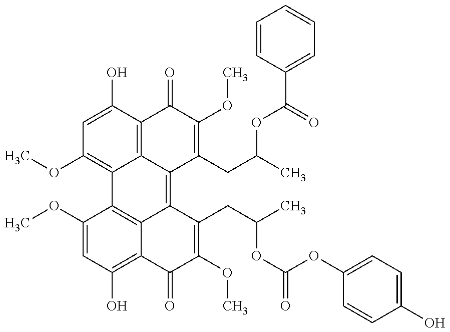 [SMILES: COc1c(CC(C)OC(=O)Oc2ccc(O)cc2)c2c3c(CC(C)OC(=O)c4ccccc4)c(OC)c(=O)c4c(O)cc(OC)c(c5c(OC)cc(O)c(c1=O)c52)c43] | PKF115-584 | β-catenin |
| 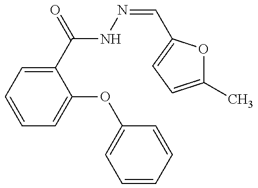 [SMILES: Cc1ccc(/C=N\NC(=O)c2ccccc2Oc2ccccc2)o1] | PNU-74654 | β-catenin |
| 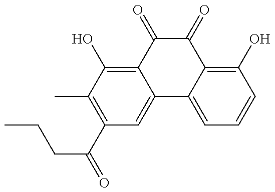 [SMILES: CCCC(=O)c1cc2c(c(O)c1C)C(=O)C(=O)c1c(O)cccc1-2] | PKF118-744 | β-catenin |
| 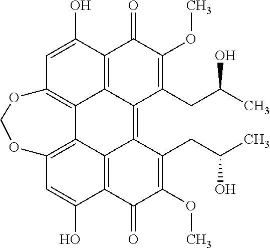 [SMILES: COc1c(C[C@H](C)O)c2c3c(C[C@H](C)O)c(OC)c(=O)c4c(O)cc5c(c6c(cc(O)c(c1=O)c62)OCO5)c43] | CGP049090 | β-catenin |
| 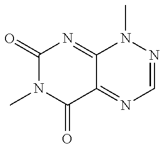 | PKF118-310 | β-catenin |
| 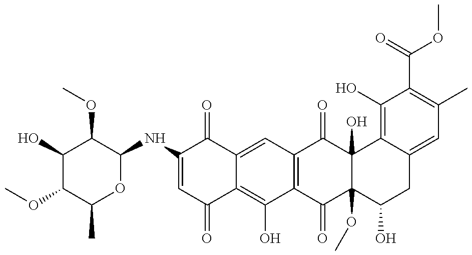 | ZTM000990 | β-catenin |
| 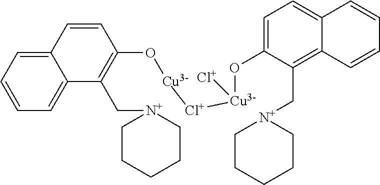 | BC21 | β-catenin |
| 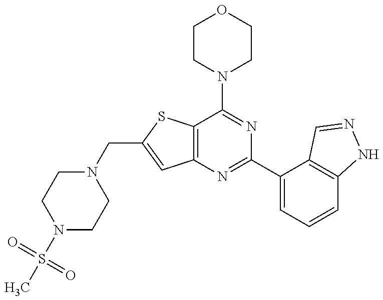 [SMILES: CS(=O)(=O)N1CCN(Cc2cc3nc(-c4cccc5[nH]ncc45)nc(N4CCOCC4)c3s2)CC1] | GDC-0941 | PI3K |
| 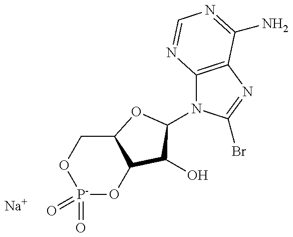 | Rp-8-Br-cAMP | PKA |

Other Wnt inhibitors suitable for use with the invention include: TMEM88, KY-02061, KY-02327, BMD4702, DK-520, pyrvinium, derricin, derricidin, carnosic acid, windorphen, IWP-L6, Wnt-C59, ETC-159, E7449 and WIKI4.

In some embodiments, a differentiation medium of the invention comprises one or more of TMEM88, KY-02061, KY-02327, BMD4702, DK-520, pyrvinium, derricin, derricidin, carnosic acid, windorphen, IWP-L6, Wnt-C59, ETC-159, E7449, WIKI4 or any of the Wnt inhibitors listed in table 1.

The Wnt inhibitor is preferably added to the media in an amount effective to inhibit a Wnt activity in a cell by at least 10%, more preferred at least 20%, more preferred at least 30%, more preferred at least 50%, more preferred at least 70%, more preferred at least 90%, more preferred 100%, relative to a level of said Wnt activity in the absence of said molecule, as assessed in the same cell type. As is known to a skilled person, Wnt activity can be determined by measuring the transcriptional activity of Wnt, for example by pTOPFLASH and pFOPFLASH Tcf luciferase reporter constructs (Korinek et al. (1997) Science 275:1784-1787). Wnt activity may also be determined using LEADING LIGHT® Wnt Reporter Assay Starter Kit (Enzo Life Sciences, cat. no. ENZ-61001-0001), which uses an engineered 3T3 mouse fibroblast cell line expressing the firefly luciferase reporter gene under the control of Wnt-responsive promoters (TCF/LEF). Additionally, Grimaldi et al. (Frontiers in Pharmacology 9: 1160) describes a cell model suitable for high-throughput screening of Wnt inhibitors, which comprises DLD-1 cells stably transfected with a luciferase TCF reporter plasmid. Furthermore, a cell-based HTRF (Homogeneous Time Resolved Fluorescence) assay for Wnt signalling activity, which detects protein level and phosphorylation of GSK3 and β-catenin, is described in Romier et al. (“New cell-based HTRF® assays for the exploration of Wnt signalling pathway” Cisbio Bioassays). New Wnt inhibitors can therefore easily be identified by a skilled person using assays known in the art.

In some embodiments, the differentiation medium of the invention comprises a Wnt inhibitor at a concentration of 0.01-150 μM, 0.1-150 μM, 0.5-100 μM, 0.1-100 μM, 0.5-50 μM, 1-100 μM or 10-80 μM, 1-20 μM or 1-5 μM.

In some embodiments, the differentiation medium of the invention comprises IWP-2 at a concentration of 0.01-150 μM, 0.1-100 μM, 0.5-50 μM, 1-20 μM or 1-5 μM. For example, in some embodiments, the differentiation medium of the invention comprises IWP-2 at a concentration of about 1.5 μM.

In some embodiments, the differentiation medium does not comprise a Wnt agonist that binds and activates the Wnt receptor complex including any and all of the Wnt family proteins and Rspondin.

In other embodiments, the differentiation medium further comprises a Wnt agonist, such as R-spondin 1-4 or a biologically active fragment or variant thereof. As described above R-spondins enhance Wnt signalling at receptors at the cell surface. It is hypothesised that some Wnt signalling may be required to direct the cells towards the secretory (rather than absorptive) lineage. Therefore, in some embodiments, the differentiation medium comprises both a Wnt agonist (particularly an R-spondin) and a Wnt inhibitor. For example, in some embodiments, the differentiation medium comprises an R-spondin and a Porc inhibitor, such as IWP-2. In some embodiments, the R-spondin is used at a final concentration of between 1 and 1000 ng/ml, between 50 and 1000 ng/ml or between 100 and 1000 ng/ml. In preferred embodiments, the R-spondin is used at a final concentration of about 250 ng/ml.

### Receptor Tyrosine Kinase Ligands

In some embodiments, an expansion or differentiation medium of the invention further comprises a receptor tyrosine kinase ligand.

Receptor tyrosine kinases (RTKs) are high-affinity cell surface receptors for polypeptide growth factors, cytokines, and hormones. RTKs and their ligands are described in detail in Trenker and Jura (Current Opinion in Cell Biology 2020, 63:174-185). RTKs are key regulators of cell maintenance, growth and development, and also to have a critical role in the development and progression of many types of cancer. RTK activity may be determined using the Proteome Profiler Human Phospho-RTK Array Kit (R&D systems), which determines the relative phosphorylation of 49 human RTKs.

In the context of the invention, a receptor tyrosine kinase ligand is any ligand that activates an RTK. Many receptor tyrosine kinase ligands are mitogenic growth factors. Thus in some embodiments, the one or more receptor tyrosine kinase ligands in the differentiation medium comprises one or more mitogenic growth factor.

There are approximately 20 different known classes of RTKs, including RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family), RTK class XVII (MuSK receptor family). In some embodiments, the one or more receptor tyrosine kinase ligands comprises ligands for one or more, or all of these 20 classes of RTKs.

RTK class I, for example, includes EGFR/ErbB1, ErbB2/HER2/neu, ErbB3/HER3 and ErbB4/HER4. Ligands of the RTK class I family include EGF (an ErbB1 ligand) and neuregulins (ErbB3/4 ligands), which have been shown to be useful in organoid culture (e.g. see WO/2016/083613). Ligands from RTK class IV (FGF receptor family) and RTK class VI (HGF receptor family) and ligands from RTK class II (Insulin receptor family) have also been shown to be useful in organoid culture. Therefore, in some embodiments, the one or more receptor tyrosine kinase ligand comprises ligands for one or more of RTK class I, RTK class II, RTK class IV or RTK class VI.

In some embodiments, the receptor tyrosine kinase ligand in the expansion or differentiation medium is selected from the group consisting of epidermal growth factor (EGF), neuregulin, fibroblast growth factor (FGF), hepatocyte growth factor (HGF), and insulin-like growth factor (IGF). In some embodiments, the receptor tyrosine kinase ligand in the expansion or differentiation medium is selected from the group consisting of epidermal growth factor (EGF), neuregulin, fibroblast growth factor (FGF) and hepatocyte growth factor (HGF). Preferably, the receptor tyrosine kinase ligand is EGF. Any suitable concentration of a receptor tyrosine kinase ligand may be used, for example, a concentration of about 50 ng/mL EGF may be used.

### BMP Inhibitors

In some embodiments, the differentiation medium of the invention further comprises a BMP inhibitor.

BMPs are small signalling molecules that bind to two classes of cell surface bone morphogenetic protein receptors (BMPR-I and BMPRII). The BMPR-I receptor class consists of three receptor types, activin receptor-like kinase-2 (ALK-2 or ActR-IA), ALK-3 (BMPR-IA) and ALK-6 (BMPR-IB). The BMPR-II receptor class is comprised of three receptor types, BMPR-II, ActR-IIA and ActR-IIB. Binding of BMPs results in the formation of heterotetrameric complexes containing two type I and two type II receptors. In addition to an extracellular binding domain, each BMP receptor contains an intracellular serine/threonine kinase domain. Following binding of BMPs, constitutively active type II receptor kinases phosphorylate type I receptor kinase domains that in turn phosphorylate BMP-responsive SMADs 1, 5, and 8, which can enter the cell nucleus and function as transcription factors. Phosphorylation of these specific SMADs results in various cellular effects, including growth regulation and differentiation. A BMP inhibitor is any inhibitor that results in a significant reduction in signaling via these pathways. For example, a BMP inhibitor may be able to disrupt the interaction of a BMP with a BMP receptor; bind to a BMP receptor and inhibit activation of downstream signalling; inhibit phosphorylation of Smad 1, Smad 5 or Smad 8; inhibit translocation of Smad 1, Smad 5 or Smad 8 to the nucleus; inhibit SMAD 1, SMAD 5 or SMAD 8 mediated transcription of target genes; or inhibit expression, folding or secretion of a BMP. In some embodiments, the BMP inhibitor reduces signaling via the BMPR-I receptor class. In some embodiments, the BMP inhibitor reduces signaling via BMPR-II receptor class. In some embodiments, the BMP inhibitor reduces signaling via SMAD 1/5/8. The inhibition may be direct or indirect.

Many BMP inhibitors are known in the art, e.g. as disclosed in Cuny, et al., (2008) Structure-activity relationship study of bone morphogenetic protein (BMP) signaling inhibitors. Bioorg Med Chem Lett 18: 4388-4392 and Sachez-Duffhues (2020) Bone 138:115472. Any of these BMP inhibitors are suitable for use in the methods of the invention. Methods for identifying suitable BMP inhibitors are known in the art. A suitable assay is described in Zilberberg et al., BMC Cell Biology 2007 8:41. Another suitable assay for a BMP inhibitor (in particular a BMP inhibitor that inhibits phosphorylation of Smad 1, 5 or 8 via ALK2 and ALK3) can be identified by a person skilled in the art using the cytobot cellular ELISA assay described in Cuny, et al., (2008) Structure-activity relationship study of bone morphogenetic protein (BMP) signaling inhibitors. Bioorg Med Chem Lett 18: 4388-4392. Further assays for BMP inhibitors are described in Dinter et al. (2019) Methods Mol Biol 1891:221-233.

In some embodiments the BMP inhibitor is selected from noggin, chordin, follistatin, gremlin, tsg (twisted gastrulation), sog (short gastrulation), dorsomorphin and LDN193189. In some embodiments, the BMP inhibitor is selected from:
a. dorsomorphin or LDN193189 or an analog or variant thereof, and/or
b. noggin, sclerostin, chordin, CTGF, follistatin, gremlin, tsg, sog or an analog or variant thereof.

In preferred embodiments, the BMP inhibitor is noggin. Noggin is particularly suitable for in vitro culture methods. Preferably the noggin is recombinant noggin.

In some embodiments, noggin is included in the expansion or differentiation medium at a final concentration of between 1 and 1000 ng/ml, between 10 and 1000 ng/ml, between 100 and 1000 ng/ml, between 1 and 500 ng/ml, between 1 and 200 ng/ml, between 1 and 100 ng/ml, between 10 and 500 ng/ml, between 20 and 500 ng/ml, between 10 and 200 ng/ml, between 20 and 200 ng/ml, between 50 and 500 ng/ml, or between 50 and 200 ng/ml. In preferred embodiments, noggin is included in the expansion or differentiation medium at a final concentration of about 100 ng/ml.

In some embodiments, noggin is provided in the form of noggin-conditioned medium, for example, Noggin-Fc Fusion Protein conditioned medium (U-Protein Express, cat no. N002). In some embodiments, the expansion or differentiation medium comprises noggin-conditioned medium at a final concentration of between 0.1-10% or between 0.5-5%. In preferred embodiments, the expansion or differentiation medium comprises noggin-conditioned medium at a final concentration of about 1-2%.

### BMP Pathway Activators

In some embodiments, the differentiation medium comprises a BMP pathway activator. In some embodiments, the differentiation medium does not comprise a BMP inhibitor (e.g. Noggin). In some embodiments, the differentiation medium comprises a BMP pathway activator and does not comprise a BMP inhibitor (e.g. Noggin).

Methods for identifying suitable BMP pathway activators are known in the art. A suitable assay for measuring BMP activity is described in Zilberberg et al., BMC Cell Biology 2007 8:41.

In some embodiments, the BMP pathway activator is selected from BMP7, BMP4 and BMP2. BMP4 is preferred.

In some embodiments, the BMP pathway activator, such as BMP4 is present in the differentiation medium at at least 0.01 ng·ml, at least 0.1 ng/ml, at least 1 ng/ml, at least 10 ng/ml, at least 20 ng/ml, at least 25 ng/ml, at least 100 ng/ml, at least 500 ng/ml, at least 1 μg/ml, at least 10 μg/ml or at least 50 μg/ml. In some embodiments, the BMP pathway activator, such as BMP4 is present in the differentiation medium from about 0.01 ng/ml to about 500 ng/ml, from about 1 ng/ml to about 500 ng/ml, from about 10 ng/ml to about 500 ng/ml, from about 20 ng/ml to about 500 ng/ml. In some embodiments, the BMP pathway activator, such as BMP4, is present in the differentiation medium from about 0.01 ng/ml to about 200 ng/ml, from about 0.1 ng/ml to about 100 ng/ml, from about 1 ng/ml to about 100 ng/ml. In some embodiments, the BMP pathway activator, such as BMP4 is present in the differentiation medium at about 10 ng/ml.

In some embodiments, the differentiation medium does not comprise a BMP pathway activator.

### Notch Inhibitors

In some embodiments, the differentiation medium comprises a Notch inhibitor. Any suitable Notch inhibitor may be used.

Notch is a transmembrane surface receptor that can be activated through multiple proteolytic cleavages, one of them being cleavage by a complex of proteins with protease activity, termed gamma-secretase. Gamma-secretase is a protease that performs its cleavage activity within the membrane. Gamma-secretase is a multicomponent enzyme and is composed of at least four different proteins, namely, presenilins (presenilin 1 or 2), nicastrin, PEN-2 and APH-I. Presenilin is the catalytic centre of gamma-secretase. On ligand binding the Notch receptor undergoes a conformational change that allows ectodomain shedding through the action of an ADAM protease which is a metalloprotease. This is followed immediately by the action of the gamma-secretase complex which results in the release of the Notch intracellular domain (NICD). NICD translocates to the nucleus where it interacts with CSL (C-promoter-binding factor/recombinant signal-sequence binding protein Jx/Supressor-of-Hairless/Lagl). The binding of NICD converts CSL from a transcriptional repressor to an activator which results in the expression of Notch target genes.

In some embodiments, the Notch inhibitor is an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases.

In some embodiments the Notch inhibitor is a gamma-secretase inhibitor, for example DAPT, dibenzazepine (DBZ), benzodiazepine (BZ) or LY-411575. One or more Notch inhibitors may be used, for example, 2, 3, 4 or more.

In some embodiments, the Notch inhibitor (e.g. DAPT) is used at a concentration of 0.001-200 mM, 0.01-100 mM, 0.1-50 mM, 0.1-20 mM, 0.5-10 mM or 0.5-5 mM. In some embodiments, the differentiation medium comprises DAPT at a concentration of about 10 μM.

Notch inhibitors are commercially available from e.g. MedChemExpress. Further Notch inhibitors may be identified by assaying Notch signalling activity, for example, using the Notch1 Pathway Reporter Kit (BPS Bioscience) or the TaqMan™ Array Human Notch Signaling plate (Applied Biosystems).

### EGFR Pathway Inhibitors

In some embodiments, the differentiation medium of the invention comprises an EGFR pathway inhibitor. Any suitable inhibitor as defined herein may be used.

Epidermal growth factor receptor (EGFR), also known as ErbB1 or HER1, is a cell surface receptor for members of the epidermal growth factor (EGF) family of extracellular protein ligands. EGFR belongs to the HER family of receptors which comprise four related proteins (EGFR(HER1/ErbB1), ErbB2(HER2), ErbB3(HER3) and ErbB4(HER4)). The HER receptors are known to be activated by binding to different ligands, including EGF, TGFA, heparin-binding EGF-like growth factor, amphiregulin, betacellulin, and epiregulin. After a ligand binds to the extracellular domain of the receptor, the receptor forms functionally active dimers (EGFR-EGFR (homodimer) or EGFR-HER2, EGFR-HER3, EGFR-HER4 (heterodimer)). Dimerization induces the activation of the tyrosine kinase domain, which leads to autophosphorylation of the receptor on multiple tyrosine residues. This leads to recruitment of a range of adaptor proteins (such as SHC, GRB2) and activates a series of intracellular signalling cascades to affect gene transcription.

The pathways mediating downstream effects of EGFR have been well studied and three major signalling pathways have been identified. The first pathway involves RAS-RAF-MAPK pathway, where phosphorylated EGFR recruits the guanine-nucleotide exchange factor via the GRB2 and Shc adapter proteins, activating RAS and subsequently stimulating RAF and the MAP kinase pathway to affect cell proliferation, tumor invasion, and metastasis. Activated RAS activates the protein kinase activity of RAF kinase. RAF kinase phosphorylates and activates MEK (also known as MAP2K or MAPKK), which phosphorylates and activates a MAP kinase (also known as an ERK, an extracellular signal-regulated kinase). The second pathway involves PI3K/AKT pathway, which activates the major cellular survival and anti-apoptosis signals via activating nuclear transcription factors such as NFKB. The third pathway involves JAK/STAT pathway which is also implicated in activating transcription of genes associated with cell survival. EGFR activation may also lead to phosphorylation of PLCG and subsequent hydrolysis of phosphatidylinositol 4,5 biphosphate (PIP2) into inositol 1,4,5-triphosphate (IP3) and diacylglycerol (DAG), resulting in activation of protein kinase C (PRKC) and CAMK.

EGFR inhibitors, such as anti-EGFR monoclonal antibodies and small-molecule EGFR tyrosine kinase inhibitors, are available. Some anti-EGFR antibodies, such as cetuximab and panitumumab, bind to the extracellular domain of EGFR monomer and compete for receptor binding by the endogenous ligands; in this way they block ligand-induced receptor activation. Some small molecule EGFR inhibitors, such as erlotinib, gefitinib and lapatinib, compete with ATP to bind the kinase domain of EGFR which in turn inhibits EGFR autophosphorylation and downstream signalling.

The EGFR signalling pathway and a number of EGFR inhibitors are described in Singh et al. (2016) Mini-Reviews in Medicinal Chemistry 16:1134-1166. Further EGFR inhibitors may be identified by assaying EGFR signalling activity, for example, using the EGFR Kinase Assay Kit (BPS Bioscience).

One or more EGFR pathway inhibitors may be used, for example, 2, 3, 4 or more.

The EGFR pathway inhibitor is preferably added to the media in an amount effective to inhibit an EGFR pathway activity in a cell by at least 10%, more preferred at least 20%, more preferred at least 30%, more preferred at least 50%, more preferred at least 70%, more preferred at least 90%, more preferred 100%, relative to a level of said EGFR pathway activity in the absence of said molecule, as assessed in the same cell type. As is known to a skilled person, EGFR pathway activity can be measured in a variety of ways. For example, an assay for monitoring EGFR activity and inhibitor sensitivities is described in Ghosh et al. (2013) Assay and Drug Development Technologies 11(1):44-51. This particular assay involves peptide substrates that are covalently immobilized to magnetic beads. After kinase reactions, the beads are washed and phosphorylation of the peptides is detected by chemifluorescence using an HRP-conjugated primary antibody against phosphorylated tyrosine. The fluorescence intensity measured is directly proportional to substrate phosphorylation, which in turn is proportional to EGFR kinase activity. This assay could also be used to screen for inhibitors of other kinases in the EGFR pathway (e.g. RAS, RAF, MEK or ERK). An alternative method for assaying kinase activity involves detecting incorporation of terminal phosphate from P³²-labelled ATP. New EGFR pathway inhibitors can therefore easily be identified by a skilled person using an assay known in the art.

In some embodiments, the EGFR pathway inhibitor is an EGFR inhibitor that inhibits EGFR kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is a RAS inhibitor that inhibits RAS kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an RAF inhibitor that inhibits RAF kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an MEK inhibitor that inhibits MEK kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an ERK inhibitor that inhibits ERK kinase activity by at least 10%, more preferred at least 20%, more preferred at least 30%, more preferred at least 50%, more preferred at least 70%, more preferred at least 90%, more preferred 100%.

In some embodiments, EGF is present in the differentiation medium at a concentration of less than 1 mM.

In some embodiments, the EGFR pathway inhibitor is an EGFR inhibitor, such as Gefitinib (Santa Cruz Biotechnology), AG-18, AG-490 (tyrphostin B42), AG-1478 (tyrphostin AG-1478), AZ5104, AZD3759, brigatinib, erlotinib, cetuximab, CL-387785 (EKI-785), CNX-2006, icotinib, necitumumab, osimertinib (AZD9291), OSI-420, PD153035 HCl, PD168393, pelitinib (EKB-569), rociletinib (CO-1686, AVL-301), TAK-285, tyrphostin 9, vandetanib, WHI-P154, WZ3146, WZ4002, WZ8040, panitumumab, zalutumumab, nimotuzumab or matuzumab. In some embodiments, the EGFR inhibitor binds to the extracellular domain of EGFR monomer and competes for receptor binding by EGF. In some embodiments, the EGFR inhibitor competes with ATP to bind the kinase domain of EGFR. One or more EGFR inhibitors may be used, for example, 2, 3, 4 or more.

In some embodiments, the EGFR pathway inhibitor is an EGFR and ErbB-2 inhibitor, such as Afatinib (Selleckchem), afatinib dimaleate, AC480 (BMS-599626), AEE788 (NVP-AEE788), AST-1306, canertinib, CUDC-101, dacomitinib, lapatinib, neratinib, poziotinib (HM781-36B), sapitinib (AZD8931) or varlitinib. One or more EGFR and ErbB-2 inhibitors may be used, for example, 2, 3, 4 or more.

In some embodiments, the EGFR pathway inhibitor is an inhibitor of the RAS-RAF-MAPK pathway. In some embodiments, the EGFR pathway inhibitor is an inhibitor of the PI3K/AKT pathway. In some embodiments, the EGFR pathway inhibitor is an inhibitor of the JAK/STAT pathway.

In some embodiments, the EGFR pathway inhibitor is a RAF inhibitor, such as GW5074, ZM 336372, NVP-BHG712, TAK-632, darafenib (GSK2118436), sorafenib, sorafenib tosylate, PLX-4720, AZ 628, CEP-32496 or vemurafenib (PLX4032, RG7204).

In some embodiments, the EGFR pathway inhibitor is an MEK inhibitor, such as PD0325901 (Sigma Aldrich). In some embodiments, the EGFR pathway inhibitor is an ERK inhibitor, such as SCH772984 (Selleckchem).

In some embodiments, the EGFR pathway inhibitor is used at a concentration of 0.01-200 μM, 0.01-100 μM, 0.1-50 μM, or 0.1-20 μM. For example, in some embodiments, the differentiation medium comprises PD0325901 at a concentration of about 100 nM.

### Basal Medium

The expansion and differentiation media of the invention comprise a basal medium. The basal medium is any suitable basal medium for animal or human cells, subject to the limitations provided herein.

Basal media for animal or human cell culture typically contain a large number of ingredients, which are necessary to support maintenance of the cultured cells. Suitable combinations of ingredients can readily be formulated by the skilled person, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature and above. In some embodiments, the culture medium is further supplemented with one or more standard cell culture ingredient, for example selected from amino acids, vitamins, lipid supplements, inorganic salts, a carbon energy source, and a buffer.

The skilled person will understand from common general knowledge the types of culture media that might be used as the basal medium in the expansion or differentiation medium of the invention. Potentially suitable cell culture media are available commercially, and include, but are not limited to, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

For example, the basal medium may be selected from DMEM/F12 and RPMI 1640 supplemented with glutamine, insulin, penicillin/streptomycin and transferrin. In a further preferred embodiment, Advanced DMEM/F12 or Advanced RPMI is used, which is optimized for serum free culture and already includes insulin. In this case, said Advanced DMEM/F12 or Advanced RPMI medium is preferably supplemented with glutamine and penicillin/streptomycin. AdDMEM/F12 (Invitrogen) supplemented with N2 and B27 is also preferred. Preferably, the basal medium is Advanced DMEM/F12. More preferably, the basal medium comprises Advanced DMEM/F12, glutamine and B27.

In preferred embodiments, the basal medium comprises Advanced DMEM/F12, HEPES, penicillin/streptomycin, Glutamine, N-Acetylcysteine and B27.

In more preferred embodiments, the basal medium comprises or consists of Advanced DMEM/F12 supplemented with penicillin/streptomycin, 10 mM HEPES, Glutamax, B27 (all from Life Technologies, Carlsbad, CA) and about 1.25 mM N-acetylcysteine (Sigma).

It is furthermore preferred that said basal culture medium is supplemented with a purified, natural, semi-synthetic and/or synthetic growth factor and does not comprise an undefined component such as fetal bovine serum or fetal calf serum. Various different serum replacement formulations are commercially available and are known to the skilled person. Where a serum replacement is used, it may be used at between about 1% and about 30% by volume of the medium, according to conventional techniques.

The expansion and differentiation media used in the invention may comprise serum, or may be serum-free and/or serum-replacement free, as described elsewhere herein. Culture media and cell preparations are preferably GMP processes in line with standards required by the FDA for biologics products and to ensure product consistency.

In preferred embodiments, the expansion and differentiation media are feeder cell-free and/or do not comprise feeder cell-conditioned medium.

In preferred embodiments, the expansion and differentiation media do not contain undefined components.

In preferred embodiments, when culturing human cells, human growth factors are used to avoid any xeno-contamination—human cultures which do not contain any non-human animal components, for example, are also known as xeno-free. In preferred embodiments the expansion and/or differentiation media are xeno-free.

The expansion and differentiation media of the invention will normally be formulated in deionized, distilled water. The expansion and differentiation media of the invention will typically be sterilized prior to use to prevent contamination, e.g. by ultraviolet light, heating, irradiation or filtration. The expansion and differentiation media may be frozen (e.g. at −20° C. or −80° C.) for storage or transport. The medium may contain one or more antibiotics to prevent contamination. The medium may have an endotoxin content of less that 0.1 endotoxin units per ml, or may have an endotoxin content less than 0.05 endotoxin units per ml. Methods for determining the endotoxin content of culture media are known in the art.

A preferred basal culture medium is a defined synthetic medium that is buffered at a pH of 7.4 (preferably with a pH 7.2-7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

### Additional Factors

### p38 MAPK Inhibitor

In some embodiments of the invention, the expansion medium further comprises a p38 MAPK inhibitor, also referred to herein as a p38 inhibitor, meaning any inhibitor that, directly or indirectly, negatively regulates p38 signalling. In some embodiments, an inhibitor according to the invention binds to and reduces the activity of p38 (GI number 1432). p38 protein kinases are part of the family of mitogen-activated protein kinases (MAPKs). MAPKs are serine/threonine-specific protein kinases that respond to extracellular stimuli, such as environmental stress and inflammatory cytokines, and regulate various cellular activities, such as gene expression, mitosis, differentiation, proliferation, and cell survival/apoptosis. The p38 MAPKs exist as α, β, β2, γ and δ isoforms. A p38 inhibitor is an agent that binds to and reduces the activity of at least one p38 isoform. Various methods for determining if a substance is a p38 inhibitor are known, and might be used in conjunction with the invention. Examples include: phospho-specific antibody detection of phosphorylation at Thr180/Tyr182, which provides a well-established measure of cellular p38 activation or inhibition; biochemical recombinant kinase assays; tumor necrosis factor alpha (TNFα) secretion assays; and DiscoverRx high throughput screening platform for p38 inhbitors (see http://www.discoverx.com/kinases/literature/biochemical/collaterals/DRx_poster_p388% 20KBA.pdf). Several p38 activity assay kits also exist (e.g. Millipore, Sigma-Aldrich).

Various p38 inhibitors are known in the art. In some embodiments, the inhibitor that directly or indirectly negatively regulates p38 signalling is selected from the group consisting of SB-202190, SB-203580, VX-702, VX-745, PD-169316, RO-4402257 and BIRB-796.

In one embodiment, the p38 inhibitor according to the invention binds to and reduces the activity of its target by more than 10%; more than 30%; more than 60%; more than 80%; more than 90%; more than 95%; or more than 99% compared to a control, as assessed by a cellular assay. Examples of cellular assays for measuring target inhibition are well known in the art as described above.

SB-203580 may be added to the expansion medium at a concentration of between 50 nM and 100 μM, or between 100 nM and 50 μM, or between 1 μM and 50 PM. For example, SB-203580 may be added to the expansion medium at approximately 10 μM.

### TGF-Beta Inhibitor

In some embodiments, the expansion or differentiation medium further comprises a TGF-beta inhibitor.

TGF-beta signalling is involved in many cellular functions, including cell growth, cell fate and apoptosis. Signalling typically begins with binding of a TGF-beta superfamily ligand to a type II receptor which recruits and phosphorylates a type I receptor. The type I receptor then phosphorylates SMADs, which act as transcription factors in the nucleus and regulate target gene expression.

The TGF-beta inhibitor signalling pathway has previously been implicated in promoting the differentiation of progenitor cells. For example, the addition of TGF-beta to liver explants facilitates the biliary differentiation in vitro (Clotman et al. (2005) Genes Dev. 19(16):1849-54). In addition, it has previously been shown that inclusion of a TGF-beta inhibitor in a differentiation medium can inhibit biliary duct cell-fate and trigger the differentiation of the cells towards a more hepatocytic phenotype (see WO 2012/168930). In particular, inclusion of a TGF-beta inhibitor (such as A83-01) in a differentiation medium was found to enhance the expression of mature hepatocyte markers and increase the number of hepatocyte-like cells.

The TGF-beta superfamily ligands comprise bone morphogenic proteins (BMPs), growth and differentiation factors (GDFs), anti-mullerian hormone (AMH), activin, nodal and TGF-betas. In general, Smad2 and Smad3 are phosphorylated by the ALK4, 5 and 7 receptors in the TGF-beta/activin pathway. By contrast, Smad1, Smad5 and Smad8 are phosphorylated as part of the bone morphogenetic protein (BMP) pathway. Although there is some cross-over between pathways, in the context of this invention, a “TGF-beta inhibitor” or an “inhibitor of TGF-beta signalling” is preferably an inhibitor of the TGF-beta pathway which acts via Smad2 and Smad3 and/or via ALK4, ALK5 or ALK7. Therefore, in some embodiments the TGF-beta inhibitor is not a BMP inhibitor, i.e. the TGF-beta inhibitor is not Noggin. In some embodiments, a BMP inhibitor is added to the culture medium in addition to the TGF-beta inhibitor. Thus the TGF-beta inhibitor may be any agent that reduces the activity of the TGF-beta signalling pathway, preferably the signalling pathway that acts via Smad2 and/or Smad3, more preferably the signalling pathway that acts via ALK4, ALK5 or ALK7.

There are many ways of disrupting the TGF-beta signalling pathway that are known in the art and that can be used in conjunction with this invention. For example, the TGF-beta signalling may be disrupted by: inhibition of TGF-beta expression by a small-interfering RNA strategy; inhibition of furin (a TGF-beta activating protease); inhibition of the pathway by physiological inhibitors; neutralisation of TGF-beta with a monoclonal antibody; inhibition with small-molecule inhibitors of TGF-beta receptor kinase 1 (also known as activin receptor-like kinase, ALK5), ALK4, ALK6, ALK7 or other TGF-beta-related receptor kinases; inhibition of Smad 2 and Smad 3 signalling e.g. by overexpression of their physiological inhibitor, Smad 7, or by using thioredoxin as an Smad anchor disabling Smad from activation (Fuchs, O. Inhibition of TGF-Signalling for the Treatment of Tumor Metastasis and Fibrotic Diseases. Current Signal Transduction Therapy, Volume 6, Number 1, January 2011, pp. 29-43(15)).

Various methods for determining if a substance is a TGF-beta inhibitor are known and might be used in conjunction with the invention. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al. (2005) Br J Pharmacol. 145(2): 166-177). New TGF-beta inhibitors can therefore be easily identified by a person skilled in the art.

A TGF-beta inhibitor according to the present invention may be a protein, peptide, small-molecules, small-interfering RNA, antisense oligonucleotide, aptamer or antibody. The inhibitor may be naturally occurring or synthetic. In one embodiment, the TGF-beta inhibitor is an inhibitor of ALK4, ALK5 and/or ALK7. For example, the TGF-beta inhibitor may bind to and directly inhibit ALK4, ALK5 and/or ALK7. Examples of preferred small-molecule TGF-beta inhibitors that can be used in the context of this invention include but are not limited to the small molecule inhibitors listed in table 2 below.

TABLE 2
Small-molecule TGF-beta inhibitors targeting receptor kinases

| | | IC50 | | | |
|---|---|---|---|---|---|
| Inhibitor | Targets | (nM) | Mol Wt | Name | Formula |
| A83-01 | ALK5 | 12 | 421.52 | 3-(6-Methyl-2- | C25H19N5S |
| | (TGF-βR1) | | | pyridinyl)-N- | |
| | ALK4 | 45 | | phenyl-4-(4- | |
| | | | | quinolinyl)-1H- | |
| | ALK7 | 7.5 | | pyrazole-1- | |
| | | | | carbothioamide | |
| SB- | ALK5 | 94 | 384.39 | 4-[4-(1,3- | C22H16N4O3 |
| 431542 | ALK4 | | | benzodioxol-5-yl)- | |
| | ALK7 | | | 5-(2-pyridinyl)-1H- | |
| | | | | imidazol-2- | |
| | | | | yl]benzamide | |
| SB- | ALK5 | 47 | 335.4 | 2-(5- | C20H21N3O2 |
| 505124 | | | | benzo[1,3]dioxol-5- | |
| | | | | yl-2-tert-butyl- | |
| | | | | 3Himidazol- | |
| | ALK4 | 129 | | 4-yl)-6- | |
| | | | | methylpyridine | |
| | | | | hydrochloride | |
| | | | | hydrate | |
| SB- | ALK5 | 14.3 | 343.42 | 6-[2-(1,1- | C21H21N5 |
| 525334 | | | | Dimethylethyl)-5- | |
| | | | | (6-methyl-2- | |
| | | | | pyridinyl)-1H- | |
| | | | | imidazol-4- | |
| | | | | yl]quinoxaline | |
| SD-208 | ALK5 | 49 | 352.75 | 2-(5-Chloro-2- | C17H10ClFN6 |
| | | | | fluorophenyl)-4-[(4- | |
| | | | | pyridyl)amino]pteridine | |
| LY-36494 | TGR-βRI | 59 | 272.31 | 4-[3-(2-Pyridinyl)- | C17H12N4 |
| | TGF-βRII | 400 | | 1H-pyrazol-4-yl]- | |
| | MLK-7K | 1400 | | quinoline | |
| SJN-2511 | ALK5 | 23 | 287.32 | 2-(3-(6- | C17H13N5 |
| | | | | Methylpyridine-2- | |
| | | | | yl)-1H-pyrazol-4- | |
| | | | | yl)-1,5- | |
| | | | | naphthyridine | |

In some embodiments, the TGF-beta inhibitor is a small molecule inhibitor optionally selected from the group consisting of: A83-01, SB-431542, SB-505124, SB-525334, LY 364947, SD-208 and SJN 2511.

In some embodiments, no more than one TGF beta inhibitor is present in the expansion or differentiation medium. In other embodiments, more than one TGF beta inhibitor is present in the expansion or differentiation medium, e.g. 2, 3, 4 or more. In some embodiments, an expansion or differentiation medium of the invention comprises one or more of any of the inhibitors listed in table 2. An expansion or differentiation medium may comprise any combination of one inhibitor with another inhibitor listed. For example, a medium may comprise SB-525334 or SD-208 or A83-01; or SD-208 and A83-01. The skilled person will appreciate that a number of other small-molecule inhibitors exist that are primarily designed to target other kinases, but at high concentrations may also inhibit TGF-beta receptor kinases. For example, SB-203580 is a p38 MAP kinase inhibitor that, at high concentrations (for example, approximate 10 μM or more) is thought to inhibit ALK5. Any such inhibitor that inhibits the TGF-beta signalling pathway can also be used in the context of this invention.

In some embodiments, the TGF-beta inhibitor (e.g. A83-01) is present in the expansion or differentiation medium at least 1 nM, for example, at least 5 nM, at least 50 nM, at least 100 nM, at least 300 nM, at least 450 nM or at least 475 nM. For example, the TGF-beta inhibitor (e.g. A83-01) is present in the expansion or differentiation medium at 1 nM-200 μM, 10 nM-200 μM, 100 nM-200 μM, 1 μM-200 μM, 10 nM-100 μM, 50 nM-100 μM, 50 nM-10 μM, 100 nM-1 μM, 200 nM-800 nM, 350-650 nM or at about 500 nM. Accordingly, in some embodiments, the expansion or differentiation medium comprises A83-01 at a concentration of about 500 nM.

### Gastrin

In some embodiments, the expansion or differentiation medium of the invention further comprises gastrin. In some embodiments, the differentiation medium of the invention comprises gastrin at a concentration of 0.01-500 nM, 0.1-100 nM, 1-100 nM, 1-20 nM or 5-15 nM. For example, in some embodiments, the expansion or differentiation medium of the invention comprises gastrin at a concentration of about 5 nM.

### Supplements

The expansion and differentiation media of the invention are preferably supplemented with one or more (e.g. 1, 2, 3 or all) of the compounds selected from the group consisting of B27, N-acetylcysteine and N2. Thus in some embodiments the medium further comprises one or more components selected from the group consisting of: B27, N2 and N-Acetylcysteine. For example, in some embodiments, the medium further comprises B27, N-Acetylcysteine and N2. In preferred embodiments, the medium further comprises B27 and N-Acetylcysteine.

B27 (Invitrogen), N-Acetylcysteine (Sigma) and N2 (Invitrogen), and Nicotinamide (Sigma) are believed to control proliferation of the cells and assist with DNA stability.

In some embodiments, N-Acetylcysteine is present in the differentiation medium at a concentration of 0.1-200 mM, 0.1-100 mM, 0.1-50 mM, 0.1-10 mM, 0.1-5 mM, 0.5-200 mM, 0.5-100 mM, 0.5-50 mM, 0.5-10 mM, 0.5-5 mM, 1-100 mM, 1-50 mM, 1-10 mM, 1-5 mM. In some embodiments, N-Acetylcysteine is present in the differentiation medium at a concentration of about 1.25 mM.

In some embodiments, the B27 supplement is ‘B27 Supplement minus Vitamin A’ (also referred to herein as “B27 without Vitamin A” or “B27 wo VitA”; available from Invitrogen, Carlsbad, CA; www.invitrogen.com; currently catalog no. 12587010; and from PAA Laboratories GmbH, Pasching, Austria; www.paa.com; catalog no. F01-002; Brewer et al. (1993) J Neurosci Res. 35(5):567-76). In some embodiments, the B27 supplement can be replaced with a generic formulation that comprises one or more of the components selected from the list: biotin, cholesterol, linoleic acid, linolenic acid, progesterone, putrescine, retinyl acetate, sodium selenite, tri-iodothyronine (T3), DL-alpha tocopherol (vitamin E), albumin, insulin and transferrin.

The B27 Supplement supplied by PAA Laboratories GmbH comes as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists. B27 Supplement may be added to a differentiation medium as a concentrate or diluted before addition to a differentiation medium. It may be used at a 1× final concentration or at other final concentrations (e.g. 0.1× to 4× concentration, 0.1× to 2× concentration, 0.5× to 2× concentration, 1× to 4× concentration, or 1× to 2× concentration). Use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into a differentiation medium of the invention. It is also envisaged that some or all of these components may be added separately to the differentiation medium instead of using the B27 Supplement. Thus, the differentiation medium may comprise some or all of these components.

In some embodiments, retinoic acid is absent from the B27 Supplement used in the differentiation medium, and/or is absent from the differentiation medium.

‘N2 Supplement’ (also referred to herein as “N2”) is available from Invitrogen, Carlsbad, CA; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979. The N2 Supplement supplied by PAA Laboratories GmbH comes as a 100× liquid concentrate, containing 500 μg/ml human transferrin, 500 μg/ml bovine insulin, 0.63 μg/ml progesterone, 1611 μg/ml putrescine, and 0.52 μg/ml sodium selenite. N2 Supplement may be added to a differentiation medium as a concentrate or diluted before addition to a differentiation medium. It may be used at a 1× final concentration or at other final concentrations (e.g. 0.1× to 4× concentration, 0.1× to 2× concentration, 0.5× to 2× concentration, 1× to 4× concentration, or 1× to 2× concentration). Use of N2 Supplement is a convenient way to incorporate transferrin, insulin, progesterone, putrescine and sodium selenite into a differentiation medium of the invention. It is of course also envisaged that some or all of these components may be added separately to the differentiation medium instead of using the N2 Supplement. Thus, the differentiation medium may comprise some or all of these components.

In some embodiments in which the medium comprises B27, it does not also comprise N2. The embodiments of the present invention can therefore be adapted to exclude N2 when B27 is present, if desired. In some embodiments N2 is not present in the medium. In some embodiments in which the medium comprises N2, it does not also comprise B27. The embodiments of the present invention can therefore be adapted to exclude B27 when N2 is present, if desired. In some embodiments B27 is not present in the medium. In some embodiments the expansion or differentiation medium is supplemented with B27 and/or N2.

In some embodiments, the basal medium is supplemented with 1-3 mM N-Acetylcysteine; preferably, the basal medium is supplemented with about 1.25 mM N-Acetylcysteine.

Any suitable pH may be used. For example, the pH of the medium may be in the range from about 7.0 to 7.8, in the range from about 7.2 to 7.6, or about 7.4. The pH may be maintained using a buffer. A suitable buffer can readily be selected by the skilled person. Buffers that may be used include carbonate buffers (e.g. NaHCO₃), and phosphates (e.g. NaH₂PO₄). These buffers are generally used at about 50 to about 500 mg/l. Other buffers such as N-[2-hydroxyethyl]-piperazine-N′-[2-ethanesul-phonic acid] (HEPES) and 3-[N-morpholino]-propanesulfonic acid (MOPS) may also be used, normally at around 1000 to around 10,000 mg/1. In some embodiments, the buffer is selected from one or more of the list: phosphate buffer (e.g. KH₂PO₄, K₂HPO₄, Na₂HPO₄, NaCl, NaH₂PO₄) acetate buffer (e.g. HOAc or NaOAc), citrate buffer (e.g. Citric acid or Na-citrate), or a TRIS buffer (e.g. TRIS, TRIS-HCl) or an organic buffer. In some embodiments, the organic buffer is a zwitterionic buffer, such as a Good's buffer, e.g. selected from HEPES, MOPS, MES, ADA, PIPES, ACES, MOPSO, Cholamine Chloride, BES, TES, DIPSO, acetamindoglycine, TAPSO, POPSO, HEPPSO, HEPPS, Tricine, Glycinamide, Bicine, TAPS, AMPSO, CABS, CHES, CAPS and CAPSO. A preferred buffer is HEPES, e.g. at a concentration of 0.1-100 mM, 0.1-50 mM, 0.5-50 mM, 1-50 mM, 1-20 mM or 5-15 mM. In some embodiments, HEPES is added to the culture medium at about 10 mM. A differentiation medium may also comprise a pH indicator, such as phenol red, to enable the pH status of the medium to be easily monitored (e.g. at about 5 to about 50 mg/litre).

An expansion or differentiation medium for use in the invention may comprise one or more amino acids. The skilled person understands the appropriate types and amounts of amino acids for use in differentiation media. Amino acids which may be present include L-alanine, L-arginine, L-asparagine, L-aspartic acid, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine and combinations thereof. Some differentiation media will contain all of these amino acids. Generally, each amino acid when present is present at about 0.001 to about 1 g/L of medium (usually at about 0.01 to about 0.15 g/L), except for L-glutamine which is present at about 0.05 to about 1 g/L (usually about 0.1 to about 0.75 g/L). The amino acids may be of synthetic origin.

An expansion or differentiation medium for use in the invention may comprise one or more vitamins. The skilled person understands the appropriate types and amounts of vitamins for use in differentiation media. Vitamins which may be present include thiamine (vitamin Bi), riboflavin (vitamin B2), niacin (vitamin B3), D-calcium pantothenate (vitamin B5), pyridoxal/pyridoxamine/pyridoxine (vitamin B6), folic acid (vitamin B9), cyanocobalamin (vitamin B12), ascorbic acid (vitamin C), calciferol (vitamin D2), DL-alpha tocopherol (vitamin E), biotin (vitamin H) and menadione (vitamin K).

An expansion or differentiation medium for use in the invention may comprise one or more inorganic salts. The skilled person understands the appropriate types and amounts of inorganic salts for use in differentiation media. Inorganic salts are typically included in differentiation media to aid maintenance of the osmotic balance of the cells and to help 5 regulate membrane potential. Inorganic salts which may be present include salts of calcium, copper, iron, magnesium, potassium, sodium, zinc. The salts are normally used in the form of chlorides, phosphates, sulphates, nitrates and bicarbonates. Specific salts that may be used include CaCl₂), CuSO₄-5H₂O, Fe(NO₃)-9H₂O, FeSO₄-7H₂O, MgCl, MgSO₄, KCl, NaHCO₃, NaCl, Na₂HPO₄, Na₂HPO₄—H₂O and ZnSO₄-7H₂O.

The osmolarity of the medium may be in the range from about 200 to about 400 mOsm/kg, in the range from about 290 to about 350 mOsm/kg, or in the range from about 280 to about 310 mOsm/kg. The osmolarity of the medium may be less than about 300 mOsm/kg (e.g. about 280 mOsm/kg).

An expansion or differentiation medium for use in the invention may comprise a carbon energy source, in the form of one or more sugars. The skilled person understands the appropriate types and amounts of sugars to use in differentiation media. Sugars which may be present include glucose, galactose, maltose and fructose. The sugar is preferably glucose, particularly D-glucose (dextrose). A carbon energy source will normally be present at between about 1 and about 10 g/L.

An expansion or differentiation medium of the invention may contain serum. Serum obtained from any appropriate source may be used, including fetal bovine serum (FBS), goat serum or human serum. Preferably, human serum is used. Serum may be used at between about 1% and about 30% by volume of the medium, according to conventional techniques.

In other embodiments, an expansion or differentiation medium of the invention may contain a serum replacement. Various different serum replacement formulations are commercially available and are known to the skilled person. Where a serum replacement is used, it may be used at between about 1% and about 30% by volume of the medium, according to conventional techniques.

In other embodiments, an expansion or differentiation medium of the invention may be serum-free and/or serum replacement-free. A serum-free medium is one that contains no animal serum of any type. Serum-free media may be preferred to avoid possible xeno-contamination of the stem cells. A serum replacement-free medium is one that has not been supplemented with any commercial serum replacement formulation.

In a preferred embodiment, the expansion or differentiation medium is supplemented with a purified, natural, semi-synthetic and/or synthetic growth factor and does not comprise an undefined component, such as fetal bovine serum or fetal calf serum. For example, supplements such as B27 (Invitrogen), N-Acetylcysteine (Sigma) and N2 (Invitrogen) stimulate proliferation of some cells. In some embodiments, the differentiation medium is supplemented with one or more of these supplements, for example one, any two or all three of these supplements.

An expansion or differentiation medium for use in the invention may comprise one or more trace elements, such as ions of barium, bromium, cobalt, iodine, manganese, chromium, copper, nickel, selenium, vanadium, titanium, germanium, molybdenum, silicon, iron, fluorine, silver, rubidium, tin, zirconium, cadmium, zinc and/or aluminium.

The medium may comprise a reducing agent, such as beta-mercaptoethanol at a concentration of about 0.1 mM.

An expansion or differentiation medium of the invention may comprise one or more additional agents, such as nutrients or growth factors previously reported to improve stem cell culture, such as cholesterol/transferrin/albumin/insulin/progesterone, putrescine, selenite/other factors.

### Exemplary Differentiation Media

Exemplary differentiation media suitable for use with the invention are summarised in Table 3. These differentiation media are particularly suitable for use with organoid-derived monolayers derived from the intestine. A differentiation medium may be selected in order promote the presence or enrichment of specific cell types in the monolayer, for example of one or more cell types listed in Table 3.

TABLE 3
Exemplary differentiation media for intestinal organoid-derived monolayers

| | Medium | | Additional components | | |
|---|---|---|---|---|---|
| Cell type | designation | Composition | iWnt | iNotch | iEGFR |
| Enterocytes | eDM or | ENR | + | | |
| | eCDM | | | | |
| Enteroendocrine cells | endoDM | ENR | + | + | + |
| Goblet cells | gDM | ENR | + | + | |
| Paneth cells | pDM | WENR | | + | |
| Heterogenous | cDM or | WENR or | | + | + |
| combination of | cCDM | 10% Wnt- | | | |
| intestinal epithelial | | conditioned | | | |
| cells | | medium + ENR | | | |

W: Wnt3a or NGS-Wnt;
E: EGF;
N: Noggin;
R: Rspondin 3;
i: Inhibition of the pathway involved

The inventors have shown that culturing organoid-derived monolayers in cDM (also referred to herein as cCDM) results in a higher TEER values and a more heterogeneous cellular composition than those achieved with other differentiation media. Accordingly, in preferred embodiments, the differentiation medium comprises a Notch inhibitor, an EGFR pathway inhibitor and a Wnt agonist.

In some embodiments, the differentiation medium comprises a gamma secretase inhibitor (e.g. DAPT, dibenzazepine (DBZ), benzodiazepine (BZ) or LY-411575), an inhibitor of the RAS-RAF-MAPK pathway (e.g. a MEK inhibitor, such as PD0325901), and one or more Wnt agonists selected from the group consisting of: Rspondin, Wnt conditioned medium and Wnt surrogate.

In a preferred embodiment, the differentiation medium comprises DAPT (e.g. at a concentration of about 10 μM), PD0325901 (e.g. at a concentration of about 100 nM), Wnt-conditioned medium (e.g. at about 10% final volume) and Rspondin (e.g. at a concentration of about 250 ng/mL). In another preferred embodiment, the differentiation medium comprises DAPT (e.g. at a concentration of about 10 μM), PD0325901 (e.g. at a concentration of about 100 nM), Wnt surrogate (e.g. NGS-Wnt at a concentration of about 0.1 nM) and Rspondin (e.g. at a concentration of about 250 ng/mL).

In some embodiments, the differentiation medium comprises a Wnt agonist and an inhibitor of Wnt secretion. In some embodiments, the differentiation medium comprises Rspondin and a Porc inhibitor (e.g. IWP 2, LGK974 or IWP 1). For example, in some embodiments, the differentiation medium comprises Rspondin (e.g. at a concentration of about 250 ng/mL), and IWP 2 (e.g. at a concentration of about 1.5 μM).

In some embodiments, the differentiation medium comprises a Notch inhibitor and a Wnt inhibitor. In some embodiments, the differentiation medium comprises a gamma secretase inhibitor (e.g. DAPT, dibenzazepine (DBZ), benzodiazepine (BZ) or LY-411575) and an inhibitor of Wnt secretion such as a Porc inhibitor (e.g. IWP 2, LGK974 or IWP 1). For example, in some embodiments, the differentiation medium comprises DAPT (e.g. at a concentration of about 10 μM) and IWP 2 (e.g. at a concentration of about 1.5 μM).

In some embodiments, the differentiation medium comprises a Wnt agonist and a Notch inhibitor. In some embodiments, the differentiation medium comprises one or more Wnt agonists selected from the group consisting of: Rspondin, Wnt conditioned medium and Wnt surrogate, and a gamma secretase inhibitor (e.g. DAPT, dibenzazepine (DBZ), benzodiazepine (BZ) or LY-411575). For example, in some embodiments, the differentiation medium comprises Rspondin (e.g. at a concentration of about 250 ng/mL), Wnt-conditioned medium (eg. at about 50% final volume) and DAPT (e.g. at a concentration of about 10 μM). In other embodiments, the medium comprises medium comprises Rspondin (e.g. at a concentration of about 250 ng/mL), Wnt surrogate (e.g. NGS-Wnt at a concentration of about 0.1 nM) and DAPT (e.g. at a concentration of about 10 μM).

In some embodiments, the differentiation medium comprises a Wnt inhibitor, a Notch inhibitor and an EGFR pathway inhibitor. In some embodiments, the differentiation medium comprises an inhibitor of Wnt secretion such as a Porc inhibitor (e.g. IWP 2, LGK974 or IWP 1), a gamma secretase inhibitor (e.g. DAPT, dibenzazepine (DBZ), benzodiazepine (BZ) or LY-411575) and an EGFR pathway inhibitor selected from: an EGFR inhibitor, an EGFR and ErbB2 inhibitor and an inhibitor of the RAS-RAF-MAPK pathway, e.g. a MEK inhibitor such as PD0325901. For example, in some embodiments, the medium comprises IWP-2 (e.g. at a concentration of about 1.5 μM), DAPT (e.g. at a concentration of about 10 μM) and PD0325901 (e.g. at a concentration of about 100 nM).

In any of the above embodiments, the differentiation medium may further comprise a TGF-beta inhibitor, gastrin, a BMP inhibitor and/or a receptor tyrosine kinase ligand. For example, in some embodiments, the differentiation medium further comprises A83-01 (e.g. at a concentration of about 500 nM), gastrin (e.g. at a concentration of about 5 nM), Noggin (e.g. at a concentration of about 100 ng/mL) and EGF (e.g. at a concentration of about 50 ng/mL). In other embodiments, the differentiation medium further comprises A83-01 (e.g. at a concentration of about 500 nM), gastrin (e.g. at a concentration of about 5 nM), Noggin (e.g. at a concentration of about 100 ng/mL) and EGF (e.g. at a concentration of about 50 ng/mL)

Preferably, the differentiation medium does not comprise a p38 MAPK inhibitor (e.g. SB202190) or nicotinamide.

In some embodiments, particularly where the organoids are derived from the lung, the differentiation medium comprises one or more receptor tyrosine kinases, a Wnt agonist, a Notch inhibitor and a BMP pathway activator. In some embodiments, the differentiation medium further comprises a Rho kinase inhibitor and a p38 inhibitor. In a preferred embodiment, the differentiation medium comprises (i) FGF (e.g. FGF-7 at a concentration of about 25 ng/mL and FGF-10 at a concentration of about 100 ng/mL); (ii) Rspondin (e.g. Rspondin-3 at a concentration of about 250 ng/mL); (iii) a Notch inhibitor (e.g. DAPT at a concentration of about 10 μM); (iv) BMP (e.g. BMP4 at a concentration of about 10 ng/mL); (v) a Rho-kinase inhibitor (e.g. Y-27632 at a concentration of about 5 μM); and (vi) a p38 kinase inhibitor (e.g. SB202190 at a concentration of about 500 nM). The lung differentiation medium used herein was previously described in van de Vaart et al. (EMBO reports (2021) 22: e52058).

The invention also provides a differentiation medium which comprises a Notch inhibitor, an EGFR pathway inhibitor and a Wnt agonist, for example according to any of the embodiments described herein.

### Culture Vessels and Extracellular Matrix

Organoid-derived monolayers of the invention are cultured on semi-permeable membranes. Any suitable culture vessel or system comprising a semi-permeable membrane may be used, for example, Transwell® 96 well permeable supports (Corning®). The semi-permeable membrane divides the culture vessel or system into the apical and basolateral compartments. In some embodiments, culture medium is present in both the apical and basolateral compartments. In some embodiments, the apical and basolateral compartments contain the same medium, for example, the same expansion medium or the same differentiation medium. In other embodiments, the medium in the apical compartment is different from that which is present in the basolateral compartment.

In some embodiments, the method of obtaining an organoid-derived monolayer of the invention comprises removing the expansion or differentiation from the apical compartment. Such methods are known in the art as “air-liquid interface” cultures, or ALI cultures, and are particularly suitable for culturing lung organoid-derived monolayers, as shown in the examples.

The culture medium can be refreshed, that is, removed and replenished as and when required. In some embodiments, the culture medium is refreshed every 1, 2, 3, 4, 5, 6 or 7 days. Preferably, the culture medium in both the apical and the basolateral compartments is refreshed every 2-3 days. The culture medium is preferably first removed from the basolateral compartment and subsequently removed from the apical compartment, followed by addition of the culture medium to the apical compartment and subsequent addition of the culture medium to the basolateral compartment. If components are “added” or “removed” from the media, then this can in some embodiments mean that the media itself is removed from the apical and/or basolateral compartments and then a new media containing the “added” component or with the “removed” component excluded is placed in the apical and/or basolateral compartments.

In some embodiments, the organoid-derived monolayer of the invention is cultured in contact with an extracellular matrix (ECM). For example, the semi-permeable membrane may be coated with an ECM. In some embodiments, the semi-permeable membrane is coated with an ECM by adding ECM to the apical compartment and incubating the membrane for an amount of time, e.g. about 30 minutes, about 1 hour, about 2 hours or more, and subsequently seeded with a suspension of organoid-derived single cells and/or organoid fragments. In some embodiments, particularly where the ECM is Matrigel™, the semi-permeable membranes are coated with a concentration of about 2.5% ECM for about 1 hour.

Any suitable ECM may be used. Organoid-derived monolayers are preferably cultured in a microenvironment that mimics at least in part a cellular niche in which its constituent cells naturally reside. A cellular niche is in part determined by the cells and by an ECM that is secreted by the cells in said niche. A cellular niche may be mimicked by culturing organoid-derived monolayers in the presence of biomaterials or synthetic materials that provide interaction with cellular membrane proteins, such as integrins. An ECM as described herein is thus any biomaterial or synthetic material or combination thereof that mimics the in vivo cellular niche, e.g. by interacting with cellular membrane proteins, such as integrins.

In a preferred method of the invention, the organoid-derived monolayer is cultured in contact with an ECM. “In contact” means a physical or mechanical or chemical contact, which means that for separating said organoid-derived monolayer from said extracellular matrix a force needs to be used. In some embodiments, the organoid-derived monolayer is attached to an ECM. A culture medium of the invention may be diffused into a three-dimensional ECM.

One type of ECM is secreted by epithelial cells, endothelial cells, parietal endoderm-like cells (e.g. Englebreth-Holm-Swarm Parietal Endoderm-Like cells described in Hayashi et al. (2004) Matrix Biology 23:47-62) and connective tissue cells. This ECM comprises of a variety of polysaccharides, water, elastin, and glycoproteins, wherein the glycoproteins comprise collagen, entactin (nidogen), fibronectin, and laminin. Therefore, in some embodiments, the ECM for use in the methods of the invention comprises one or more of the components selected from the list: polysaccharides, elastin, and glycoproteins, e.g. wherein the glycoproteins comprise collagen, entactin (nidogen), fibronectin, and/or laminin. For example, in some embodiments, collagen is used as the ECM. Different types of ECM are known, comprising different compositions including different types of glycoproteins and/or different combination of glycoproteins.

The ECM can be provided by culturing ECM-producing cells, such as for example epithelial cells, endothelial cells, parietal endoderm-like cells or fibroblast cells, in a receptacle, prior to the removal of these cells and the addition of a suspension of cells and/or organoid fragments obtained by digesting or dissociating one or more organoids. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C. These are “naturally-produced ECMs”. Naturally-produced ECMs can be commercially provided. Examples of commercially available extracellular matrices include: extracellular matrix proteins (Invitrogen) and basement membrane preparations from Engelbreth-Holm-Swarm (EHS) mouse sarcoma cells (e.g. Cultrex® Basement Membrane Extract (Trevigen, Inc.) or Matrigel™ (BD Biosciences)).

Therefore, in some embodiments, the ECM is a naturally-produced ECM. In some embodiments, the ECM is a laminin-containing ECM such as Matrigel™ (BD Biosciences). In some embodiments, the ECM is Matrigel™ (BD Biosciences), which comprises laminin, entactin, and collagen IV. In some embodiments, the ECM comprises laminin, entactin, collagen IV and heparin sulphate proteoglycan (e.g. Cultrex® Basement Membrane Extract Type 2 (Trevigen, Inc.)). In some embodiments, the ECM comprises at least one glycoprotein, such as collagen and/or laminin. A preferred ECM for use in a method of the invention comprises collagen and laminin. A further preferred ECM comprises laminin, entactin, and collagen IV. Mixtures of naturally-produced or synthetic ECM materials may be used, if desired.

In another embodiment, the ECM may be a synthetic ECM. For instance, a synthetic ECM, such as ProNectin (Sigma Z378666) may be used. In a further example, the ECM may be a plastic, e.g. a polyester, or a hydrogel. In some embodiments, a synthetic matrix may be coated with biomaterials, e.g. one or more glycoprotein, such as collagen or laminin.

In some embodiments, the expansion or differentiation medium further comprises an integrin agonist (e.g. as described in WO2020/234250). Specific examples of integrin agonists include anti-integrin antibodies, such as anti-bI integrin antibodies (e.g. TS2/16, 12G10, 8A2, 15/7, HUTS-4, 8E3, N29 and 9EG7 antibodies). The integrin agonist may be used instead of or in addition to the extracellular matrix.

### Co-Culture

In some embodiments, the monolayer contains epithelial cells in co-culture with non-epithelial cells. In other embodiments, the monolayer contains only epithelial cell types. Methods of co-culturing organoids and immune cells are described in WO2019/122388. Co-cultures of organoid-derived monolayers and immune cells may be useful for investigating the physiology of diseases and/or the suitability (efficacy and/or safety) of candidate agents for treating diseases. Accordingly, in some embodiments, the organoid-derived monolayer of the invention is co-cultured with immune cells.

### Properties of Organoid-Derived Monolayers of the Invention

In some embodiments, the organoid-derived monolayer of the invention has TEER of about 10, about 50, about 100, about 200, about 300, about 400, about 500, about 600, about 700, about 800, about 900, about 1000, about 1100, about 1200, about 1300, about 1400 or about 1500 Ω·cm². In some embodiments, the organoid-derived monolayer of the invention has TEER of about 2, about 5, about 10, about 50, about 100, about 200, about 300, about 400, about 500, about 600, about 700, about 800, about 900, about 1000, about 1100, about 1200, about 1300, about 1400 or about 1500 Ω·cm².

In some embodiments, the organoid-derived monolayer of the invention has TEER of about 100, about 200, about 300, about 400, about 500, about 600, about 700, about 800, about 900, about 1000, about 1100, about 1200, about 1300, about 1400 or about 1500 Ω·cm². Preferably, the organoid-derived monolayer of the invention has TEER of more than 100 Ω·cm².

In some embodiments, particularly where the organoid-derived monolayer is derived from the kidney, the monolayer has TEER of more than 25, more than 50, more than 75, more than 100, more than 200, more than 300, more than 400, more than 500, more than 600, more than 700, more than 800, more than 900, more than 1000, more than 1100, more than 1200, more than 1300 or more than 1400 Ω·cm².

In some embodiments, the organoid-derived monolayer of the invention, e.g. derived from intestinal organoids, comprises one or more of the following cell types: Lgr5+ stem cell, enterocyte, goblet cell, Paneth cell and enteroendocrine cell. Cellular composition of the organoid-derived monolayer may be evaluated by detecting or quantifying the expression of one or more marker genes. Lgr5 is a marker for Lgr5⁺ stem cells. Ki67 is a marker for proliferating cells, such as Lgr5⁺ stem cells. Goblet cells may be detected by staining for mucus, e.g. by performing Alcian blue staining, or detecting the expression of mucin-2 (Muc2), as described herein. Intestinal alkaline phosphatase (ALPI or ALPI1) is a marker for enterocytes. Lysozyme is a marker for Paneth cells. Chromogranin A is a marker for enteroendocrine cells.

In some embodiments, the organoid-derived monolayer of the invention, e.g. derived from lung organoids, comprises one or more (preferably all) of the following cell types: club cells, basal cells, ciliated cells, goblet cells, alveolar type I cells and alveolar type II cells. In some embodiments, the organoid-derived monolayer of the invention, e.g. derived from lung organoids, expresses one or more (preferably all) of the following genes which are markers for particular cell types: KRT5 (lung basal cell marker), SPDEF (goblet cell marker), FOXJ1 (ciliated cell marker), and SFTPA1 (lung alveoli marker, particularly for type II alveolar cells).

In some embodiments, the organoid-derived monolayer of the invention, e.g. derived from kidney organoids, comprises one or more (preferably all) of the following cell types: proximal tubule cells, kidney epithelial cells, loop of Henle cells, distal tubule cells and collecting duct cells. ABCC4 is a proximal tubule marker, PAX8 is a kidney epithelial marker, CLDN10 is a loop of Henle marker, SLC12A3 is distal tubule marker, and AQP3 is a collecting duct marker.

Depending on the identity of the marker, the expression of said marker may be assessed by RT-PCR, immuno-histochemistry or histological staining after 3 days, 4 days, 5 days, 6 days, 7 days, 8 days, 9 or more of culture in an expansion or differentiation medium, as described herein. In some embodiments, the expression of the marker is measured after 3 days, 4 days, 5 days, 6 days, 7 days, 8 days, 9 days, 10 days, 11 days, 12 days or more, e.g. 16 days, of culture in an expansion and/or differentiation medium, as described herein.

The term “expressed” is used to describe the presence of a marker within a cell. In order to be considered as being expressed, a marker must be present at a detectable level. By “detectable level” is meant that the marker can be detected using one of the standard laboratory methodologies such as PCR, blotting or FACS analysis. A gene is considered to be expressed by a cell of the population of the invention if expression can be reasonably detected after 30 PCR cycles, which corresponds to an expression level in the cell of at least about 100 copies per cell. The terms “express” and “expression” have corresponding meanings. At an expression level below this threshold, a marker is considered not to be expressed. The comparison between the expression level of a marker in a cell of the invention, and the expression level of the same marker in another cell, such as for example an embryonic stem cell, may preferably be conducted by comparing the two cell types that have been isolated from the same species. Preferably this species is a mammal, and more preferably this species is human. Such comparison may conveniently be conducted using a reverse transcriptase polymerase chain reaction (RT-PCR) experiment.

In some embodiments, the organoid-derived monolayer of the invention expresses one or more of the following markers: ALPI, MUC2, lysozyme, Ki67 and Lgr5. In some embodiments, the organoid-derived monolayer of the invention expresses Lgr5 and Muc2. In some embodiments, the organoid-derived monolayer of the invention does not express ALPI. In some embodiments, the organoid-derived monolayer of the invention expresses Lgr5 and Muc2 and does not express ALPI. In some embodiments, the organoid-derived monolayer of the invention expresses lysozyme.

In some embodiments, particularly where the monolayers are derived from kidney organoids, the organoid-derived monolayer of the invention expresses one or more, preferably all of ABCC4, PAX8, CLDN10, SLC12A3, AQP3, OCT2, MATE1 and MATE2-K. In some embodiments, the organoid-derived monolayer expresses one or more, preferably all of PAX8, CLDN10, AQP3, OCT2, MATE1 and MATE2-K. In some embodiments, the organoid-derived monolayer expresses one or more, preferably all of PAX8, OCT2, MATE1 and MATE2-K. In some embodiments, the organoid-derived monolayer does not express OAT1 or OAT3.

In some embodiments, particularly where the monolayers are derived from lung organoids, the organoid-derived monolayer of the invention expresses one or more, preferably all of KRT5, SPDEF, FOXJ1, and SFTPA1. In some embodiments, the organoid-derived monolayer expresses one or more, preferably all of KRT5, SPDEF, and FOXJ1. In some embodiments, the organoid-derived monolayer expresses one or more, preferably all of KRT5, SPDEF, and SFTPA1.

In some embodiments, the organoid-derived monolayer of the invention is polarised along the apical-basal axis. In some embodiments, TEER of the organoid-derived monolayer of the invention decreases and/or permeability to a dye, such as Lucifer yellow, of the organoid-derived monolayer of the invention increases when an EGFR inhibitor, such as Gefitinib, is applied to the basolateral side of the monolayer. In some embodiments, TEER of the organoid-derived monolayer of the invention does not decrease and/or permeability to a dye, such as Lucifer yellow, of the organoid-derived monolayer of the invention does not increase when an EGFR inhibitor, such as Gefitinib, is applied to the apical side of the monolayer.

In some embodiments, the organoid-derived monolayer of the invention is impermeable to a dye, such as Lucifer yellow. In some embodiments, the organoid-derived monolayer of the invention is permeable to a dye, such as Lucifer yellow, when it has been scratched, e.g. with a tip of a pipette.

In some embodiments, the organoid-derived monolayer of the invention has a smooth apical surface. In some embodiments, the organoid-derived monolayer of the invention has an invaginated apical morphology. In some embodiments, the organoid-derived monolayer of the invention has cilia on the apical surface (e.g. see the lung organoid-derived monolayers in FIG. 16C). In some embodiments, particularly where the monolayer is derived from lung organoids, the monolayer has bubble-like structures.

Organoid-derived monolayers may be pseudostratified (e.g. see the lung organoid-derived monolayers in FIGS. 16A and 16C).

In some embodiments, the organoid-derived monolayer of the invention possesses transport function, i.e. it are capable of transporting substrates from the apical to the basolateral compartment or from the basolateral to the apical compartment. Transport function may be determined using the assays described herein.

### Uses of Organoid-Derived Monolayers

Uses of the organoid-derived monolayers described herein are likewise provided. For example, the invention provides the use of an organoid-derived monolayer in a drug discovery screen; toxicity assay; research of tissue embryology, cell lineages, and differentiation pathways; research to identify the chemical and/or neuronal signals that lead to the release of the respective hormones; gene expression studies including recombinant gene expression; research of mechanisms involved in tissue injury and repair; research of inflammatory and infectious diseases; studies of pathogenetic mechanisms; or studies of mechanisms of cell transformation and aetiology of cancer.

The invention provides the use of an organoid-derived monolayer of the invention in drug screening, (drug) target validation, (drug) target discovery, toxicology and toxicology screens, personalized medicine and/or as ex vivo cell/organ models, such as disease models.

Organoid-derived monolayers of the invention are thought to faithfully represent the in vivo situation. Therefore, as well as providing normal ex vivo cell/organ models, the organoids of the invention can be used as ex vivo disease models.

Organoid-derived monolayers of the invention can also be used for culturing of a pathogen and thus can be used as ex vivo infection models. Examples of pathogens that may be cultured using an organoid of the invention include viruses, bacteria, prions or fungi that cause disease in its animal host. Thus an organoid-derived monolayer of the invention can be used as a disease model that represents an infected state. In some embodiments of the invention, the organoids can be used in vaccine development and/or production.

Diseases that can be studied by the organoid-derived monolayers of the invention thus include genetic diseases, metabolic diseases, pathogenic diseases, inflammatory diseases etc., for example including, but not limited to: diabetes (such as type I or type II), cystic fibrosis, carcinomas, adenocarcinomas, adenomas, gastroenteropancreatic neuroendocrine tumours, inflammatory bowel disease (such as Crohn's disease or ulcerative colitis), coeliac disease and leaky gut syndrome.

Traditionally, cell lines and more recently iPS cells have been used as ex vivo cell/organ and/or disease models (for example, see Robinton et al. Nature 481, 295, 2012). However, these methods suffer a number of challenges and disadvantages. For example, cell lines cannot be obtained from all patients (only certain biopsies result in successful cell lines) and therefore, cell lines cannot be used in personalised diagnostics and medicine. iPS cells usually require some level of genetic manipulation to reprogramme the cells into specific cell fates. Alternatively, they are subject to culture conditions that affect karyotypic integrity and so the time in culture must be kept to a minimum (this is also the case for human embryonic stem cells). This means that iPS cells cannot accurately represent the in vivo situation but instead are an attempt to mimic the behaviour of in vivo cells. Cell lines and iPS cells also suffer from genetic instability.

By contrast, the organoid-derived monolayers of the invention provide a genetically stable platform which faithfully represents the in vivo situation. In some embodiments, the organoid-derived monolayers of the invention comprise all differentiated cell types that are present in the corresponding in vivo situation. In other embodiments, the organoid-derived monolayers of the invention may be further differentiated to provide all differentiated cell types that are present in vivo. Thus the organoid-derived monolayers of the invention can be used to gain mechanistic insight into a variety of diseases and therapeutics, to carry out in vitro drug screening, to evaluate potential therapeutics, to identify possible targets (e.g. proteins) for future novel (drug) therapy development and/or to explore gene repair coupled with cell-replacement therapy.

For these reasons, the organoid-derived monolayers of the invention can be a tool for drug screening, target validation, target discovery, toxicology and toxicology screens and personalized medicine.

Accordingly, the invention also provides use of an organoid-derived monolayer of the invention in an assay assessing epithelial viability, metabolic activity, permeability, barrier function integrity and/or activity of transporter proteins. Methods of assessing viability, permeability and barrier function integrity of organoid-derived monolayers and activity of transporter proteins in organoid-derived monolayers are described herein.

The invention also provides a method of identifying a compound capable of modulating epithelial viability, metabolic activity, permeability, barrier function integrity and/or activity of transporter proteins comprising:
i. contacting an organoid-derived monolayer, for example an organoid-derived monolayer as described herein, with one or more candidate molecules; and
ii. assessing the viability, metabolic activity, permeability and/or barrier function integrity of the organoid-derived monolayer and/or activity of transporter proteins in the organoid-derived monolayer.

“Modulating” may be improving, restoring, damaging or inhibiting epithelial viability, metabolic activity, permeability, barrier function integrity and/or activity of transporter proteins.

In some embodiments, the one or more candidate molecules are a library of candidate molecules, or part of a library of candidate molecules.

The invention also provides a method of assessing the effect of a compound on epithelial viability, metabolic activity, permeability, barrier function integrity and/or activity of transporter proteins comprising:
i. contacting an organoid-derived monolayer, for example an organoid-derived monolayer as described herein, with said compound; and
ii. assessing the viability, metabolic activity, permeability and/or barrier function integrity of the organoid-derived monolayer and/or activity of transporter proteins in the organoid-derived monolayer.

In some embodiments, the compound is an approved or experimental drug, for example for a disease or the disorder of the digestive system, such as inflammatory bowel disease (e.g. Crohn's disease or ulcerative colitis), coeliac disease or leaky gut syndrome. In some embodiments, the compound is tofacitinib.

The inventors have shown that epithelial barrier injury may be induced in organoid-derived monolayers using a combination of proinflammatory cytokines, thereby providing a useful model with which to study the effects of compounds on epithelial viability, metabolic activity, permeability, barrier function integrity and/or activity of transporter proteins.

Accordingly, in some embodiments, the methods of assessing the effect of a compound on epithelial viability, metabolic activity, permeability, barrier function integrity and/or activity of transporter proteins, or the methods of identifying a compound capable of modulating epithelial viability, metabolic activity, permeability, barrier function integrity and/or activity of transporter proteins described herein further comprise contacting the organoid-derived monolayer with one or more proinflammatory cytokines, for example selected from the group consisting of: IL-1α, IL-1β IL-2, IL-12, IL-17, IL-18, IFN-γ, and TNF-α. Preferably, the one or more proinflammatory cytokines are selected from selected from the group consisting of: IFN-γ, TNF-α and IL-1α. In some embodiments, the one or more proinflammatory cytokines comprise IFN-γ, TNF-α and IL-1α. In some embodiments, the one or more proinflammatory cytokines comprise IFN-γ and TNF-α. In some embodiments, the one or more proinflammatory cytokines comprise TNF-α and IL-1α. The step of contacting the organoid-derived monolayer with one or more proinflammatory cytokines may be carried out before, after, or simultaneously with the step of contacting the monolayer with said compound or with the one or more candidate molecules. In preferred embodiments, the organoid-derived monolayer is contacted with one or more proinflammatory cytokines after the step of contacting the 10 monolayer with said compound or with the one or more candidate molecule.

The invention also provides a method of identifying a mutation associated with epithelial viability, metabolic activity, permeability, barrier function integrity and/or activity of transporter proteins comprising:
i. assessing the viability, metabolic activity, permeability and/or barrier function integrity of an organoid-derived monolayer and/or activity of transporter proteins in an organoid-derived monolayer, for example an organoid-derived monolayer as described herein; and
ii. determining the presence of one or more mutations in the genome of one or more cells in the organoid-derived monolayer.

Mutations may be identified by sequencing the genome of one or more cells in the organoid-derived monolayer and/or performing a single-nucleotide polymorphism (SNP) microarray on DNA isolated from one or more cells in the organoid-derived monolayer.

The invention also provides a method of diagnosing a disease or affliction that affects epithelial viability, metabolic activity, permeability, barrier function integrity 25 and/or activity of transporter proteins, or determining an increased risk of said disease or affliction, in a human subject comprising:
i. obtaining an organoid-derived monolayer from said human subject using methods described herein; and
ii. testing the viability, metabolic activity, permeability and/or barrier function integrity of the organoid-derived monolayer and/or activity of transporter proteins in the organoid-derived monolayer,
   wherein a test result above or below a reference value indicates the presence of, or an increased risk of, said disease or affliction in the human subject.

In some embodiments, the reference value is a value obtained from a control. In some embodiments, the control is an organoid-derived monolayer obtained from a healthy human subject.

In some embodiments, the disease or affliction is a disease or disorder of the digestive system. In some embodiments, the disease or affliction is inflammatory bowel disease (e.g. Crohn's disease or ulcerative colitis), coeliac disease or leaky gut syndrome. Preferably, the disease or affliction is inflammatory bowel disease (e.g. Crohn's disease or ulcerative colitis).

The invention also provides a method of predicting the likelihood of a patient's response to a candidate compound comprising:
i. obtaining an organoid-derived monolayer from said patient using methods described herein;
ii. contacting the organoid-derived monolayer with said compound; and
iii. assessing the viability, metabolic activity, permeability and/or barrier function integrity of the organoid-derived monolayer and/or activity of transporter proteins in the organoid-derived monolayer.

In some embodiments, the patient has a disease or disorder of the digestive system. In some embodiments, the disease or disorder is inflammatory bowel disease (e.g. Crohn's disease or ulcerative colitis), coeliac disease or leaky gut syndrome. Preferably, the patient has inflammatory bowel disease (e.g. Crohn's disease or ulcerative colitis). In some embodiments, the candidate compound is an approved or experimental drug for any of the above-listed diseases or disorders. In some embodiments, the candidate compound is tofacitinib.

Assessment of Viability, Metabolic Activity, Permeability, Barrier Function Integrity and/or Activity of Transporter Proteins

### Viability

Viability of the organoid-derived monolayers of the invention may be measured using any suitable method, for example, Hoechst staining, Propidium Iodide staining in FACS, or, preferably, an ATP-based assay, for example as described herein.

### Transepithelial Electrical Resistance (TEER)

In some embodiments, the barrier function integrity of the organoid-derived monolayer is assessed by measuring transepithelial electrical resistance (TEER). TEER measurements are widely accepted as a method to analyse tight junction dynamics and barrier function integrity in biological models of physiological barriers, such as epithelial monolayers. Methods of measuring TEER have been described (see Srinivasan, B. et al. TEER measurement techniques for in vitro barrier model systems. Journal of Laboratory Automation. 20 (2), 107-126 (2015), and Blume, L.-F. et al. Temperature corrected transepithelial electrical resistance (TEER) measurement to quantify rapid changes in paracellular permeability. Die Pharmazie. 65 (1), 19-24 (2010)). TEER may be measured using a manual TEER meter or an automated TEER measurement robot.

### Permeability

Permeability may be used as an indication of monolayer integrity. Permeability may be transcellular or paracellular. Paracellular permeability is controlled by tight junctions.

In some embodiments, assessment of permeability of the organoid-derived monolayer comprises measuring the rate of passive diffusion of a reporter compound from the apical to the basolateral side of the monolayer. Any suitable reporter compound may be used. In some embodiments, the reporter compound is a dye. In some embodiments, the reporter compound is a labelled compound, for example a radiolabelled compound, a fluorescently labelled compound, or a compound labelled with a dye. Preferably, the reporter compound is Lucifer yellow. In other embodiments, the reporter compound is dextran, which is optionally labelled with a dye, e.g. a fluorescent dye such as tetramethylrhodamine isothiocyanate (TRITC). In some embodiments, the concentration of the reporter compound in the apical and/or basolateral compartment is measured using mass spectrometry. In some embodiments, the concentration of the reporter compound in the apical and/or basolateral compartment is measured using liquid chromatography-mass spectrometry. In some embodiments, the concentration of the reporter compound in the apical and/or basolateral compartment is measured using colorimetry.

In some embodiments, the rate of passive diffusion of a reporter compound across the monolayer is measured by applying the reporter compound to the apical compartment and measuring the amount of the reporter compound in the basolateral compartment. In other embodiments, the rate of passive diffusion of a reporter compound across the monolayer is measured by applying the reporter compound to the basolateral compartment and measuring the amount of the reporter compound in the apical compartment. The amount of the reporter compound in the apical or basolateral compartment may be measured after a period of incubation, for example, after 1 hour, after 2 hours, after 3 hours, after 4 hours, after 5 hours, after 6 hours, after 7 hours, after 8 hours, after 9 hours, after 10 hours or longer. In some embodiments, the amount of the reporter compound in the apical or basolateral compartment is measured repeatedly, for example, every minute or every hour.

### Activity of Transporter Proteins

Transport function of the organoid-derived monolayers of the invention may be evaluated by assessing the activity of transporter proteins in the monolayer. In some embodiments, assessing the activity of transporter proteins comprises measuring the rate of transport of a substrate of a transporter protein across the monolayer, optionally in the presence of an inhibitor of said transporter protein. In some embodiments, assessing the activity of transporter proteins comprises measuring the rate of transport of a substrate of a transporter protein into the cells of the monolayer, optionally in the presence of an inhibitor of said transporter protein. In some embodiments, the transporter protein is selected from P-glycoprotein 1 (Pgp1, also known as multidrug resistance protein 1 (MDR1) or ATP-binding cassette sub-family B member 1 (ABCB1)), breast cancer resistance protein (BCRP or ABCG2), peptide transporter 1 (PEPT1) and multi-drug resistance protein 2 (MRP2). In some embodiments, the transporter protein is selected from P-glycoprotein 1 (Pgp1, also known as multidrug resistance protein 1 (MDR1) or ATP-binding cassette sub-family B member 1 (ABCB1)), breast cancer resistance protein (BCRP or ABCG2), peptide transporter 1 (PEPT1), multi-drug resistance protein 2 (MRP2), multi-drug resistance protein 1 (MRP1, also known as ABCC1) and organic cation transporter 2 (Oct2, also known as SLC22A2). In some embodiments, the substrate is labelled, for example, fluorescently, with a radioisotope, or with a dye. In some embodiments, the substrate is a dye. In some embodiments, the concentration of the substrate in the apical and/or basolateral compartment is measured using mass spectrometry. In some embodiments, the concentration of the substrate in the apical and/or basolateral compartment is measured using liquid chromatography-mass spectrometry. In some embodiments, the concentration of the substrate in the apical and/or basolateral compartment is measured using colorimetry. In some embodiments, the amount of the substrate that has been transported into the cells of the monolayer (i.e. cellular accumulation) is assessed by measuring intracellular fluorescence.

A summary of exemplary substrates and inhibitors suitable for use with the invention and their target transporter proteins is shown in Table 4.

TABLE 4
Exemplary transporter proteins, and their substrates and inhibitors

| Transporter protein | Substrate(s) | Inhibitor(s) |
|---|---|---|
| Pgp1 | Rhodamine 123 | GF120918 |
| | Digoxin | PSC833 |
| | Calcein AM | |
| MRP2 | Sulfasalazine | MK517 |
| BCRP | Estron-3-sulfate | Ko143 |
| PEPT1 | Valacyclovir | GlySar |
| MRP1 | Calcein AM | MK571 |
| OCT2 | Rhodamine 123 | Decynium-22 |

In a preferred embodiment, the transporter protein is Pgp1, the substrate is Rhodamine 123, and the inhibitor is PSC833. In another preferred embodiment, the transporter protein is Pgp1, the substrate is Calcein AM, and the inhibitor is PSC833. In another preferred embodiment, the transporter protein is MRP1, the substrate is Calcein AM, and the inhibitor is MK571. In another preferred embodiment, the transporter protein is OCT2, the substrate is Rhodamine 123, and the inhibitor is Decynium-22.

In some embodiments, the activity of more than one, e.g. two, transporter proteins is assessed simultaneously. In such embodiments, two or more transporter protein inhibitors may be used.

### Kits

The invention also provides a kit for generating organoid-derived monolayers of the invention comprising organoids and one or more culture media as described herein. In particular, the kit may comprise organoids, an expansion medium and optionally one or more differentiation media as described herein. The kit may further comprise a cell dissociation reagent, ROCK inhibitor, an extracellular matrix, and one or more semi-permeable membranes. In some embodiments, the membranes are provided already pre-coated with the extracellular matrix.

The invention further provides a kit comprising a culture medium, such as an expansion or differentiation medium as described herein. In preferred embodiments, the kit comprises a differentiation medium comprising a Notch inhibitor, an EGFR pathway inhibitor and a Wnt agonist. Exemplary differentiation media comprising a Notch inhibitor, an EGFR pathway inhibitor and a Wnt agonist are described herein.

The invention further provides a kit for preparing a differentiation medium comprising a Notch inhibitor, an EGFR pathway inhibitor and a Wnt agonist. Suitable Notch inhibitors, EGFR pathway inhibitors and Wnt agonists are described herein. In a preferred embodiment, the kit comprises DAPT, PD0325901, Wnt-conditioned medium and Rspondin. In another preferred embodiment, the kit comprises DAPT, PD0325901, Wnt surrogate (e.g. NGS-Wnt) and Rspondin.

The invention further provides a kit for assessing the barrier and transport functions of organoid-derived monolayers comprising one or more of the following components: gefitinib, staurosporin, Lucifer Yellow, Calcein AM, Rhodamine 123, a P-gp inhibitor (e.g. PSC-833), an OCT2 inhibitor (e.g. Decynium-22), tofacitinib and one or more pro-inflammatory cytokines. One or more of these components may also be provided as part of a kit comprising organoids and one or more culture media as described above.

### Definitions

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” or “approximately” means that the value presented can be varied by +/−10%. The value can also be read as the exact value and so the term “about” can be omitted. For example, the term “about 100” encompasses 90-110 and also 100.

The term “digestive system” encompasses the gastrointestinal tract and the liver, pancreas and gallbladder.

The term “gastrointestinal tract” encompasses mouth, esophagus, stomach, intestine and anus.

The term “intestine” encompasses colon and small intestine.

The term “small intestine” encompasses duodenum, jejunum and ileum.

The term “lung” encompasses the trachea, bronchi, bronchioles, alveolar ducts and alveoli.

The term “kidney” encompasses the ureter, cortex, medulla, renal pelvis and calyces.

All patent and literature references cited in the present specification are hereby incorporated by reference in their entirety.

The following examples are offered for illustrative purposes only, and are not intended to limit the scope of the present invention in any way.

### DESCRIPTION OF THE DRAWINGS

Embodiments of the invention will be described, by way of example, with reference to the following drawings, in which:

FIG. 1 illustrates organoid-derived monolayer formation after seeding single cells on membranes. (A) Single cells just after seeding on membranes. On average, (B) the monolayer is around 50% confluent 1-3 days after seeding, (C) ˜90% confluent at day 3-5, and (D) the complete monolayer has formed around day 4-7. Scale bars=100 μm.

FIG. 2 illustrates enrichment of specific cell types in the organoid-derived monolayer. (A) Monolayer after 8 days in IEM. (B) Monolayer enriched with enterocytes after 4 days in IEM and another 4 days in eDM. (C) Monolayer enriched with goblet cells and other cell types after 4 days in IEM and another 4 days in cDM. Scale bars=100 μm. Abbreviations: IEM=intestinal organoid expansion medium; eDM=enterocyte differentiation medium; cDM=combination differentiation medium.

FIG. 3 illustrates a variety of possible readouts using epithelial organoid monolayers. (A) Electrode in the membrane insert to measure TEER. (B) TEER values increase in time with a value of ˜100 Ω·cm² when the monolayer reaches confluence. After enriching monolayers with enterocytes or a combination of different epithelial cells, TEER increases to 1000 Ω·cm² or higher. (C) Monolayers in all medium conditions (IEM+4 days IEM/eDM/cDM) are impermeable to Lucifer Yellow. (D) Expression of lysozyme is higher in ileum monolayers when grown in expansion medium than in either type of differentiation medium (IEM+4 days IEM/eDM/cDM). (E) Colon monolayers show different morphologies when exposed to different medium conditions (IEM+4 days IEM/eDM/cDM) as visualized by H&E, Ki67, Alcian Blue, and MUC2 stains. As expected, monolayers cultured in expansion medium are very proliferative, as shown by Ki67 staining. Monolayers differentiated with eDM show a columnar epithelium without proliferative cells. Monolayers exposed to cDM are also not proliferative and develop more goblet cells. Scale bar=100 μm. (F) Stem cell (LGR5), goblet cell (MUC2), and enterocyte (ALPI) marker gene expression in colon monolayers by qRT-PCR. Abbreviations: TEER=transepithelial electrical resistance; IEM=intestinal organoid expansion medium; eDM=enterocyte differentiation medium; cDM=combination differentiation medium; Pₐₚₚ=apparent permeability coefficient; LGR5=leucine-rich repeat-containing G-protein-coupled receptor 5; H&E=hematoxylin and eosin; AB=Alcian Blue; MUC2=mucin-2; ALPI=intestinal alkaline phosphatase; qRT-PCR=quantitative reverse-transcription polymerase chain reaction.

FIG. 4 illustrates characterization of normal ileum organoid monolayers cultured in expansion CNM (left), enterocyte condition eCDM (middle) and combination cCDM (right) culture conditions on 96 well transwell plates. (A) Ileum organoid monolayers stained with haematoxylin and eosin (H&E), Alcian blue (AB), KI67 and MUC2. Representative images from two independent biological replicates are presented. Scale bars represent 100 μm. (B) Expression of cell-specific genes (i.e. LGR5, MUC2, LYZ and ALPI1) in ileum organoid monolayers from two independent biological replicates in different culture conditions. For every biological replicate, two technical replicates were measured. Data are represented as mean±SD for 2 replicates of 2 independent experiments. (C) Lysozyme activity in apical chamber medium supernatant. (D) Transepithelial electrical epithelial resistance (TEER) and (E) Lucifer yellow (LY) permeability results from ileum organoid monolayers cultured in expansion (CNM), enterocyte (eCDM) and combination (cCDM) conditions. LY permeation was measured at the end of experiment and represented as apparent permeability (Papp). C, D and E are represented as mean±SD for 3 technical replicates.

FIG. 5 illustrates characterization of normal colon organoid monolayers cultured in expansion CNM (left), enterocyte condition eCDM (middle) and combination cCDM (right) on 96 well transwell plates. (A) Colon organoid monolayers stained with haematoxylin and eosin (H&E), KI67, Alcian blue (AC) and MUC2. Representative images from two independent biological replicates are presented. Scale bars represent 100 μm. (B) Expression of cell-specific genes (i.e. LGR5, MUC2, LYZ and ALPI) in colon organoid monolayers from three independent biological replicates in different culture conditions. For every biological replicate, two technical replicates were measured. Data are represented as mean±SD for 2 replicates of 3 independent experiments. (C) Lysozyme activity in apical chamber medium supernatant. (D) Transepithelial electrical epithelial resistance (TEER) and (E) Lucifer yellow (LY) permeability results from colon organoid monolayers cultured in expansion (CNM), enterocyte (eCDM) and combination (cCDM) conditions. LY permeation was measured at the end of experiment and represented as apparent permeability (Papp). C, D and E are represented as mean±SD for 3 technical replicates.

FIG. 6 illustrates inducing barrier injury to normal colon derived epithelium monolayers on 96 well transwell plates in expansion (CNM) and combination (cCDM) culture conditions by serial titration of proinflammatory cytokines. All proinflammatory cytokines were used at the final concentrations listed on the graph regardless of combination. (A)-(H) illustrate transepithelial epithelial resistance (TEER) in CNM and cCDM culture conditions. Data are represented as mean±SD for 3 technical replicates. (I) epithelium monolayers TEER EC50 dose response curve after 24 hours addition of proinflammatory cytokine combination on CNM and (J) cCDM culture conditions. EC50 dose response curves were calculated by Non-linear regression log(inhibitor) versus response variable slope (four parameters). Note that 20 ng/ml data points for IFγ/TNF-α treatment was excluded for EC50 calculation as did not follow the dose response curve decreasing TEER trend in (J).

FIG. 7 illustrates Tofacitinib pre-treatment titration on normal colon-derived epithelium monolayers on 96 well transwell plates treated with 1 ng/ml proinflammatory cytokine combinations IFN-γ/TNF-α/IL-1α (top) and IFN-γ/TNF-α (bottom). (A) Transepithelial electrical resistance (TEER). (B) relative TEER value from the same Transwell before treatment after 5 and 24 hours. (C) Permeability and (D) Cell viability of the same Transwell after 24 hours in response to treatments. Data are represented as mean±SD of 3 technical replicates. (E) TEER (top), permeability (middle) and cell viability (bottom) dose response curves. EC₅₀ values were calculated by Non-linear regression log(inhibitor) versus response variable slope (four parameters) of three technical replicates.

FIG. 8 illustrates that Tofacitinib pre-treatment inhibits proinflammatory cytokine-induced barrier injury in normal colon organoid monolayers on 96 well transwell plates. (A) Transepithelial electrical resistance (TEER). (B) Relative TEER values from the same Transwell before treatment after 5 and 24 hours. (C) Permeability and (D) Cell viability of the same Transwell after 24 hours in response to treatments. Data are represented as mean±SD for 3 technical replicates. An unpaired t-test was performed on permeability and cell viability data. A one-way ANOVA (Dunett's multiple comparisons test) was done on normalized TEER, permeability and cell viability data. *P<0.05, **P<0.01, ***P<0.001.

FIG. 9 illustrates that Tofacitinib pre-treatment inhibits proinflammatory cytokine-induced barrier injury in normal ileum organoid monolayers on 96 well transwell plates. (A) Transepithelial electrical resistance (TEER). (B) Relative TEER values from the same Transwell before treatment after 5 and 24 hours. (C) Permeability and (D) Cell viability of the same Transwell after 24 hours in response to treatments. Data are represented as mean±SD for 3 technical replicates. An unpaired t-test was performed on permeability and cell viability data. A one-way ANOVA (Dunett's multiple comparisons test) was done on normalized TEER, permeability and cell viability data. *P<0.05, **P<0.01, ***P<0.001.

FIG. 10 illustrates uninflamed CD ileum-derived organoids epithelium monolayers response to proinflammatory cytokine combination IFN-γ/TNF-α/IL-1α (top) and IFN-γ/TNF-α (bottom) induced barrier injury with and without tofacitinib pre-treatment. (A) Transepithelial electrical resistance (TEER). (B) relative TEER value from the same transwell before treatment after 5 and 24 hours. (C) Permeability and (D) cell viability of the same Transwell after 24 hours in response to treatments. Data represented as mean±SD for 3 technical replicates. A two-way ANOVA (Dunett's multiple comparisons test) was done on normalized TEER, all conditions were compared to the pre-treatment conditions. For permeability and cell viability data a one-way ANOVA (Dunett's multiple comparisons test) was used. ****P<0.0001; ***P<0.001; **P<0.01; *P<0.1; ns, not significant.

FIG. 11 illustrates uninflamed UC distal colon derived organoids epithelium monolayers response to proinflammatory cytokine combination IFN-γ/TNF-α/IL-1α (top), IFN-γ/TNF-α (middle) and TNF-α/IL-IL-1α (bottom) induced barrier injury with and without tofacitinib pre-treatment. (A-C) Transepithelial electrical resistance (TEER). (D-F) relative TEER value from the same Transwell before treatment after 5 and 24 hours. (G-I) Permeability and (J-L) cell viability of the same Transwell after 24 hours in response to treatments. Data represented as mean±SD for 3 technical replicates. A two-way ANOVA (Dunett's multiple comparisons test) was done on normalized TEER, all conditions were compared to the pre-treatment conditions. For permeability and cell viability data a one-way ANOVA (Dunett's multiple comparisons test) was used. ****P<0.0001; ***P<0.001; **P<0.01; *P<0.1; ns, not significant.

FIG. 12 illustrates human gastrointestinal tract organoid-derived epithelial monolayers. (A) Human duodenum epithelium monolayer on CNM (top) and after differentiation by eCDM (bottom). (B) Transepithelial electrical resistance (TEER) of human duodenum epithelium monolayers differentiated using different media on day 9. (C) Lucifer yellow (LY) permeability across human duodenum epithelium monolayer, 3 days after differentiation with eCDM. Blank represents LY permeability through Transwell membrane without epithelium monolayer. (D) Pgp1 (ABCB1) and BCRP (ABCG2) gene expression of human duodenum epithelium monolayer on expansion and differentiation, CNM and eCDM respectively. (E) Rhodamine 123 (Rho) transport from basolateral to apical side of human duodenum epithelium monolayer on expansion (EM) and differentiation medium (DM) in presence and absence of Pgp1 inhibitor, PSC833.

FIG. 13 illustrates human gastrointestinal tract organoid-derived epithelial monolayers. Human duodenum and colon epithelium monolayers from expansion CNM (left) and differentiation condition with eCDM (right) stained with H&E, Ki67 and Alcian Blue.

FIG. 14 illustrates polarisation of human gastrointestinal tract organoid-derived epithelial monolayers. (A) TEER of human gastrointestinal tract organoid-derived epithelial monolayers differentiated using eCDM. (B) Lucifer yellow permeability across human gastrointestinal tract organoid-derived epithelial monolayers differentiated using eCDM. “No monolayer” represents a Transwell membrane without epithelium monolayer. TEER=transepithelial electrical resistance; eCDM=enterocyte differentiation medium; Pₐₚₚ=apparent permeability coefficient; Gef=Gefitinib; A=apical application; B=basolateral application; AB=apical and basolateral application.

FIG. 15 illustrates optimization of culture conditions for growth of lung organoid-derived monolayers (lung-A culture). (A) Microscopy images illustrating growth of lung organoid-derived monolayers grown on transwells coated with ECM (Matrigel) and without Matrigel coating. (B) TEER measured for lung organoid-derived monolayers at different cell seeding densities for cultures grown on transwells with and without Matrigel coating.

FIG. 16 illustrates the morphology of lung organoid-derived monolayers grown in different culture media and formats. (A) Images of H&E stained samples from lung-A culture. (B) Images of H&E stained samples from lung-B culture. (C) Images of H&E stained samples from lung-C culture. Ciliated cells are visible on the apical surface of the pseudostratified epithelium layer of cells. LuM=Lung expansion medium; cLuM=Ciliation lung differentiation medium; ALI=air-liquid interface; LLI=liquid-liquid interface.

FIG. 17 illustrates characterisation of the barrier function of lung organoid-derived monolayers grown in different culture media and formats, by measuring TEER. (A), (B), and (C) illustrate TEER values measured for lung-A, lung-B, and lung-C cultures, respectively. LuM=Lung expansion medium; cLuM=Ciliation lung differentiation medium; ALI=air-liquid interface; LLI=liquid-liquid interface. Differentiation was started for cultures grown in cLuM media at the indicated times for each culture, by changing the media from LuM to cLuM media.

FIG. 18 illustrates characterisation of the permeability of lung organoid-derived monolayers to Lucifer Yellow. (A) Schematic diagram of the experimental set-up for Lucifer Yellow permeability assays. Lucifer yellow permeability was measured across lung organoid-derived monolayers grown in different culture conditions. LuM=Lung expansion medium; cLuM=Ciliation lung differentiation medium; ALI=air-liquid interface; LLI=liquid-liquid interface. Lucifer yellow permeability was measured at 4 days or 8 days after differentiation was started for cultures grown in cLuM media, or at corresponding time points for cultures grown in LuM media. (B) illustrates lucifer yellow permeability for lung-A cultures grown in different conditions. (C) illustrates lucifer yellow permeability for lung-B cultures grown in different conditions. (D) illustrates lucifer yellow permeability for lung-C cultures grown in different conditions.

FIG. 19 illustrates characterisation of lung organoid-derived monolayers grown in different culture conditions. LuM=Lung expansion medium; cLuM=Ciliation lung differentiation medium; ALI=air-liquid interface; LLI=liquid-liquid interface. Expression of different genes, which are markers for particular cell types, are shown: KRT5 (lung basal cell marker), SPDEF (goblet cell marker), FOXJ1 (ciliated cell marker), and SFTPA1 (lung alveoli marker). Expression of the transporter proteins OCTN1 and MRP1 was also measured. LuM=Lung expansion medium; cLuM=Ciliation lung differentiation medium; ALI=air-liquid interface; LLI=liquid-liquid interface; Dx+4/8, measurements taken 4 or 8 days after differentiation for cultures grown in cLuM media, or corresponding time points for non-differentiated cultures grown only in LuM media. Gene expression was measured by RT-qPCR for lung-A, lung-B, and lung-C organoid-derived monolayers (FIGS. 19 A, B, E, F, H, and I) and the corresponding lung organoid cultures (FIGS. 19 C, D, G, J, and K). The order of the bars follows the order shown in the figure legend. The order of the bars in FIGS. 19 A, B, E, F, H and I is as follows: Dx+4 LuM LLI, Dx+8 LuM LLI, Dx+4 LuM ALI, Dx+8 LuM ALI, Dx+4 cLuM LLI, Dx+8 cLuM LLI, Dx+4 cLuM ALI, Dx+8 cLuM ALI. The order of the bars in FIGS. 19 C, D, G, J, and K is as follows: 14d LuM, 21d LuM, 28d LuM, 7d LuM+7d cLuM, 7d LuM+14d cLuM, 7d LuM+21d cLuM.

FIG. 20 illustrates characterisation of transport activity of lung organoid-derived monolayers in the ‘accumulation’ assay format. The lung-C culture was used for these experiments. (A) illustrates the timing of cell seeding, initiation of air-liquid interface culture, and the point at which the Calcein AM transport assay was performed. (B) illustrates a schematic diagram of the cell culture format. (C) illustrates a schematic diagram of the ‘accumulation’ assay format. (D) illustrates the fluorescence measurements for accumulated intracellular Calcein AM for lung-C cultures grown in LuM media in LLI and LLI culture formats, in the presence of MK571 (a specific MRP1 inibitor) and PSC833 (a specific P-gp inhibitor). A sample incubated with PBS only (without Calcein AM) was used as a negative control. RFU, relative fluorescence units.

FIG. 21 illustrates characterisation of transport activity of lung organoid-derived monolayers in the ‘pulse-chase’ assay format. The lung-C culture was used for these experiments and the cells were grown as described in FIGS. 20A and B. (A) illustrates a schematic diagram of the ‘pulse-chase’ assay format. (B) illustrates intracellular relative fluorescence values for Calcein AM measured for lung monolayer cultures grown in LuM media in LLI format. (C) illustrates intracellular relative fluorescence values for Calcein AM measured for lung monolayer cultures grown in LuM media in ALI format. A sample incubated in PBS (without Calcein AM) was included as a negative control, along with a blank sample. T=0, fluorescence measured for cultures at 0 minute time point when Calcein AM was removed from the apical and basolateral media. T=2, fluorescence measured for cultures incubated for 2 hours at 37° C. in fresh culture medium without Calcein AM. (D) illustrates the relative fluorescence values for Calcein AM measured in the media in the apical compartment at t=2 for lung organoid-derived monolayers grown in ALI or LLI format. (E) illustrates the relative fluorescence values for Calcein AM measured in the media in the basolateral compartment at t=2 for lung organoid-derived monolayers grown in ALI for LLI format.

FIG. 22 illustrates optimization of culture conditions for growth of kidney organoid-derived monolayers. (A) TEER measured for kidney organoid-derived monolayers at different cell seeding densities for cultures grown on transwells with Matrigel coating. (B) TEER measured for kidney organoid-derived monolayers at different cell seeding densities for cultures grown on transwells with and without Matrigel coating.

FIG. 23 illustrates the morphology of kidney organoid-derived monolayers grown in different culture media. (A) Images of H&E stained samples from kidney-A culture. (B) Images of H&E stained samples from kidney-B culture. (C) Images of H&E stained samples from kidney-C culture. KEM=kidney expansion medium; KDM=kidney differentiation medium; DAC=KEM with 1 μM decitabine added on day 2.

FIG. 24 illustrates characterisation of the barrier function of lung organoid-derived monolayers grown in different culture media, by measuring TEER. (A), (B), and (C) illustrate TEER values measured for kidney-A, kidney-B, and kidney-C cultures, respectively. KEM=kidney expansion medium; KDM=kidney differentiation medium.

FIG. 25 illustrates characterisation of the permeability of lung organoid-derived monolayers to Lucifer Yellow. (A) illustrates lucifer yellow permeability for kidney-A cultures grown in different conditions. (B) illustrates lucifer yellow permeability for kidney-B cultures grown in different conditions. (C) illustrates lucifer yellow permeability for kidney-C cultures grown in different conditions. KEM=kidney expansion medium; D4 KDM=KEM with a change to kidney differentiation medium (KDM) on day 4 after seeding; DAC=KEM with 1 μM decitabine added on day 2.

FIG. 26 illustrates characterisation of gene expression in kidney organoid-derived monolayers grown in different culture conditions. Expression of different genes, which are markers for particular cell types, are shown: ABCC4 (proximal tubule marker), PAX8 (kidney epithelial marker), CLDN10 (loop of Henle marker) and AQP3 (collecting duct marker). Expression of the transporter proteins OCT2, MATE1 and MATE2-K is also shown. 4d/8d KDM: measurements taken after 4 or 8 days of culture in kidney differentiation medium (KDM). Gene expression was measured by RT-qPCR for lung-A, lung-B, and lung-C organoid-derived monolayers (FIGS. 26 A, B, E, G and H) and the corresponding lung organoid cultures (FIGS. 26 C, D, F and I). The order of the bars follows the order shown in the figure legend. The order of the bars in FIGS. 26 A, B, E, G and H is as follows: KEM, DAC, KDM. The order of the bars in FIGS. 26 C, D, F and I is as follows: KEM, 4d KDM, 8d KDM.

FIG. 27 illustrates fluorescence measurements for accumulated intracellular Calcein AM for kidney-C cultures grown in KEM media, with or without addition of 1 uM decitabine (DAC) on day 2 after seeding, in the presence or absence of PSC833 (a specific P-gp inhibitor). A sample incubated with PBS only (without Calcein AM) was used as a negative control. RFU, relative fluorescence units.

FIG. 28 illustrates fluorescence measurements for accumulated intracellular Rhodamine 123 for kidney-C cultures grown in KEM media, with or without addition of 1 uM decitabine (DAC) on day 2 after seeding, in the presence or absence of PSC833 (a specific P-gp inhibitor) and decynium-22 (a specific OCT2 inhibitor). A sample incubated with PBS only (without Calcein AM) was used as a negative control. RFU, relative fluorescence units.

The invention further provides the following numbered embodiments:
1. A method of obtaining an organoid-derived monolayer comprising:
   i. digesting or dissociating one or more organoids into a suspension of single cells and/or organoid fragments;
   ii. seeding a semi-permeable membrane with said suspension; and
   iii. culturing the cells and/or organoid fragments in the presence of an expansion medium until a monolayer is formed.
2. The method of embodiment 1, wherein the method further comprises:
   iv. culturing the monolayer in the presence of a differentiation medium.
3. The method of embodiment 1 or embodiment 2, wherein the monolayer is cultured in the presence of an expansion medium until it reaches transepithelial electrical resistance (TEER) of about 100 Ω·cm².
4. The method of embodiment 2 or embodiment 3, wherein TEER of the monolayer further increases during the step of culturing the monolayer in the presence of a differentiation medium.
5. The method of embodiment 4, wherein TEER of the monolayer reaches more than 500, more than 600, more than 700, more than 800, more than 900, more than 1000, more than 1100, more than 1200, more than 1300, more than 1400 or more than 1500 Ω·cm² during the step of culturing the monolayer in the presence of a differentiation medium.
6. The method of any one of the preceding embodiments, wherein the expansion medium comprises a receptor tyrosine kinase ligand, a BMP inhibitor and a Wnt agonist and, optionally, nicotinamide and a p38 MAPK inhibitor, such as SB202190.
7. The method of any one of embodiments 2-6, wherein the differentiation medium comprises a Notch inhibitor, an EGFR pathway inhibitor and a Wnt agonist.
8. The method of any one of embodiments 2-6, wherein the differentiation medium comprises a Wnt agonist and an inhibitor of Wnt secretion.
9. The method of any one of embodiments 6-8, wherein the receptor tyrosine kinase ligand is a ligand for RTK class I (EGF receptor family) (ErbB family), a ligand for RTK class II (Insulin receptor family), a ligand for RTK class IV (FGF receptor family) or a ligand for RTK class VI (HGF receptor family).
10. The method of embodiment 9, wherein the receptor tyrosine kinase ligand is selected from the group consisting of: epidermal growth factor (EGF), neuregulin, fibroblast growth factor (FGF), hepatocyte growth factor (HGF) and insulin-like growth factor (IGF).
11. The method of any one of embodiments 6-10, wherein the BMP inhibitor is selected from the group consisting of noggin, sclerostin, chordin, CTGF, follistatin, gremlin, tsg, sog, LDN193189 or dorsomorphin.
12. The method of any one of embodiments 6-11, wherein the Wnt agonistis selected from the group consisting of: Rspondin, Wnt conditioned medium and Wnt surrogate.
13. The method of any one of embodiments 7-12, wherein the Notch inhibitor is a gamma secretase inhibitor, optionally selected from the group consisting of: DAPT, dibenzazepine (DBZ), benzodiazepine (BZ) and LY-411575.
14. The method of any one of embodiments 7-13, wherein the EGFR pathway inhibitor is selected from: (1) an EGFR inhibitor, such as Gefitinib, (2) an EGFR and ErbB2 inhibitor, such as Afatinib, (3) an inhibitor of the RAS-RAF-MAPK pathway, (4) an inhibitor of the PI3K/AKT pathway and (5) an inhibitor of the JAK/STAT pathway.
15. The method of embodiment 14, wherein the EGFR pathway inhibitor is an inhibitor of the RAS-RAF-MAPK pathway, e.g. a MEK inhibitor, such as PD0325901.
16. The method of any one of embodiments 8-15, wherein the inhibitor of Wnt secretion is a Porc inhibitor, optionally selected from the group consisting of: IWP 2, LGK974 and IWP 1.
17. The method of any one of the preceding embodiments, wherein:
   i. the monolayer is cultured in the presence of an expansion medium for at least 2 days, at least 3 days, at least 4 days, at least 5 days, at least 6 days, at least 7 days, at least 8 days, at least 9 days or at least 10 days, preferably wherein the monolayer is cultured in the presence of an expansion medium for 3-9 days; and/or
   ii. the monolayer is cultured in the presence of a differentiation medium for at least 3 days, at least 4 days, at least 5 days, at least 6 days, at least 7 days, at least 8 days, at least 9 or more, preferably wherein the monolayer is cultured in the presence of a differentiation medium for 4-8 days.
18. The method of any one of the preceding embodiments, wherein the monolayer is cultured in the presence of an extracellular matrix.
19. An organoid-derived monolayer obtainable or obtained by the method of any one of embodiments 1-18.
20. An organoid-derived monolayer which has transepithelial electrical resistance (TEER) of more than 100 Ω·cm².
21. The organoid-derived monolayer of embodiment 20, wherein the monolayer has TEER of more than 500, more than 600, more than 700, more than 800, more than 900, more than 1000, more than 1100, more than 1200, more than 1300, more than 1400 or more than 1500 Ω·cm².
22. The method or organoid-derived monolayer of any one of the preceding embodiments, wherein the monolayer is derived from the intestine.
23. The method or organoid-derived monolayer of embodiment 22, wherein the monolayer comprises one or more of the following cell types: Lgr5+ stem cell, enterocyte, goblet cell, Paneth cell and enteroendocrine cell.
24. The method or organoid-derived monolayer of any one of the preceding embodiments, wherein the monolayer is derived from a mammal.
25. The method or organoid-derived monolayer of embodiment 24, wherein the monolayer is derived from a human.
26. The method or organoid-derived monolayer of embodiment 25, wherein the human has a disease or disorder of the digestive system, such as inflammatory bowel disease (e.g. Crohn's disease or ulcerative colitis), coeliac disease or leaky gut syndrome.
27. Use of an organoid-derived monolayer according to any one of embodiments 19-26 in an assay assessing epithelial viability, metabolic activity, permeability, barrier function integrity and/or activity of transporter proteins.
28. A method of identifying a compound capable of modulating epithelial viability, metabolic activity, permeability, barrier function integrity and/or activity of transporter proteins comprising:
   i. contacting an organoid-derived monolayer, for example according to any one of embodiments 19-26, with one or more candidate molecules; and
   ii. assessing the viability, metabolic activity, permeability and/or barrier function integrity of the organoid-derived monolayer and/or activity of transporter proteins in the organoid-derived monolayer.
29. A method of assessing the effect of a compound on epithelial viability, metabolic activity, permeability, barrier function integrity and/or activity of transporter proteins comprising:
   i. contacting an organoid-derived monolayer, for example according to any one of embodiments 19-26, with said compound; and
   ii. assessing the viability, metabolic activity, permeability and/or barrier function integrity of the organoid-derived monolayer and/or activity of transporter proteins in the organoid-derived monolayer.
30. The method of embodiment 28 or embodiment 29, wherein the method further comprises contacting the organoid-derived monolayer with one or more proinflammatory cytokines.
31. The method of embodiment 30, wherein the one or more proinflammatory cytokines are selected from the group consisting of: IFN-γ, TNF-α and IL-1α.
32. A method of identifying a mutation associated with epithelial viability, metabolic activity, permeability, barrier function integrity and/or activity of transporter proteins comprising:
   i. assessing the viability, metabolic activity, permeability and/or barrier function integrity of an organoid-derived monolayer and/or activity of transporter proteins in an organoid-derived monolayer, for example an organoid monolayer according to any one of embodiments 19-26; and
   ii. determining the presence of one or more mutations in the genome of one or more cells in the organoid-derived monolayer.
33. A method of diagnosing a disease or affliction that affects epithelial viability, metabolic activity, permeability, barrier function integrity and/or activity of transporter proteins, or determining an increased risk of said disease or affliction, in a human subject comprising:
   i. obtaining an organoid-derived monolayer from said human subject as described in any one of embodiments 1-18; and
   ii. testing the viability, metabolic activity, permeability and/or barrier function integrity of the organoid-derived monolayer and/or activity of transporter proteins in the organoid-derived monolayer,
      wherein a test result above or below a reference value indicates the presence of, or an increased risk of, said disease or affliction in the human subject.
34. The method of embodiment 33, wherein the reference value is a value obtained from a control, e.g. an organoid-derived monolayer obtained from a healthy human subject.
35. The method of embodiment 33 or embodiment 34, wherein the disease or affliction is a disease or disorder of the digestive system, such as inflammatory bowel disease (e.g. Crohn's disease or ulcerative colitis), coeliac disease or leaky gut syndrome.
36. A method of predicting the likelihood of a patient's response to a candidate compound comprising:
   i. obtaining an organoid-derived monolayer from said patient as described in any one of embodiments 1-18;
   ii. contacting the organoid-derived monolayer with said compound; and
   iii. assessing the viability, metabolic activity, permeability and/or barrier function integrity of the organoid-derived monolayer and/or activity of transporter proteins in the organoid-derived monolayer.
37. The use or method of any one of embodiments 27-36, wherein assessing the barrier function integrity of the organoid-derived monolayer comprises measuring TEER of the organoid-derived monolayer.
38. The use or method of any one of embodiments 27-37, wherein assessing the permeability of the organoid-derived monolayer comprises measuring the rate of passive diffusion of a reporter compound across the monolayer.
39. The use or method of embodiment 38, wherein said reporter compound is a dye, optionally a fluorescent dye, such as Lucifer yellow.
40. The use or method of any one of embodiments 27-39, wherein assessing the activity of transporter proteins comprises measuring the rate of transport of a substrate of a transporter protein across the monolayer, optionally in the presence of an inhibitor of said transporter protein.
41. The use or method of embodiment 40, wherein the substrate is a dye, such as Rhodamine 123.

### EXAMPLES

### Example 1. Preparation of Epithelial Monolayers from Human Normal Intestinal Organoids

Although the epithelial monolayers in this protocol are prepared from human normal intestinal organoids, the protocol can be applied and optimized for other organoid models. Epithelial organoid monolayers are cultured in intestinal organoid expansion medium containing Wnt to support stem cell proliferation and represent intestinal crypt cellular composition. Intestinal organoids can be enriched to have different intestinal epithelial fates, such as enterocytes, Paneth, goblet, and enteroendocrine cells, by modulating Wnt, Notch, and epidermal growth factor (EGF) pathways. Here, after the establishment of monolayers in expansion medium, they are driven toward more differentiated intestinal epithelial cells, as described previously (van Es, J. H. et al. Wnt signalling induces maturation of Paneth cells in intestinal crypts. Nature Cell Biology. 7(4), 381-386 (2005); van Es, J. H. et al. Dlll marks early secretory progenitors in gut crypts that can revert to stem cells upon tissue damage. Nature Cell Biology. 14 (10), 1099-1104 (2012).; de Lau, W. B. M., Snel, B., Clevers, H. C. The R-spondin protein family. Genome Biology. 13 (3), 1-10 (2012); Basak, O., Beumer, J., Wiebrands, K., Seno, H., van Oudenaarden, A., Clevers, H. Induced quiescence of Lgr5+ stem cells in intestinal organoids enables differentiation of hormone-producing enteroendocrine cells. Cell Stem Cell. 20 (2), 177-190.e4 (2017); Beumer, J. et al. Enteroendocrine cells switch hormone expression along the crypt-to-villus BMP signalling gradient. Nature Cell Biology. 20 (8), 909-916 (2018); Yin, X., Farin, H. F., van Es, J. H., Clevers, H., Langer, R., Karp, J. M. Niche-independent high-purity cultures of Lgr5+ intestinal stem cells and their progeny. Nature Methods. 11 (1), 10⁶-112 (2014)). For screening purposes, depending on the mode of action of the compound of interest, its target cells, and the experimental conditions, the monolayers can be driven toward the cellular composition of choice to measure the effects of the compound with relevant functional readouts.

### 1. Preparing Reagents for Culture

NOTE: Perform all steps inside a biosafety cabinet and follow standard guidelines for working with cell cultures. Ultraviolet light is used for 10 min before starting up the biosafety cabinet. Before and after use, the surface of the biosafety cabinet is cleaned with a tissue paper drenched in 70% ethanol. To facilitate the formation of three-dimensional drops of extracellular matrix (ECM), keep a prewarmed stock of 96-, 24-, and 6-well plates ready in the incubator at 37° C.
1. Basal medium preparation
   1. Prepare basal medium (BM) in a 500 mL of Advanced Dulbecco's Modified Eagle Medium with Ham's Nutrient Mixture F-12 (Ad-DF) medium bottle by adding 5 mL of 200 mM glutamine, 5 mL of 1 M 4-(2-hydroxyethil)-lpiperazineethanesulfonic acid (HEPES), and 5 mL of penicillin/streptomycin (pen/strep) solutions (10,000 U/mL or 10,000 μg/mL). Store it in the refrigerator at 4° C. for at least 4 weeks.
2. Wnt sources
   1. Prepare Wnt3a-conditioned medium (Wnt3aCM) according to the previously described method (Boj, S. F. et al. Forskolin-induced swelling in intestinal organoids: An in vitro assay for assessing drug response in cystic fibrosis patients. Journal of Visualized Experiments. 2017 (120), 1-12 (2017)). NOTE: Recently, a next-generation surrogate Wnt (NGS-Wnt), which also supports expansion of human intestinal organoids, has been generated (Miao, Y. et al. Next-generation surrogate Wnts support organoid growth and deconvolute Frizzled pleiotropy in vivo. Cell Stem Cell. 27 (5), 840-851 (2020)).
3. Intestinal organoid base medium preparation
   NOTE: Where possible, use growth factors and reagents according to the manufacturer's recommendations. Where possible, use small aliquots and avoid freeze-thaw cycles; functional growth factors are advantageous for successful organoid culture.
   1. Prepare concentrated 2× intestinal organoid base medium (2×IBM) by supplementing BM with 1 μM A83-01, 2.5 mM N-acetylcysteine, 2×B27 supplement, 100 ng/mL human epidermal growth factor (hEGF), 10 nM gastrin, 200 ng/mL hNoggin, and 100 μg/mL of an antimicrobial formulation for primary cells.
   2. Aliquot the 2×IBM and freeze at −20° C. for up to 4 months. When needed, thaw an aliquot overnight at 4° C. or for several hours at room temperature (RT).
   3. To prepare intestinal organoid expansion medium (IEM, also referred to herein as CNM), supplement 2×IBM with either 50% Wnt3aCM or 50% BM and 0.5 nM NGS-Wnt, 250 ng/mL human Rspondin-3 (hRspo3), 10 mM nicotinamide, and 10 μM SB202190.
4. Intestinal Organoid Differentiation Medium Preparation
   1. Prepare enterocyte differentiation medium (eDM) by supplementing 2×IBM with 50% BM, 250 ng/mL hRspo3, and 1.5 μM Wnt pathway inhibitor (IWP-2). Store eDM at 4° C. for up to 10 days.
   2. Prepare combination differentiation medium (cDM) by supplementing 2×IBM with either 40% BM and 10% Wnt3aCM or 50% BM and 0.1 nM NGS-Wnt, 250 ng/mL hRspo3, 10 μM DAPT and 100 nM PD0325901. Store cDM at 4° C. for up to 10 days.
5. Manipulation of extracellular matrix (ECM)
   NOTE: Prepare the extracellular matrix (ECM) according to the manufacturer's recommendation.
6. Thaw ECM overnight on ice; transfer the ECM from the bottle to a 15 mL conical tube using a 5 mL pipette, both pre-cooled at −20° C. Refreeze aliquots only once at −20° C. Once thawed, store the ECM in a refrigerator at 4° C. for up to 7 days. Incubate for at least 30 min on ice before use.
7. NOTE: It is advantageous to mix ECM properly and ensure that it is cold before embedding crypts or organoids.

### 2. Organoid Cultures

1. Passaging of intestinal organoids for epithelial monolayer preparation
   1. Passage organoids 3 days prior to harvest to prepare the monolayers. Resuspend the organoids in 1-1.5× the starting volume of IEM/ECM to have a higher density and expansion potential when they are harvested for monolayer preparation.

### 3. Epithelial Monolayer Preparation

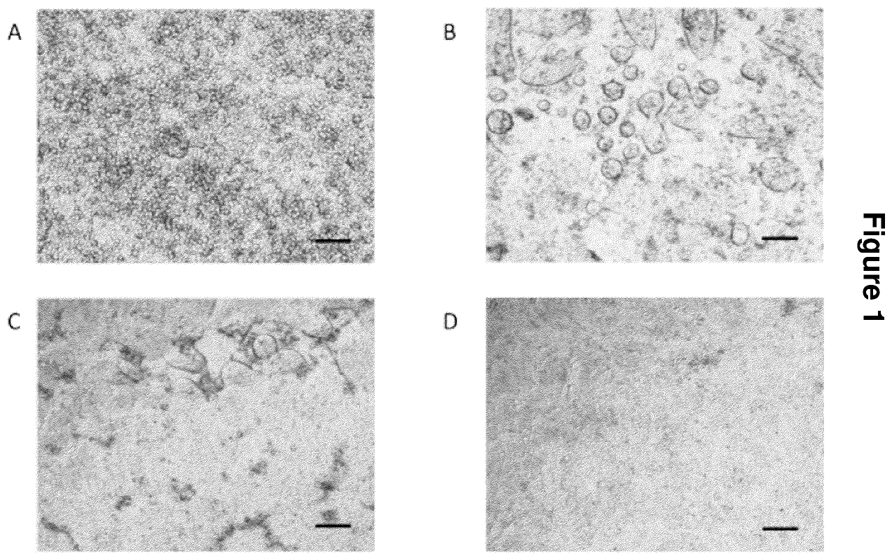

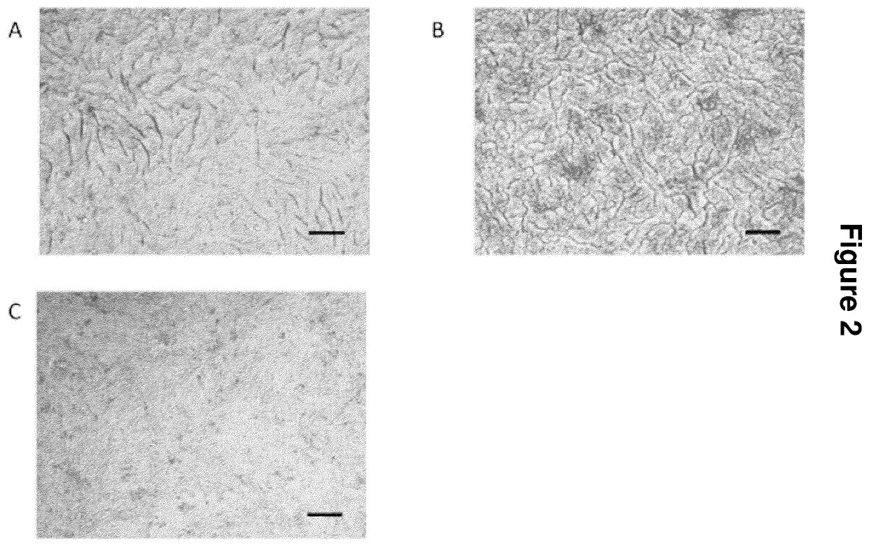

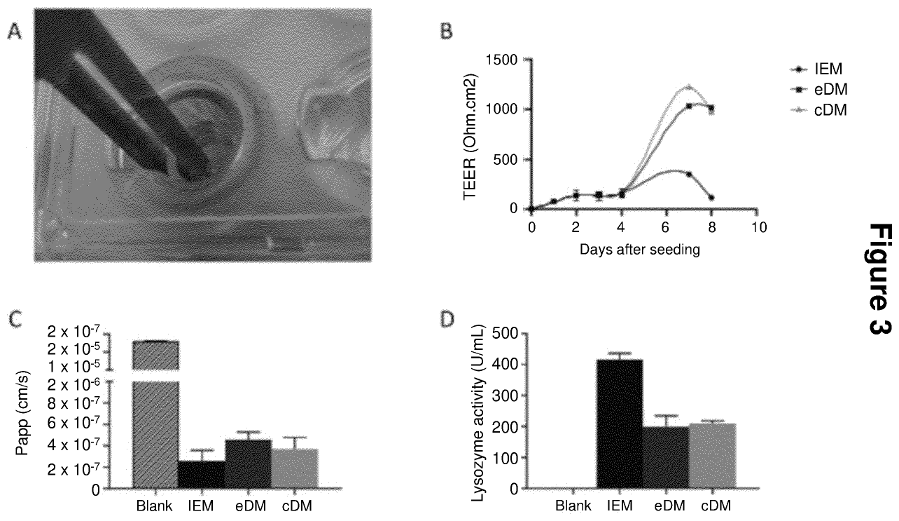

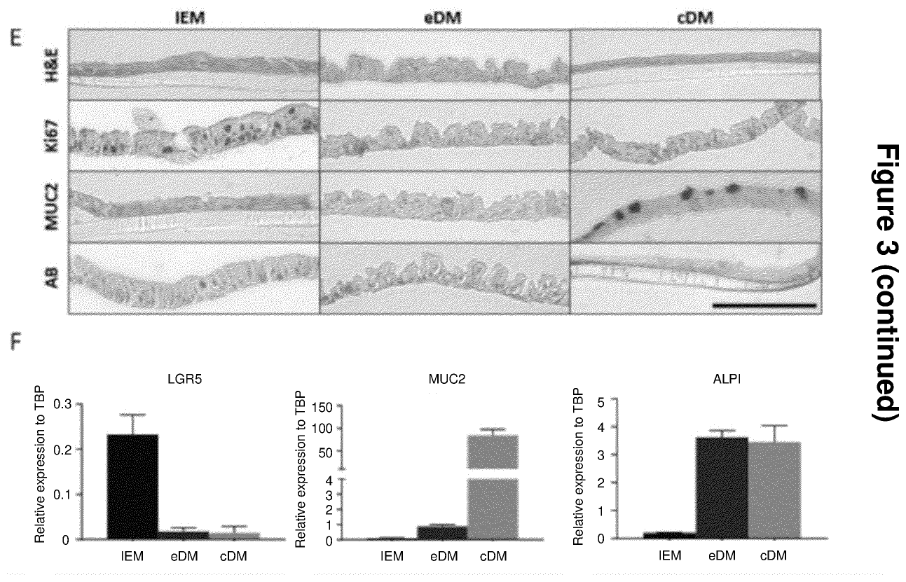

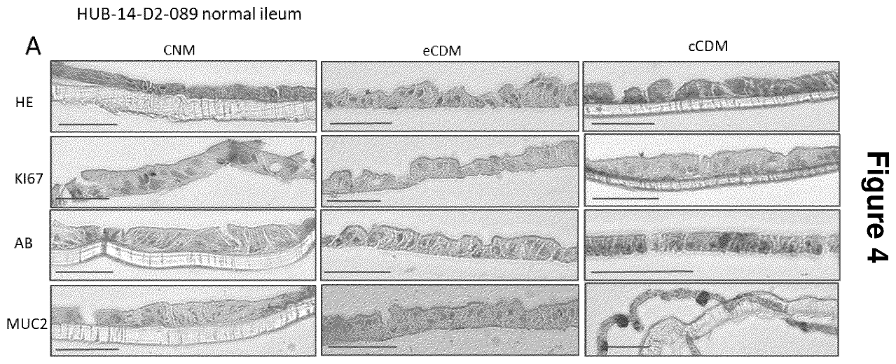

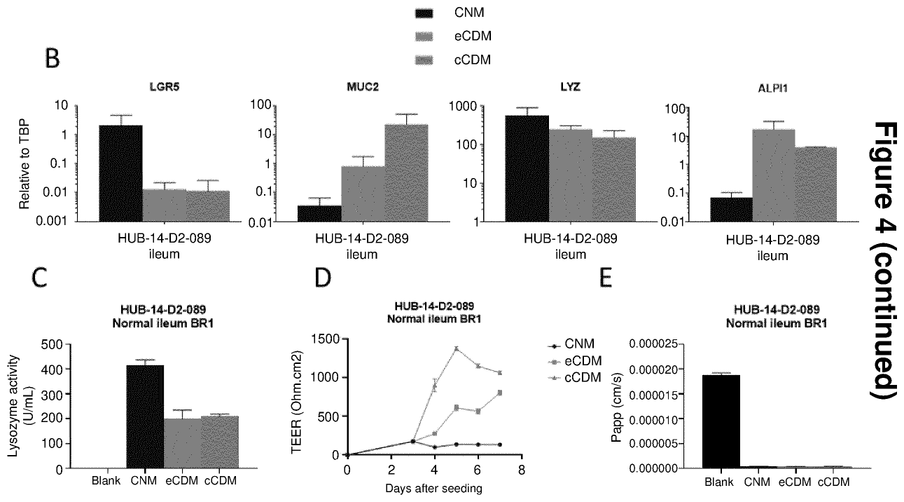

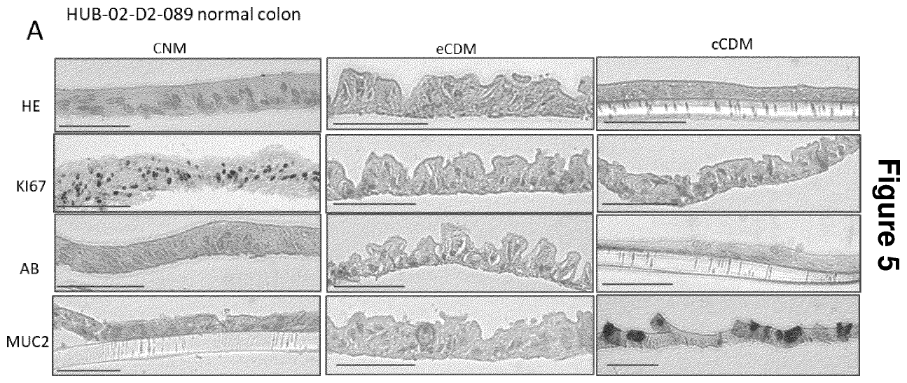

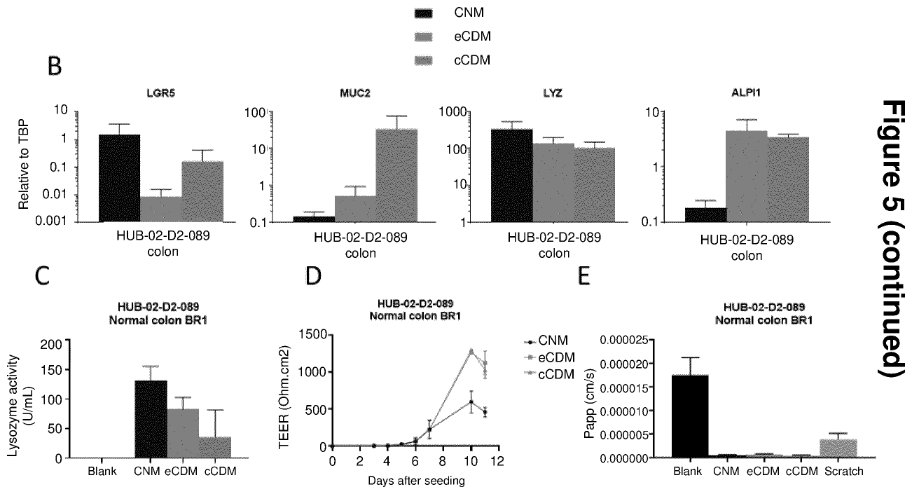

1. Culture epithelial monolayers on both 24-well and 96-well membrane inserts with a variety of available plate types. Use high-throughput system (HTS) membrane inserts for both sizes as these contain an integral tray with the membrane inserts and a receiver plate. For the 24-well format, use plates with separate removable membrane inserts.
   NOTE: Different membrane types (polyethylene terephthalate (PET) or polycarbonate) and pore sizes (0.4-8.0 μm) are available and can be used depending on experimental needs. Monolayers can only be imaged by brightfield when inserts with PET membranes are used. Light-tight membranes block fluorescent light leakage from the apical to the basolateral compartment and can be considered when dynamic transport or permeability of fluorescently labeled substrates is studied. The current protocol uses 24-well membrane inserts; adaptations for 96-well membrane inserts are available. Depending on the density, morphology, and size of the organoids, 6 wells of a 6-well plate are enough for seeding a full 24-well plate of membrane inserts.
2. Coating membrane inserts with ECM
   NOTE: If there are doubts about having enough cells, coat the inserts after 10 counting the cells. This is to prevent unnecessary coating and loss of the expensive membrane inserts.
   1. Place the membrane inserts into the support plate in the biosafety cabinet. Dilute the ECM 40× in ice-cold Dulbecco's phosphate-buffered saline (DPBS) with Ca2+ and Mg2+, and pipet 150 μL of the diluted ECM into the apical compartment of each insert. Incubate the plate at 37° C. for at least 1 h.
3. Preparation of cells for seeding
   1. Prewarm aliquots of the cell dissociation reagent in the water bath (37° C.). Prepare 2 mL of the reagent for each well of a 6-well plate.
   2. Transfer the culture plate containing the organoids from the incubator to the biosafety cabinet. Process and passage the organoids, as previously described. Do not pool multiple tubes into one tube.
   3. Fill the tube, containing organoids from a maximum of 3 wells of a 6-well plate, up to 12 mL with DPBS (without Ca²⁺ and Mg²⁺), and pipet up and down 10× using a 10 mL pipette. Centrifuge at 85×g for 5 min at 8° C., and aspirate the supernatant without disturbing the organoid pellet.
   4. Add 2 mL of the prewarmed cell dissociation reagent per well of a 6-well plate used as the starting material and resuspend. Incubate the tubes diagonally or horizontally for 5 min in the water bath at 37° C., to prevent the sinking of the organoids to the bottom of the tube.
   5. Pipet up and down 10× using a 5 mL sterile plastic pipette or a P1000 pipette, depending on the total volume of the cell dissociation reagent. Check the organoid suspension under the microscope to see if a mixture of single cells and some cell clumps consisting of 2-4 cells has formed (FIG. 3B). If needed, continue the digestion by repeating steps 3.3.4-3.3.5 until single cells and small clumps of cells are visible in the mixture. NOTE: Where possible avoid digesting the organoids fully to single cells. It is advantageous to have some small groups of cells (i.e., groups of 2-4 cells).
   6. Stop cell dissociation by adding up to 12 mL of BM to the cell suspension. Centrifuge at 450×g for 5 min at 8° C., and aspirate the supernatant without disturbing the cell pellet. When handling the same organoid culture in several 15 mL conical tubes, pool the cell pellets and resuspend them in 12 mL of BM.
   7. Filter the cell suspension through a 40 m strainer prewetted with BM, and harvest the flow-through into a 50 mL conical tube. Wash the strainer with 10 mL of BM, and harvest the flow-through into the same 50 mL conical tube.
   8. Transfer the strained cell suspension into two new 15 mL conical tubes. Centrifuge at 450×g for 5 min at 8° C., and aspirate the supernatant without disturbing the cell pellet. Resuspend the cells in 4 mL of IEM supplemented with 10 μM ROCK inhibitor per full culture plate used as starting material.
   9. Mix a small amount of cell suspension in a 1:1 ratio with trypan blue for counting. Count the live, not blue, cells, and calculate the total number of live cells. In small clumps, count each individual cell.
   10. Prepare a cell suspension containing 3×10⁶ live cells per mL of IEM supplemented with 10 μM ROCK inhibitor.
4. Seeding cells on polyester membrane inserts
   1. Carefully aspirate DPBS from the ECM-coated inserts (step 3.2.1), whilst keeping the plate horizontally. Pipet 800 μL of IEM supplemented with ROCK inhibitor into each basolateral compartment. Pipet 150 μL of the cell suspension prepared in step 3.3.10 onto the ECM-coated membrane in the apical compartment dropwise. Per plate, be sure to have at least one “blank” well with BM only.
   2. Once the cells have sedimented onto the membrane, measure transepithelial electrical resistance (TEER), as described herein, and image the membrane inserts using a microscope. Place the plate in the incubator at 37° C. and 5% CO2. Measure TEER every day, and acquire images regularly to monitor monolayer formation (FIG. 1A-D).
5. Refreshing monolayers
   NOTE: It is advantageous to refresh the medium every 2-3 days, adhering to the following order to maintain a positive hydrostatic pressure above the cells and prevent cells from being pushed off the membrane. While refreshing the medium, take care that the monolayer, which is visible upon aspiration of the medium, is not damaged by the pipette tip.
   1. Remove the medium from the basolateral compartments of the plate containing the membrane inserts. Then, carefully aspirate the medium from the apical compartments of the membrane inserts.
   2. Add 150 μL of fresh IEM dropwise to each apical compartment, and then add 800 μL of fresh IEM to each basolateral compartment.
6. Enrichment of the monolayer for desired intestinal epithelial cell types
   1. Allow the monolayer to become confluent in IEM, corresponding to a TEER value of around 100 Ω·cm². Check under the microscope to determine whether the monolayers have completely formed (FIG. 1D) and for the absence of holes (as seen in FIG. 1B,C).
   2. Carefully remove IEM from the basolateral and apical compartments of the membrane inserts, and replace with either eDM or cDM as prepared in section 1.4. Culture the monolayer for another 3-4 days in the specific differentiation medium to get the organoid cells enriched with the desired specific cell type. Refresh the medium every 2-3 days, as described in section 3.4.
   3. Measure TEER daily, and acquire images regularly if desired (FIG. 2A-C).
      NOTE: The TEER value that indicates a fully organized enriched monolayer varies per organoid culture; typically TEER values increase to 600 and can increase up to 1000 Ω·cm² after 3 days in differentiation media and are stable for 3-5 days.

### 4. Representative Results

When passaging organoids for the preparation of monolayers, be sure to plate them at a high density to ensure sufficient cell numbers for seeding the monolayers, and let them grow for three days so they are in optimal expansion conditions. Organoids can be harvested for monolayer preparation at appropriate size and density, where 6 wells of a 6-well plate, each containing 200 μL of organoid domes, are typically enough for seeding a full 24-well plate of membrane inserts. After the preparation of a single-cell suspension with the cell dissociation reagent, single cells and small clumps of cells should be visible, and live cells can be counted. Dead cells stained with trypan blue should be excluded from counting. The single cells and small clumps are then seeded in the membrane inserts as seen in FIG. 1A. Monolayer formation is visible after 1-3 days (FIG. 1B,C), and the monolayers will be generally be confluent after 3-6 days depending on the organoid culture (FIG. 1D). Monolayers stay in expansion medium until they are confluent, after which they can be enriched with, amongst others, enterocytes or goblet cells using different enrichment media. FIG. 2A shows a monolayer that was cultured for 8 days in expansion medium (IEM). When enriched with enterocytes (eDM), a structure is seen, as in FIG. 2B, while monolayers exposed to combination medium (cDM) show a smoother structure (FIG. 2C).

Monolayer formation can be quantitatively followed by measuring TEER (FIG. 3A). A completely confluent monolayer has a TEER value of ˜100 Ω·cm², which increases to ˜1000 Ω·cm² when exposed to either differentiation medium (FIG. 3B). Monolayers in all medium conditions are impermeable to Lucifer Yellow (0.45 kDa), while an increase in apparent permeability (Papp) can be seen when the monolayers were purposely scratched (FIG. 3C). Lysozyme secretion by ileal monolayers cultured in IEM was higher than that of monolayers cultured in IEM until confluent and for another 4 days in eDM or cDM (denoted as +subsequent eDM or cDM) (FIG. 3D). Monolayers cultured in IEM, IEM+subsequent eDM or IEM+subsequent cDM show different morphology, as can be observed with H&E staining (FIG. 3E). While colon organoid-derived epithelial monolayers in IEM and cDM media have a smooth apical surface, enterocyte-differentiated monolayers present an invaginated apical morphology in the absence of Wnt. Ki67-positive proliferative cells can be detected in expansion conditions only. Alcian Blue and MUC2 stain mucus produced by goblet cells, which is visualized in the monolayers differentiated in eDM and more prominently in cDM when Wnt, Notch, and EGF signaling are inhibited, respectively (FIG. 3E). Upon differentiation, proliferative cells decrease while goblet cell and enterocyte marker gene expression increases in comparison to that observed under IEM conditions, as shown by LGR5, MUC2, and ALPI gene expression quantification by qRT-PCR, respectively (FIG. 3F).

A protocol essentially as described above was also shown to be successful for generating monolayers from dog and rat intestinal organoids. The rat organoid-derived monolayers had TEER of about 20 Ω·cm², whilst the dog organoid-derived monolayers reached TEER of more than 1000 Ω·cm².

### Example 2. Human GI Tract Epithelium Monolayer Establishment, Differentiation and Characterization

Currently, intestinal permeability and testing the effect of compounds on barrier function is either studied by transformed cell lines, such as the colonic adenocarcinoma cell line Caco-2, T84 or HT-29, or primary epithelial GI tract tissue mounted on Ussing chambers. Although cell lines can form differentiated and polarized monolayers, containing intestinal enterocyte- and Goblet-like cells, many different enzymes and transporters are aberrantly expressed in these cell lines, therefore having a reduced complexity and physiological relevance. In addition, since cell lines are driven from a single donor, they do not represent patient population heterogeneity. Epithelium monolayer preparations from intestinal organoids would combine cell line expandability with the high physiological and patient relevance of primary tissue. Thus, we sought the establishment of monolayers using human ileum and colon organoids. For this purpose, organoids were digested into single cells and seeded on transwell membranes in CNM, eCDM and cCDM culture conditions.

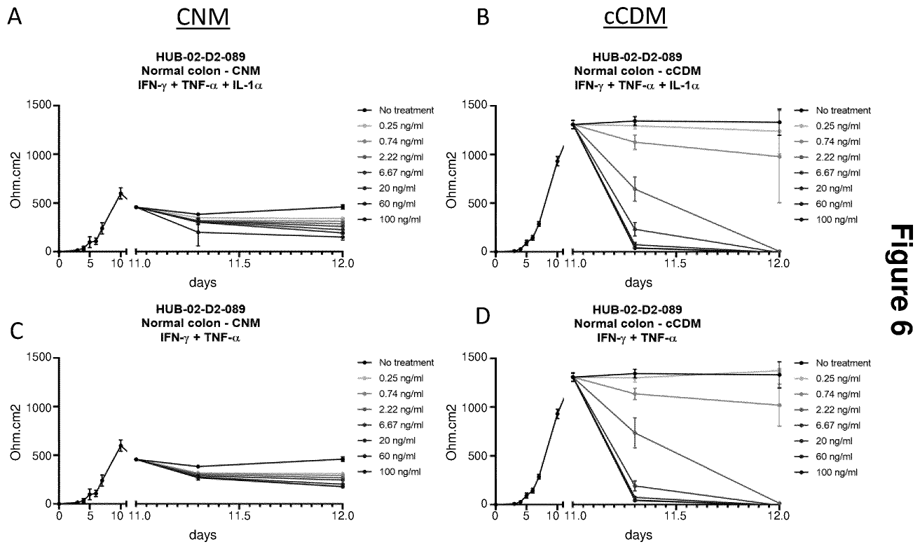

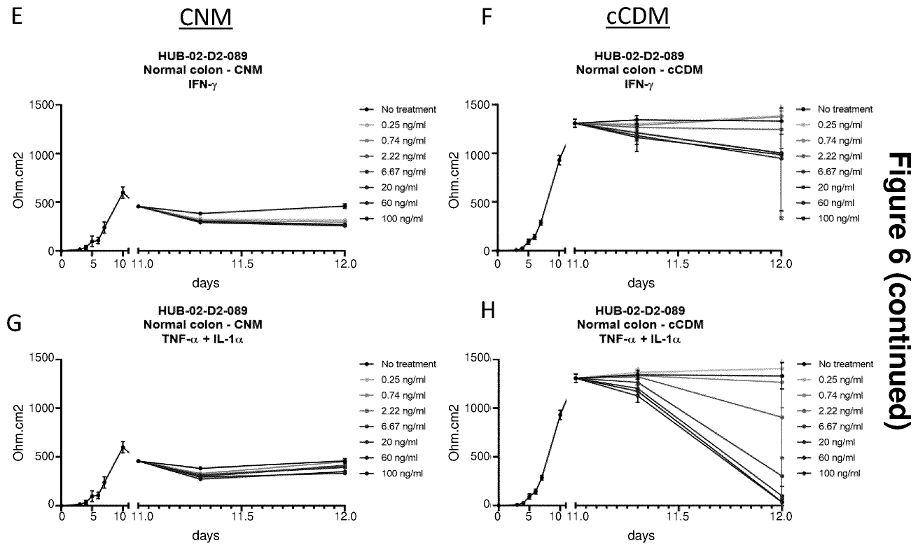

In CNM conditions, H&E stain of epithelium monolayer cross sections showed simple squamous epithelium for both ileum (FIG. 4A) and colon (FIG. 5A) models. Further histological stains showed the presence of proliferative cells (KI67) and the absence of Goblet cells (Alcian blue and MUC2) (FIGS. 4A and 5A, CNM condition). Gene expression analysis using RT-qPCR of LGR5 and MUC2 genes confirmed histological observation, and lack of ALPI1 expression indicated the absence of enterocytes (FIGS. 4B and 5B) in monolayers generated in CNM conditions. Lysozyme (LYZ) expression was detected in both ileum and colon organoid-derived monolayers (FIGS. 4B and 5B), and activity measured in supernatants collected from the apical chambers of the transwells (FIGS. 4C and 5C).

Ileum- and colon-derived monolayers cultured in eCDM condition changed their morphology to a simple columnar epithelium and showed less proliferative (KI67+) and LGR5+ stem cells (FIGS. 4A, 4B, 5A and 5B, eCDM condition). Alcian blue and MUC2 stains revealed no or a limited number of Goblet cells in ileum and colon epithelium monolayers, respectively. This was confirmed by RT-qPCR, which also showed lower levels of MUC2 expression in eCDM culture condition compared to cCDM (FIGS. 4A, 4B, 5A and 5B). In contrast, expression analysis of ALPI1 suggested strong enrichment of the ileum-derived monolayer with enterocytes in the eCDM culture condition (FIG. 4B). Lastly, LYZ mRNA levels and lysozyme activity were reduced in eCDM compared to CNM culture conditions, as expected due to WNT pathway inhibition in eCDM culture condition (FIGS. 4B, 4C, 5B and 5C).

In cCDM culture conditions, similar to eCDM, no proliferative cells or stem cells were observed and LYZ1 expression was reduced (FIGS. 4B, 4C, 5B and 5C, cCDM condition). ALPI1 mRNA expression furthermore indicated that enterocyte differentiation was reduced compared to eCDM. However, Goblet cells appeared at higher numbers in both ileum- and colon-derived epithelium monolayers, as revealed by both Alcian Blue and MUC2 staining and RT-qPCR expression analysis (FIGS. 4B, 4C, 5B and 5C).

Epithelium monolayer formation and integrity was evaluated by Trans Epithelial Electrical Resistance (TEER) which reached between 100 to 200 Ω·cm² on day 3-7, in CNM culture condition. After reaching a TEER of at least 100 Ω·cm², monolayers were differentiated, and their differentiation was followed by TEER for four additional days. Among the tested culture conditions, CNM maintained a stable TEER, whereas eCDM and cCDM increased TEER to ˜1000 Ω·cm², indicating an increased barrier integrity (FIGS. 4 and 4 D), possibly caused by the increased expression of tight junction proteins. Indeed, RT-qPCR analysis shows that the expression of tight junction protein complex changes such as ZO-1 and OCLN, in ileum derived monolayers in a 24-well format.

Next to TEER measurements (FIGS. 4D and 5D), paracellular permeability of monolayers after 4 days in differentiation medium was evaluated by passive diffusion of Lucifer Yellow (LY) from the apical to basolateral side. Damaging the monolayers by making a scratch, led to diffusion of LY to the basolateral side. However, the level of LY was not equal to blank wells (no monolayer) suggesting that LY was sticking to the monolayers. In both ileum- and colon-derived monolayers, no LY diffusion from the apical to the basolateral compartment was observed, suggesting that both differentiated and undifferentiated epithelium monolayers were impermeable (FIGS. 4E and 5 E).

Epithelium monolayer formation and differentiation experiments were carried out in at least two biological replicates to evaluate assay reproducibility. Representative histological sections stained with KI67, AB (Alcian blue) and MUC2 are shown in FIGS. 4A and 5A. Gene expression was analysed by RT-qPCR and results are presented as the average of at least two biological replicates in FIGS. 4B and 5B. Individual TEER, permeability and lysozyme activity measurements are presented in FIG. 4C, 4D, 5C 5D. Since TEER measurement of the first colon biological replicate in CNM condition (FIG. 5D, middle panel) was not reproduced in the second biological replicate, we performed a third biological replicate, which results were comparable with the first replicate. Spontaneous differentiation or growth factor depletion such as Wnt in the CNM culture condition in the second replicate could explain the observed result in second replicate.

Apart from this later observation, the results from biological replicates were comparable, indicating organoids can be used to establish human epithelial monolayers from different GI tract regions. These epithelium monolayers were polarized and could be differentiated to enterocytes and mucus producing Goblet cells, while their barrier integrity increased and remained impermeable to LY.

### Example 3. Development of an In Vitro Biological System to Mimic Components of IBD Pathophysiology, with Robust Readouts for Barrier Function Pathways

A screening platform based on organoid-derived epithelium monolayers was developed, optimized and validated herein to be used as a robust, functional read out for barrier function.

### Organoid-Derived Epithelium Monolayer

Despite comparable TEER values between eCDM and cCDM conditions (FIG. 4D, 5D), the higher mucus production in intestinal organoids cultured in cCDM, led the selection of CNM and cCDM culture conditions for further assay development for barrier function integrity (FIGS. 4A, 4B, 5A and 5B).

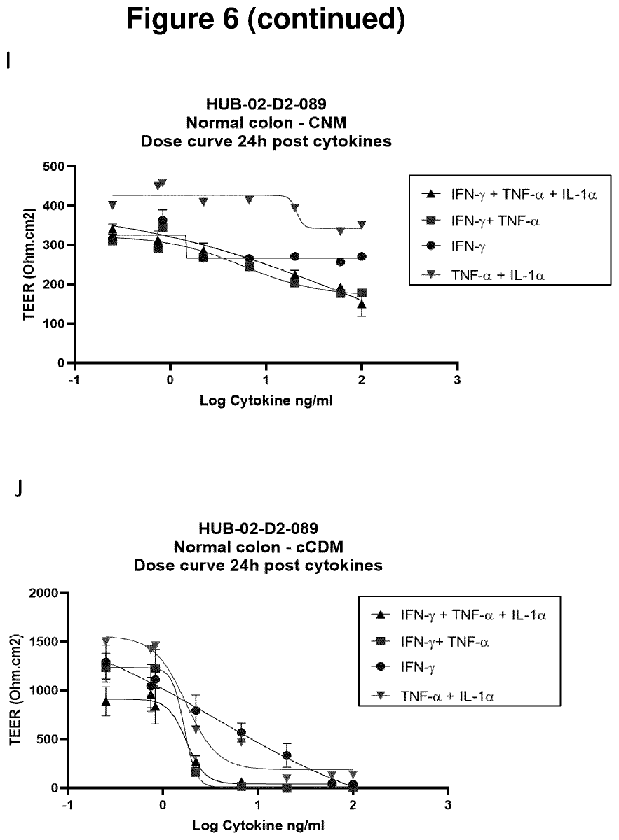

In order to explore the effect of several proinflammatory cytokines in the barrier function of monolayers generated from colon-derived organoids, the most relevant proinflammatory cytokines implicated in IBD (IFN-γ, TNF-α and IL-1α) were titrated to obtain EC₅₀ values for these cytokines within a 24 h assay window (FIG. 6A-H). As IFN-γ has major impact in inducing barrier damage via JAK-STAT signalling pathway, we titrated different combinations of three indicated proinflammatory cytokines to resolve TNF-α and IL-1α synergy in combination with IFN-γ. Titrations of these three cytokines, from 0.25 ng/ml up to 100 ng/ml, resulted in a dose dependent decrease of TEER in both culture conditions after 5 hours. This effect remained relatively stable for up to 24 hours in CNM (FIG. 6A), but further TEER loss was observed in cCDM, particularly for concentrations higher than 2 ng/ml, suggesting a complete loss of barrier integrity, possibly caused by cell death (FIG. 6B). The lower sensitivity of organoid-derived epithelium monolayers cultured in CNM correlated with a lower expression of IFN-γ receptor (IFNGR1) compared to the cCDM culture condition.

The presence of two further cytokines in combination with IFN-γ made epithelial monolayers more vulnerable to proinflammatory cytokine damage as it appeared in triple combination of IFN-γ, TNF-α and IL-1α (EC₅₀ 1.77) and double combinations of IFN-γ/TNF-α (EC₅₀ 1.67) as compared with single treatments with IFN-γ (EC₅₀ 3.71) (FIG. 6J, Table 5). TNF-α/IL-1α combination had comparable effect (EC₅₀ 1.74) with IFN-γ combinations after 24 hours, but not after 5 hours (FIGS. 6A-H and J, Table 5). Since cCDM culture condition represented more physiologically relevant cellular heterogeneity, the triple combination of proinflammatory cytokines showed a strong IFN-γ-specific effect on barrier integrity, and had an increased dynamic range, providing extended signal window for screening purposes, cCDM culture condition was chosen for further assay development. The EC₅₀ of triple-combined, proinflammatory cytokines on colon organoid-derived epithelium monolayers cultured in cCDM was determined as 2 ng/ml (FIGS. 6 I and J) and concentrations ranging around this point were used in the follow up experiments.

TABLE 5
EC₅₀ calculated from EC₅₀ dose response curves

| Cytokine combination | EC₅₀ |
|---|---|
| IFN-γ/TNF-α/IL-1α | 1.77 |
| IFN-γ/TNF-α | 1.67 |
| TNF-α/IL-1α | 1.74 |
| IFN-γ | 3.71 |

Tofacitinib Protects Epithelium Monolayers from Proinflammatory Cytokine-Induced Barrier Injury

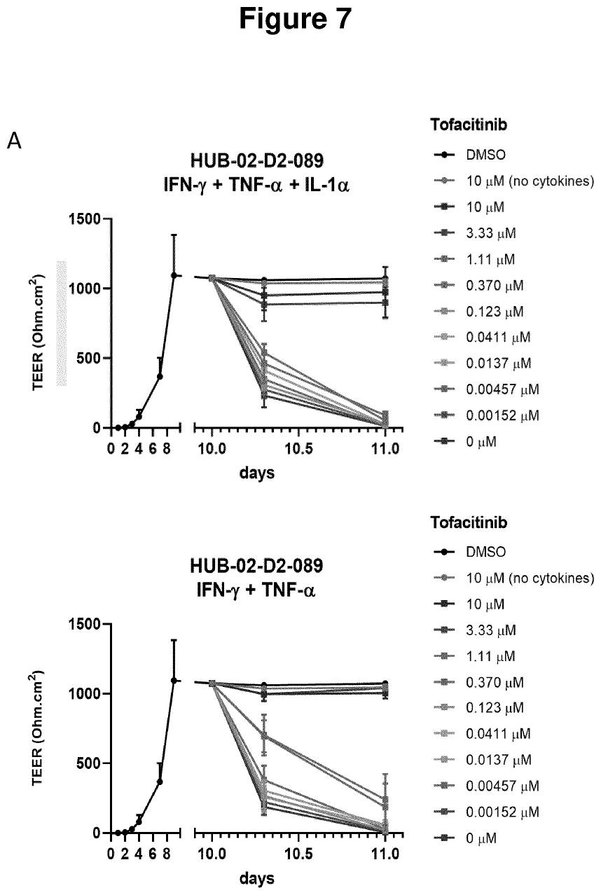

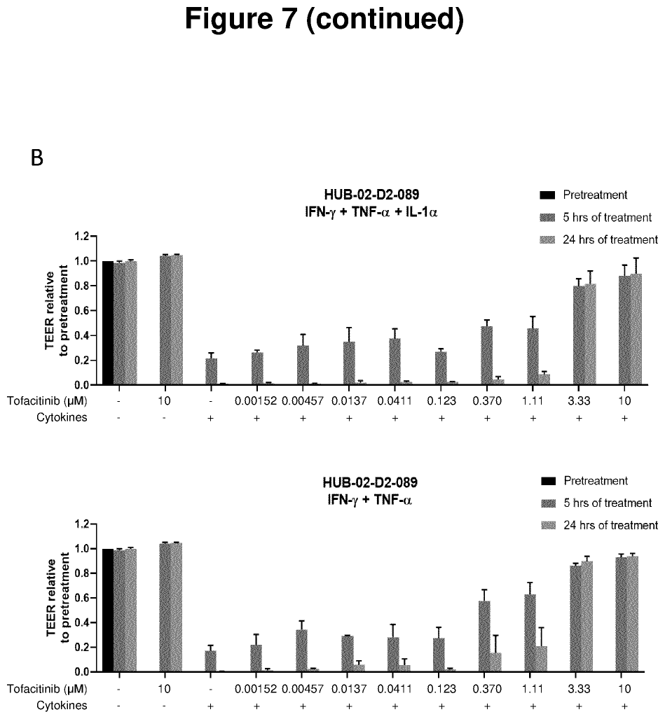

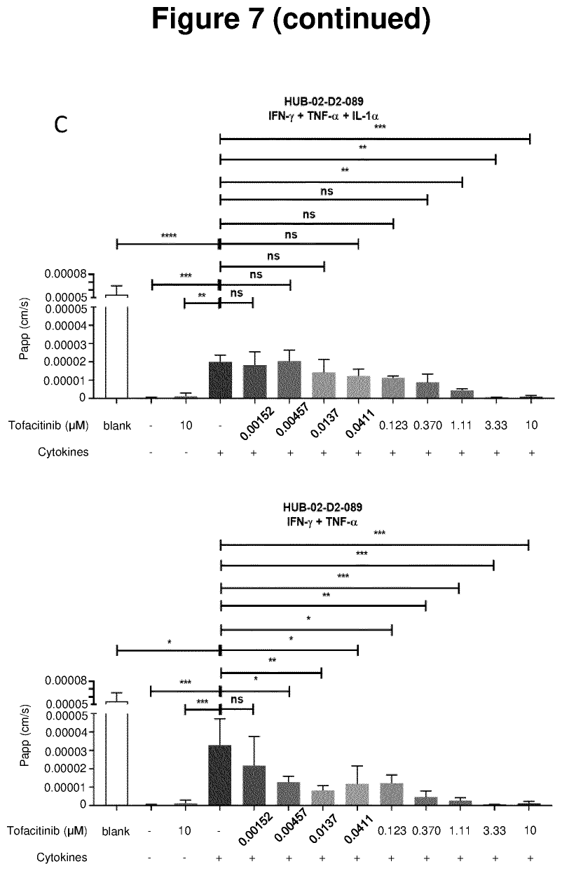

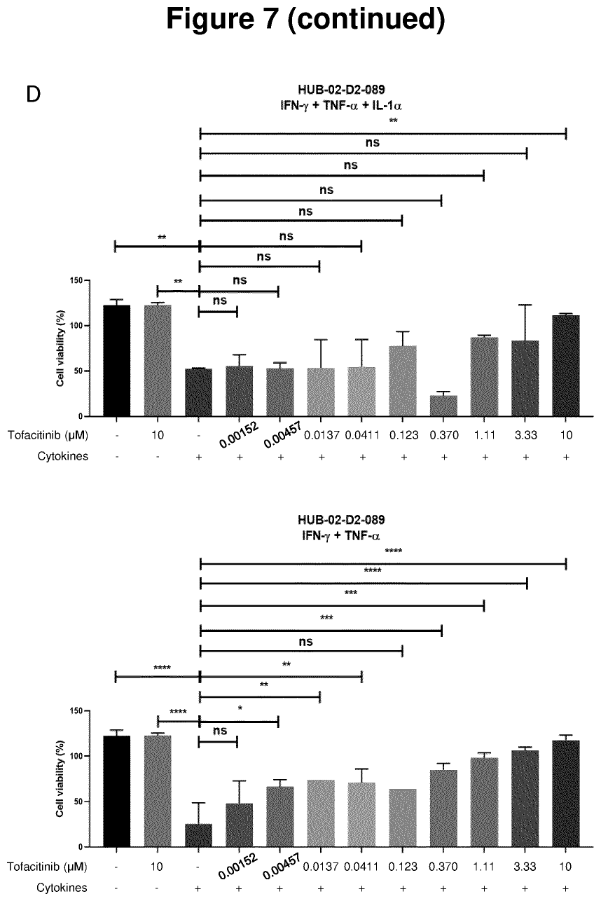

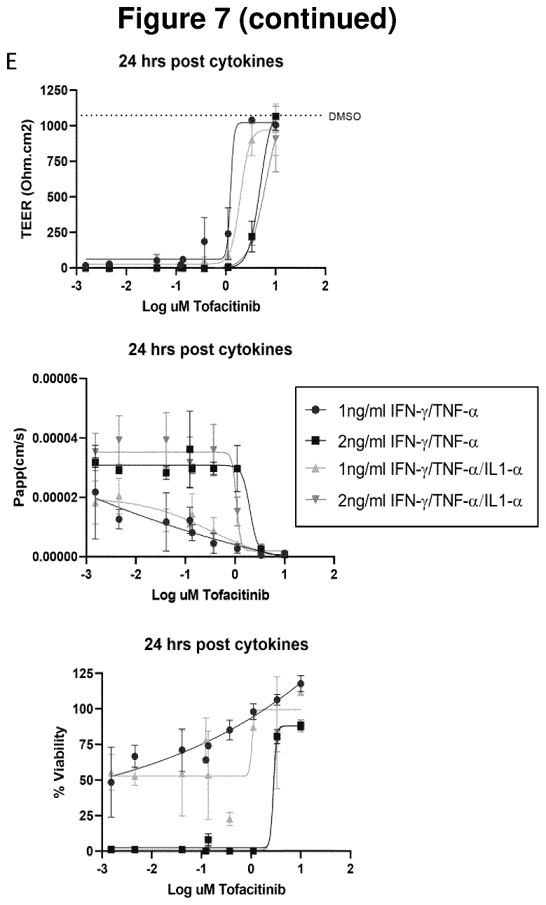

For screening purposes, we evaluated proinflammatory cytokine induced barrier function injury inhibition by tofacitinib on organoid-derived epithelium monolayers on 96 well Transwell plates. Single organoid cell suspension from colon organoids were seeded on transwells in CNM condition for 3 to 6 days, until epithelium monolayers were formed and TEER reached above 100 Ω·cm². At this point, the culture medium was changed to cCDM until epithelium monolayer barrier integrity further increased (TEER >1000 Ω·cm²). Subsequently, the monolayers were pre-treated with different tofacitinib concentrations for one hour, followed by proinflammatory cytokine cocktail IFN-γ/TNF-α/IL-1α or IFN-γ/TNF-α at end concentration of 1 (FIG. 7 A) and 2 ng/ml each. The effect of tofacitinib on barrier integrity was determined by TEER measurements after 5 and 24 hours. These measurements were normalized to the TEER value of the same Transwell before measurement, to correct for well-to-well TEER variability (FIG. 7B). Paracellular permeability was measured by the Lucifer Yellow (LY) permeability assay after 24 hours (FIG. 7C). Subsequently, monolayer cell viability was measured by the ATP Luminescent Assay, CellTiter-Glo 3D, to monitor for loss of barrier function due to cell death, as opposed to increased paracellular permeability. The results indicated that 2 ng/ml proinflammatory cytokine cocktail resulted in complete cell death after 24 hours, while epithelium monolayer viability treated with 1 ng/ml of triple and double proinflammatory cytokine cocktail combination decreased to 20 and 50 percent, respectively. (FIG. 7D).

Combinatorial Proinflammatory cytokine (IFN-γ/TNF-α/IL-1α or IFN-γ/TNF-α) treatment of colon epithelium monolayers, final concentration 1 and 2 ng/ml each, resulted in reduced and total loss of barrier integrity, after 5 and 24 hours, respectively. Pre-treatment of epithelium monolayers with increasing concentration of tofacitinib maintained barrier function integrity at concentrations above 3 μM for both cytokine combinations (FIGS. 7A and 7B). In concordance with TEER, apparent permeability of LY also was reduced with increased concentrations of tofacitinib above 3 μM (FIG. 7C). Cell viability measurement indicated that while proinflammatory cytokine cocktails were lethal to epithelium monolayers at 2 ng/ml end concentration, 1 ng/ml was better tolerated. However, tofacitinib pre-treatment, at concentrations above 3 μM, prevented cytokine induced cell death (FIG. 7D). The experimental condition above were reproduced by repeating the same experiment with same normal colon-derived epithelium monolayer and extended to normal ileum-derived organoid epithelium monolayers from the same donor, as in the previous experiment.

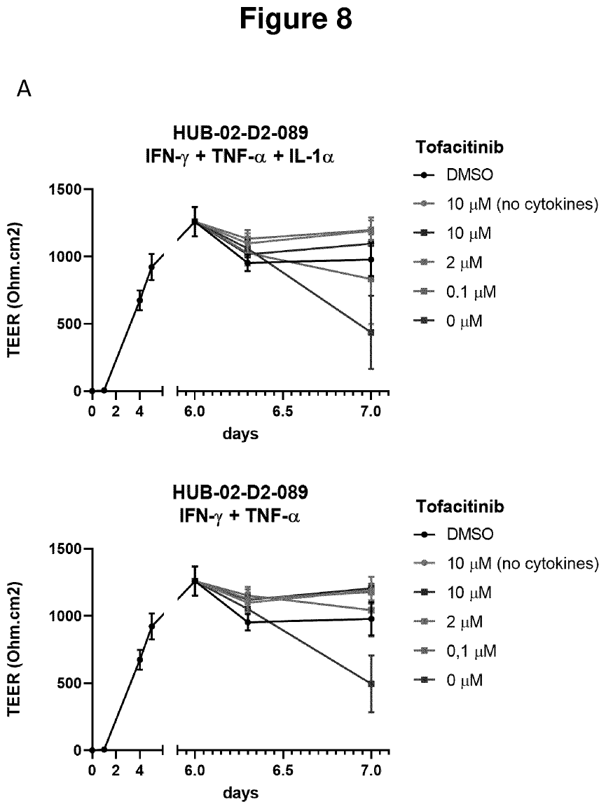

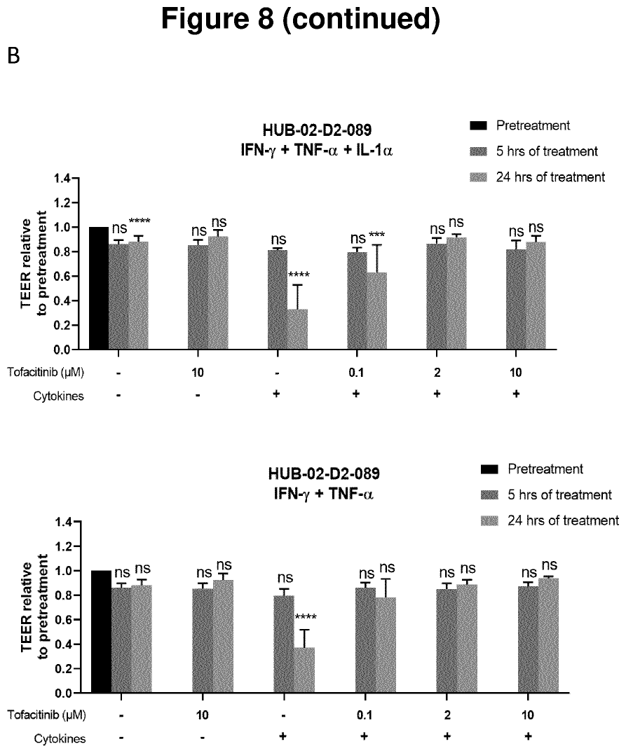

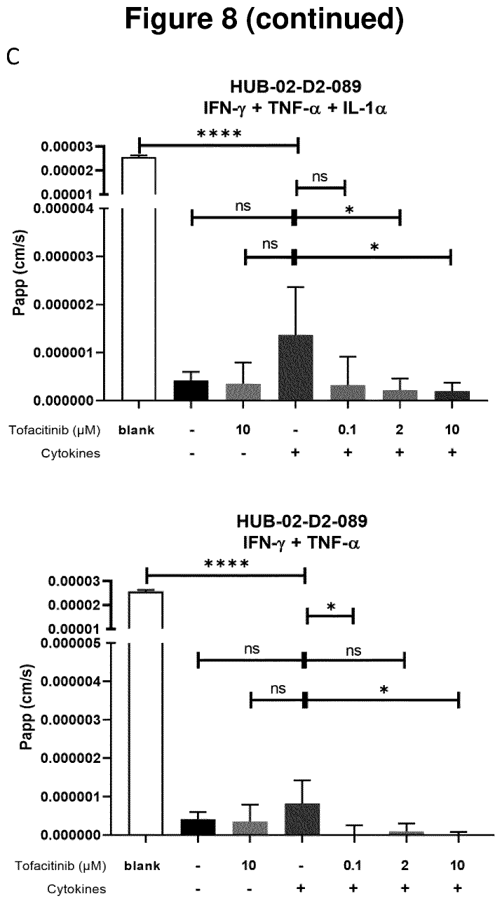

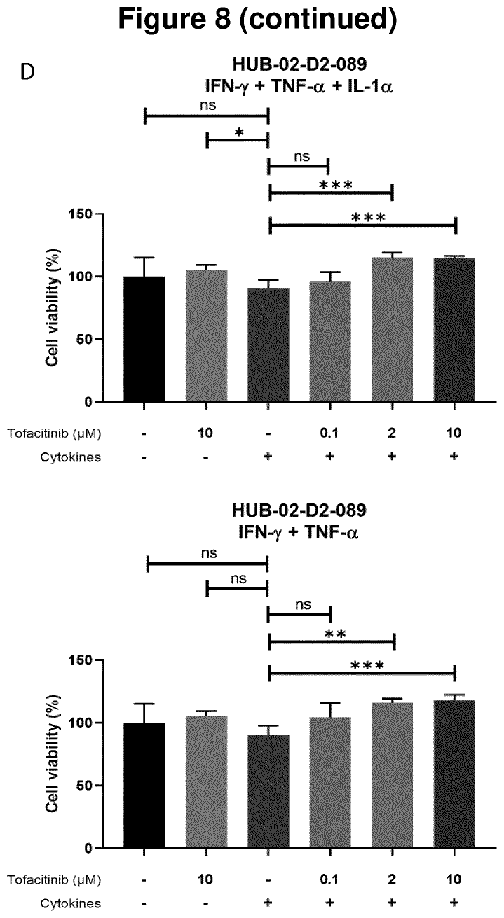

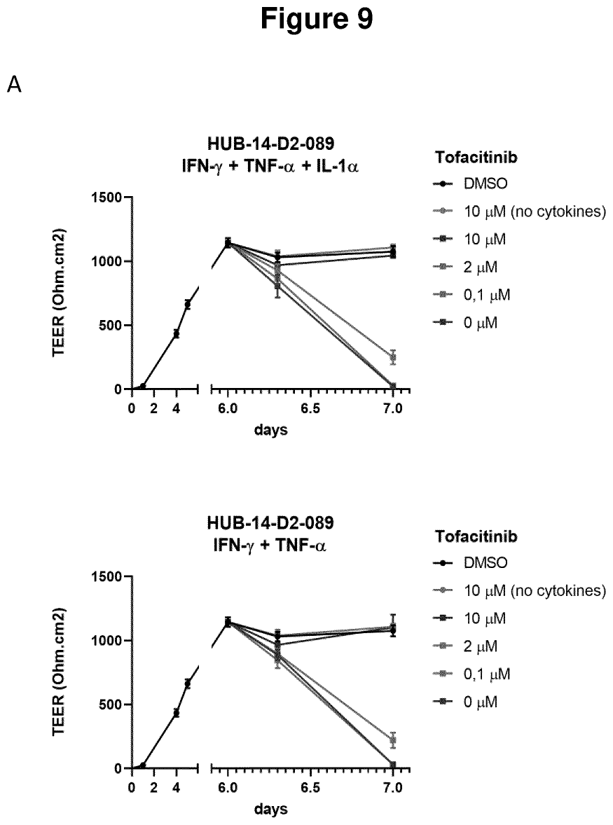

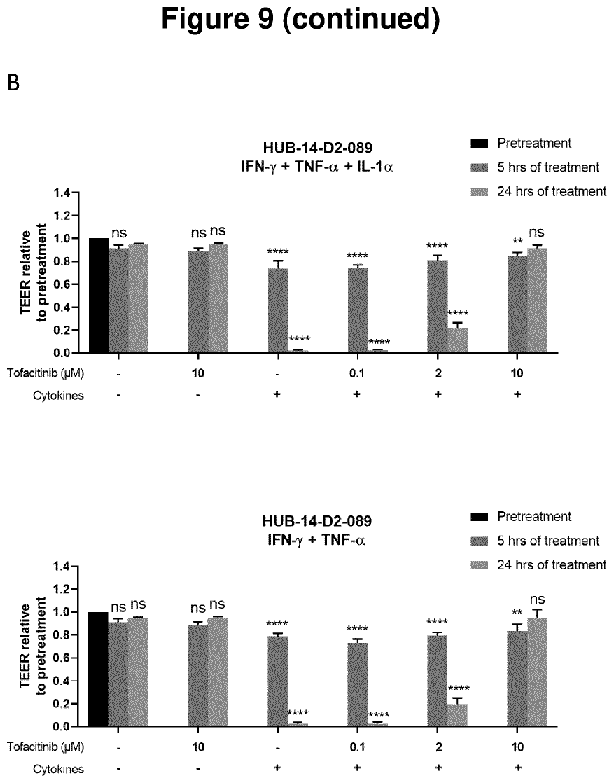

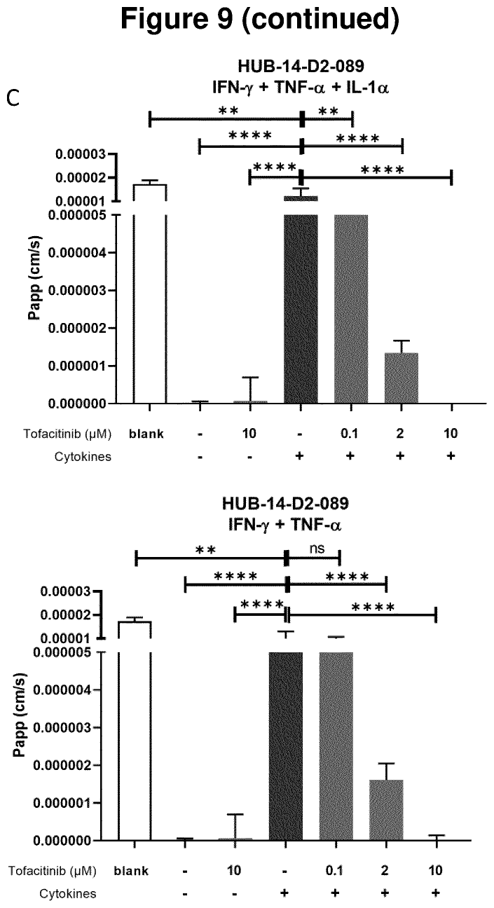

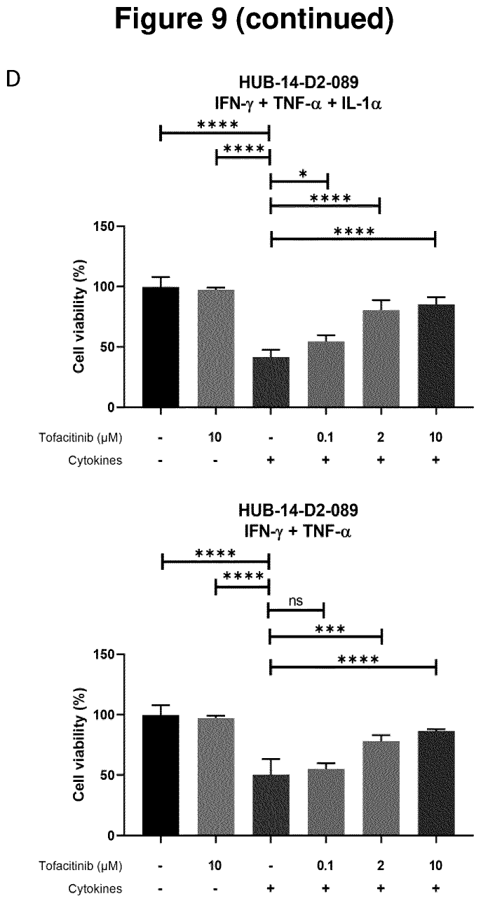

The epithelium monolayers were pre-treated with high (10 μM), around EC₅₀ (2 μM) and low (0.1 μM) tofacitinib concentrations (FIG. 7E and Table 6) an hour before barrier injury induction, using combinatorial proinflammatory cytokines at 1 ng/ml each (FIGS. 8 and 9). To address IFN-γ specificity in inducing barrier injury, TNF-α/IL-1α combination (data not shown) was included in addition to IFN-γ/TNF-α/IL-1α and IFN-γ/TNF-α.

TABLE 6
Summary of TEER, permeability, and cell viability
data in response to proinflammatory cytokines.

| | IFN-γ/TNF-α | | IFN-γ/TNF-α/IL-1α | |
|---|---|---|---|---|
| | 1 ng/mL | 2 ng/mL | 1 ng/mL | 2 ng/mL |
| TEER | ~1.246 | 4.909 | 1.958 | 6.382 |
| Permeability | ND | 2.004 | 0.3159 | ~1.066 |
| Cell Viability | ND | ~2.824 | ~1.042 | ~2.842 |

Abbreviations: ND (no data).

In colon-derived organoid epithelium monolayers, similarly to previous 10 experiments, the epithelium barrier integrity was compromised by both combination of IFN-γ/TNF-α/IL-1α and IFN-γ/TNF-α after 24 hours. However, combinatorial TNF-α/IL-la treatment caused milder barrier function injury (26% reduction of TEER value for TNF-α/IL-1α compare to 67 and 63% for IFN-γ/TNF-α/IL-1α and IFN-γ/TNF-α, respectively) that was not inhibited by highest tofacitinib concentration used (FIG. 9; TNF-α/IL-1α data not shown). This result underlined IFN-γ and tofacitinib specificity in inducing/inhibiting barrier function injury at the concentration and time point used. Tofacitinib pre-treatment partially protected colon organoid epithelium monolayers from cytokine induced barrier injury at 0.1 μM and completely at 2 μM final concentration (FIGS. 8A and 8B). Apparent permeability was not compromised significantly when colon epithelium monolayers were treated with 1 ng/ml of any cytokine combination used in this experiment. There was only a slight increase in apparent permeability, when the monolayers were not pre-treated with tofacitinib, followed by IFN-γ/TNF-α/IL-1α and IFN-γ/TNF-α. Cell viability measurements also mirrored the LY permeability results (FIGS. 8C and 8D).

Ileum-derived organoid epithelium monolayers seemed to be considerably more sensitive to IFN-γ/TNF-α/IL-1α and IFN-γ/TNF-α treatment, since they completely lost barrier integrity after 24 hours (FIGS. 9A and 9B). However, unlike the other two cytokine combinations containing IFN-γ, TNF-α/IL-1α treatment did not compromise ileum barrier integrity (data not shown), which again, similar to colon, underlined IFN-γ requirement for barrier function injury. Tofacitinib pre-treatment protected ileum epithelium monolayers at higher concentration compared to colon, 10 versus 2 μM (FIGS. 9A and 9B). In concordance with barrier integrity observation, LY permeability and cell viability increased and decreased, respectively, by IFN-γ containing cytokine cocktails only and became impermeable again by 10 μM tofacitinib pre-treatment.

Altogether, we concluded that organoid-derived epithelium monolayers were established from different GI tract regions on 96 well transwells. The epithelium monolayers were driven to different cell fates and used in inducing barrier function injury assays with screening purposes by measuring barrier integrity, permeability, and cell viability.

### Example 4. Validation of the Robustness of a Barrier Function Assays with Intestinal Organoid-Derived Monolayers

### Barrier Function Assay Reproducibility in IBD-PDO Derived Epithelium Monolayers

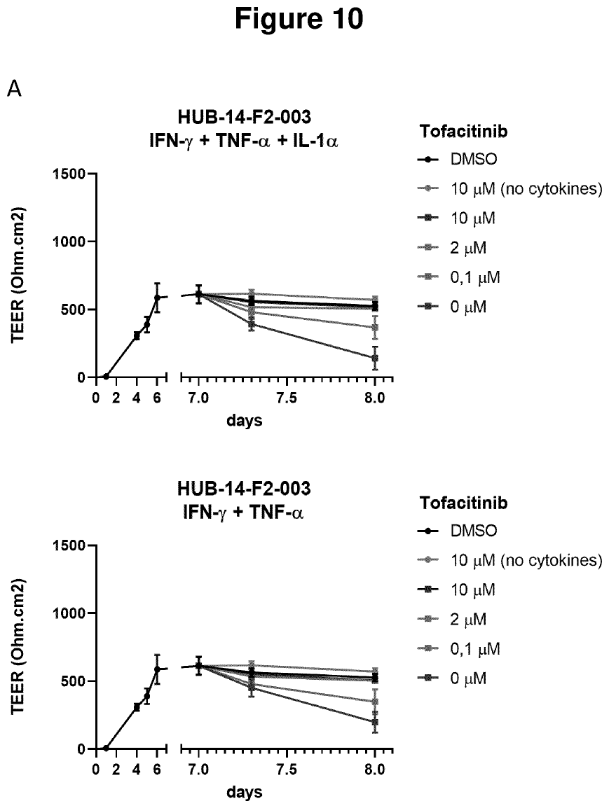

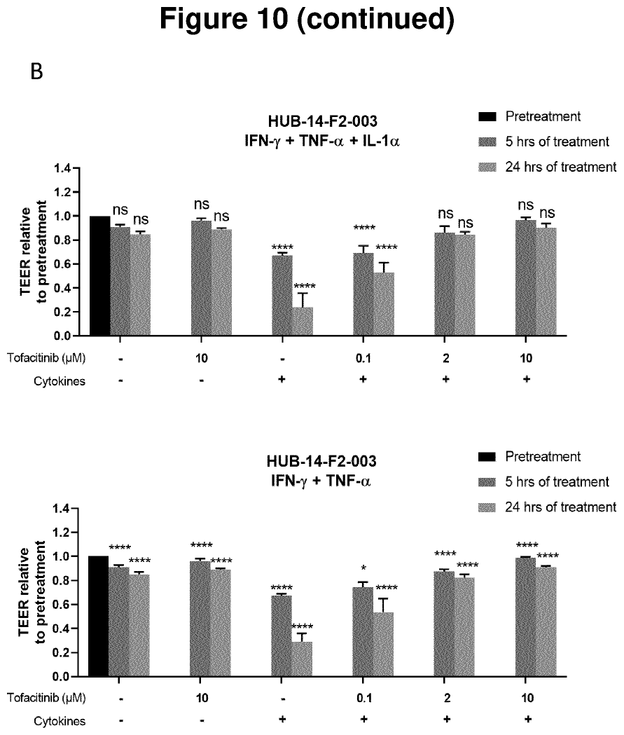

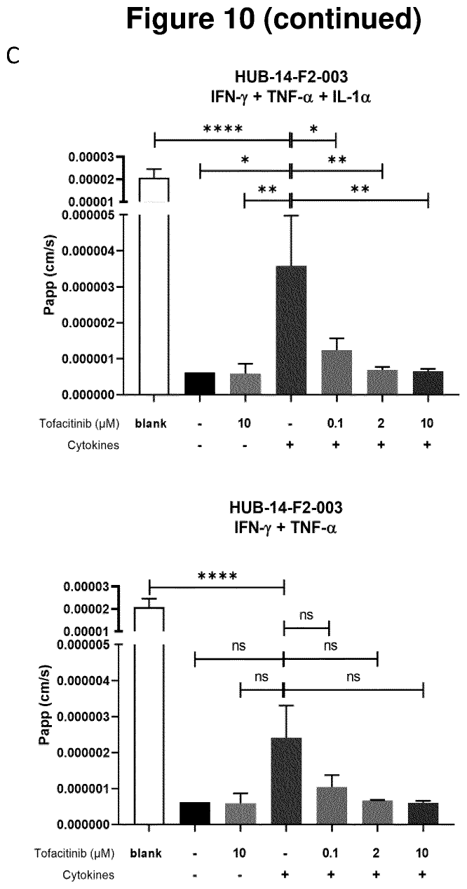

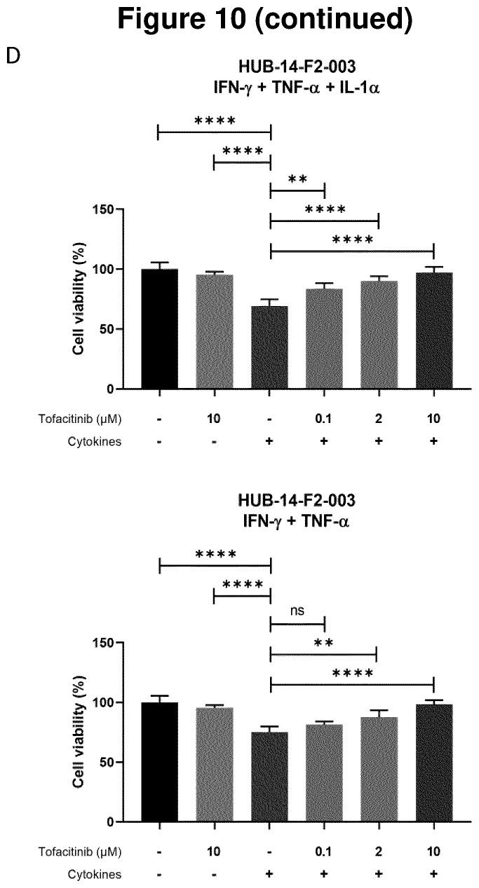

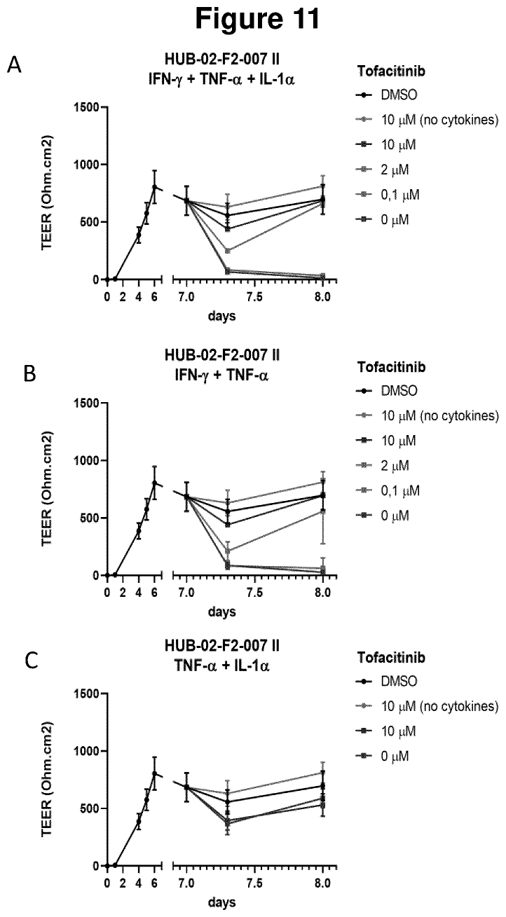

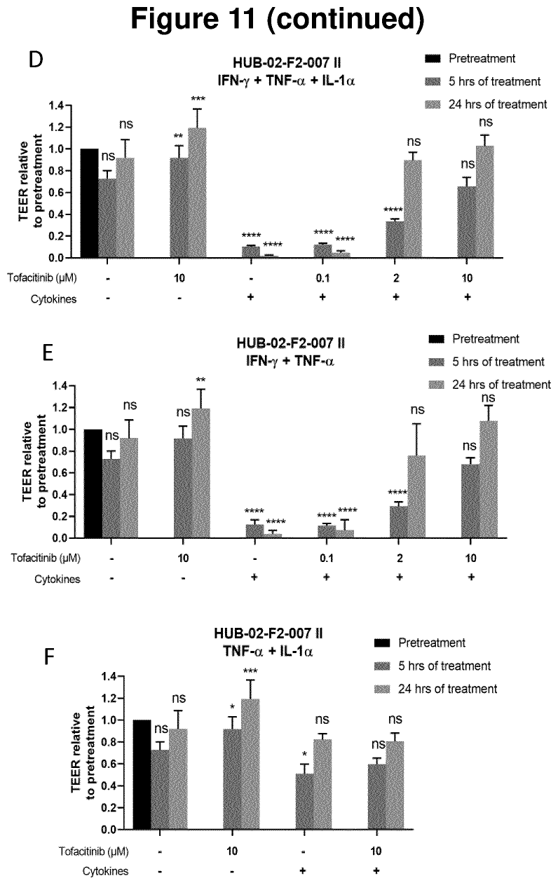

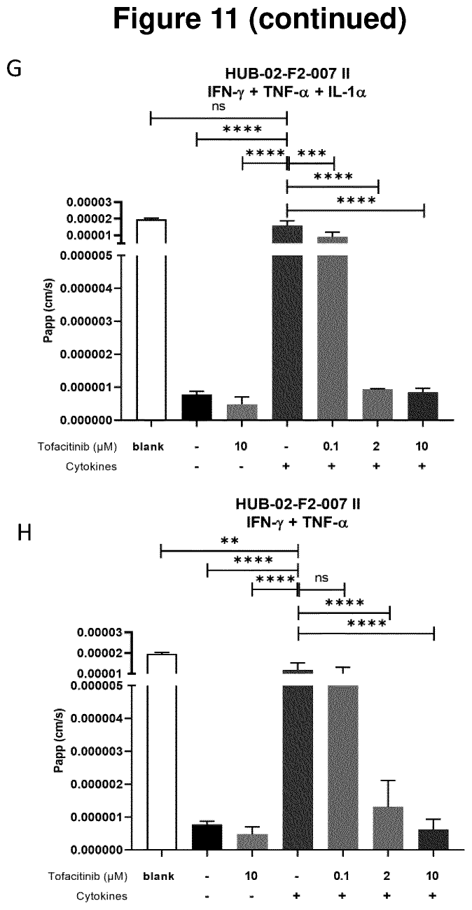

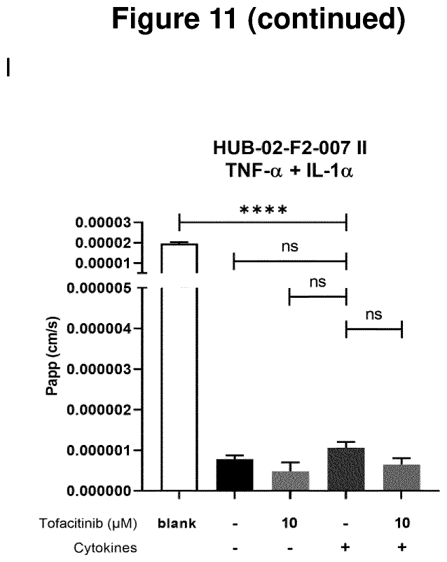

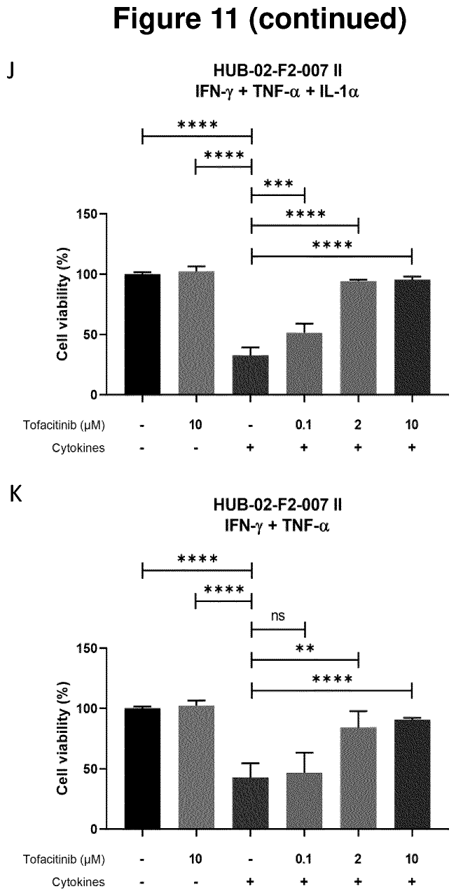

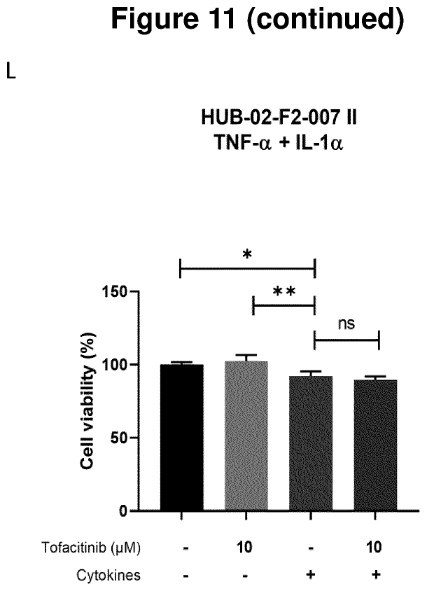

IBD patient-derived organoid (IBD-PDO) monolayer cultures from ileum, proximal and distal colon were established following the same protocols used in previous experiments. The monolayers were pre-treated with 0.1, 2 and 10 μM tofacitinib one hour before inducing barrier injury using 1 ng/ml of either proinflammatory cytokine combinations of IFN-γ/TNF-α/IL-1α, IFN-γ/TNF-α, or TNF-α/IL-1α for 24 hours. Their barrier integrity was measured at 5 and 24 hours followed by LY permeability and cell viability performed (FIGS. 10, 11).

IBD-PDO ileum epithelium monolayers did not reach the TEER value of above 1000 Ω/cm², the TEER had increased once the culture conditions were changed to cCDM. The epithelium monolayers had similar sensitivity to IFN-γ/TNF-α/IL-1α and IFN-γ/TNF-α, which were inhibited by tofacitinib pre-treatment in a dose response manner. Barrier function remained unchanged in response to TNF-α/IL-1α treatment in IBD-PDO derived ileum epithelium monolayer (data not shown), which again underlined IFN-γ and tofacitinib specificity in inducing and inhibiting barrier function injury, respectively (FIGS. 10A and 10B). The LY permeability and cell viability experiments agreed with barrier integrity damage and compromised by IFN-γ/TNF-α/IL-1α and IFN-γ/TNF-α and inhibited by tofacitinib in a dose responsive manner (FIGS. 10C and 10D).

IBD-PDO proximal colon epithelium monolayers were less sensitive to IFN-γ/TNF-α/IL-1α and IFN-γ/TNF-α, as relative TEER values in cytokine treated conditions after 5 hours treatment dropped relatively to 0.59 and 0.66 (data not shown) as compared to 0.24 and 0.29 in IBD-PDO derived ileum epithelium monolayers and 0.11 and 0.12 in IBD-PDO derived distal colon epithelium monolayers. The induced barrier integrity damage was completely restored after 24 hours in monolayers pre-treated with higher than 0.1 μM tofacitinib (data not shown). The LY permeability and cell viability experiments indicated that induced damages were not enough to increase monolayer permeability and therefore the effect of tofacitinib on this readout could not be assessed. Altogether, the data suggested that IBD-PDO proximal colon epithelium monolayers were not sensitive to proinflammatory cytokines and that increased cytokine concentrations were required to resolve tofacitinib dose response inhibitory impact.

IBD-PDO derived distal colon epithelium monolayers were the most sensitive, with TEER values decreasing to 0.11, 0.12 and 0.51 relative to untreated controls in response to IFN-γ/TNF-α/IL-1α, IFN-γ/TNF-α and TNF-α/IL-1α, respectively, as compared to 0.24 and 0.29, 0.95 for IBD-PDO derived ileum monolayers and 0.59, 0.66, 0.87 for IBD-PDO derived proximal colon monolayers. Epithelium barrier integrity was lost after 5 hours in monolayers treated with IFN-γ/TNF-α/IL-1α and IFN-γ/TNF-α and compromised with TNF-α/IL-1α (data not shown). Unlike other organoid monolayer cultures, the induced damage in IBD-PDO derived distal colon monolayers was not completely inhibited even with highest tofacitinib concentration at 5 hours. The damage was restored after 24 hours, indicating the highest tofacitinib concentration protected the monolayer from excessive damage, giving the chance to the organoid cells for restoring the barrier after 24 hours. Barrier function integrity in response to TNF-α/IL-1α was also reduced in the IBD-PDO derived distal colon monolayer, but not inhibited or restored with highest tofacitinib concentration (data not shown). The LY permeability and cell viability experiments agreed with barrier integrity damage which were compromised by IFN-γ/TNF-α/IL-1α and IFN-γ/TNF-α and inhibited by tofacitinib in a dose responsive manner (FIG. 11G-L). It is worth mentioning that the IBD-PDO derived distal colon organoid culture is carrying ATG16L1 T300A homozygote mutation, two NOD2 and IL23R IBD predisposition SNPs. Whether the genetic susceptibility SNPs are involved in their higher responses to inflammatory stimuli remains to be determined.

Altogether, these data indicated that epithelium monolayers can be generated from IBD-PDO and be used for barrier function studies in line with development of screening funnels for small molecule barrier modulators.

### Example 5. Human GI Tract Organoid Epithelium Monolayer Establishment

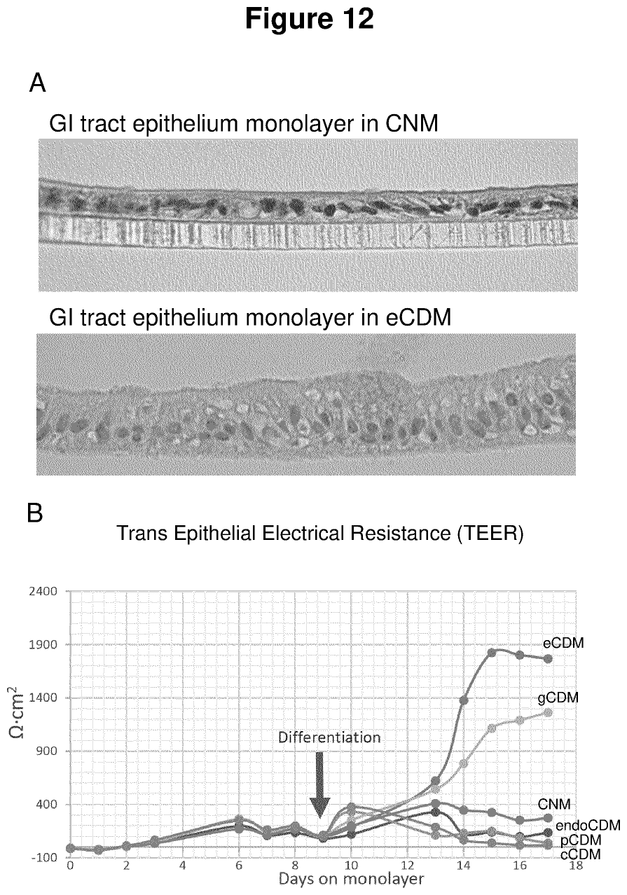

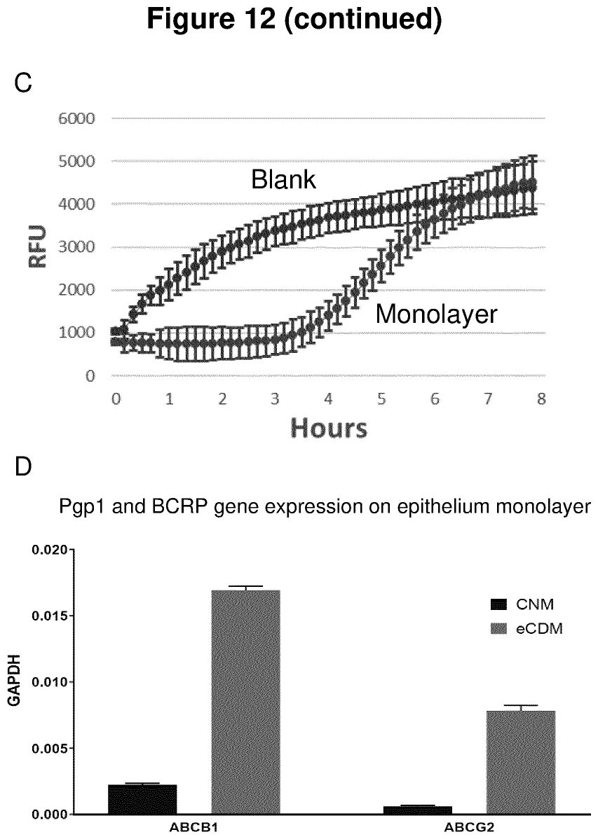

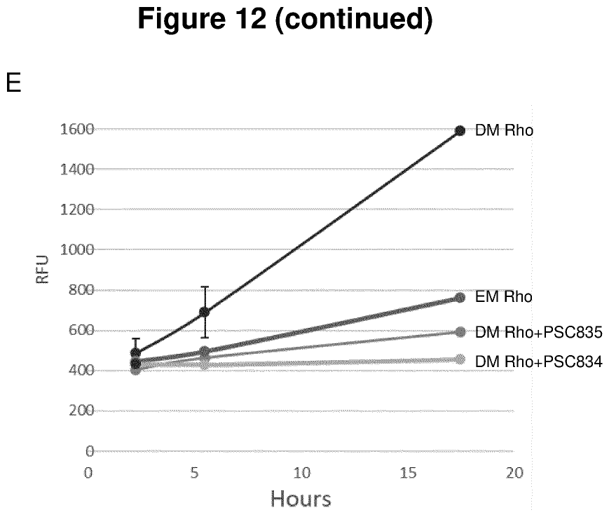

Permeability and transport of different compounds are studied by either cell lines grown on a Transwell system forming an epithelium monolayer or primary intestinal epithelium tissue mounted on Ussing chamber. While many different enzymes and transporters are aberrantly expressed in adenocarcinoma cell lines such as Caco-2 cells, the Ussing chamber is very demanding. Organoid-derived epithelial monolayers would combine the ease of a cell line and the accuracy of primary tissue and therefore we sought to establish such a monolayer using human duodenal and colon organoids. This was achieved by digestion of human duodenum organoids to single cells and seeding them on a Transwell membrane and differentiating them using eCDM. Similar to organoids, epithelium monolayer cross section H&E staining on CNM contains simple squamous epithelium appearance that is transformed to simple columnar epithelium four days after differentiation (FIG. 12A). Epithelium monolayer integrity was evaluated by Trans Epithelial Electrical Resistance (TEER) which reached between 100 to 200 Ω·cm² on day 5-6 and stayed stable until day nine when they were differentiated using different differentiation media and followed by TEER measurement for additional eight days. Among different differentiation conditions, eCDM and gCDM displayed increased TEER value of above 1000 Ω·cm² indicating tight monolayer formation which stayed stable for three days (FIG. 12B). Since eCDM induces enterocyte differentiation and adequate expression of physiologically relevant proteins such as alkaline phosphatase and mucus in organoids, and tighter barrier function on epithelium monolayers, eCDM was selected for further experiments. Next, we evaluated day four enterocyte differentiated epithelium monolayers paracellular permeability by passive diffusion of Lucifer Yellow (LY) from apical to basolateral side and fluorescence measurement for up to eight hours. Enterocyte-differentiated epithelium monolayer stay impermeable for up to four hours (FIG. 12C). In addition to intact paracellular permeability, active transport is another important GI tract epithelium function for transport of different compounds. P-glycoprotein 1 (permeability glycoprotein, Pgp1) also known as multidrug resistance protein 1 (MDR1) or ATP-binding cassette sub-family B member 1 (ABCB1) is an important protein extensively expressed in the apical membrane of intestinal epithelium where it pumps xenobiotics (such as toxins or drugs) back into the intestinal lumen. Breast cancer resistance protein (BCRP or ABCG2) is another important xenobiotic transporter expressed at the apical membrane of intestinal epithelium. Expression of these two important xenophobic transporters increases more than ten folds after differentiation of epithelium monolayers toward enterocytes (FIG. 6D). To evaluate Pgp1 functionality, Rhodamine 123, a Pgp1 substrate, was applied to the basal side of the Transwell and its active transport was measured at the apical side of epithelium monolayer. Inhibition of Pgp1 by specific inhibitor, PSC833, results in reduced Rhodamine 123 efflux. The result of this experiment confirms epithelium monolayers derived from human GI tract organoids have transport function, and that it is increased upon enterocyte differentiation, in line with increased expression of transporter proteins (FIGS. 12D and E). This transport functionality of the monolayers is Pgp1 specific as it is inhibited by PSC833 and functional for up to 18 hours.

To further characterise human GI tract epithelium monolayers, human duodenum and colon organoid-derived monolayers cultured on Transwell plates were differentiated and stained to detect the expression of several key proteins (FIG. 13). Similar to previous observations with 3D organoids, duodenum and colon organoid-derived monolayers form columnar epithelium and lose their proliferative Ki67 positive cells upon differentiation (FIG. 13). Goblet cells are clearly more visible in the differentiated colon monolayer than in the duodenum monolayer in H&E-stained specimens, with increased mucus production as confirmed by Alcian blue staining (FIG. 13

All together, these results indicate organoids can be used to establish human epithelium monolayers from different GI tract regions. These epithelium monolayers can be differentiated to enterocytes, are polarized, impermeable with barrier and transport function, and therefore can be used for compound permeability, metabolism and transport studies.

### Example 6. Polarisation of Human GI Tract Organoid Epithelium Monolayers

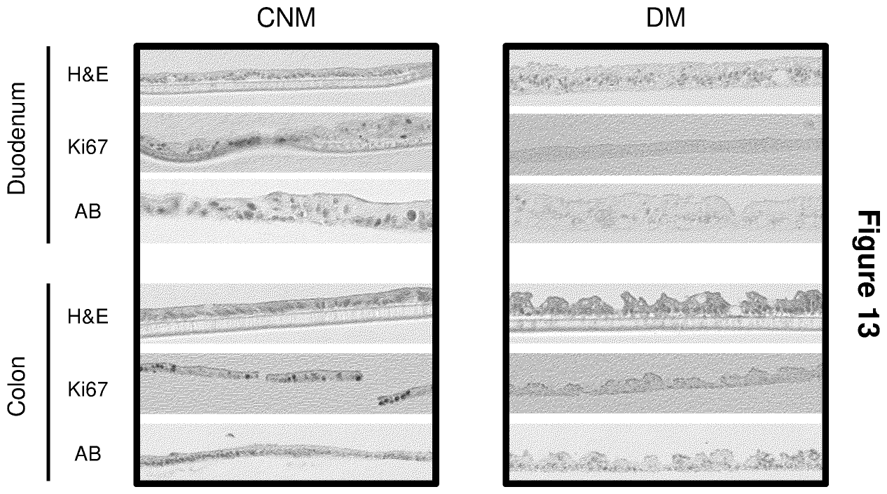

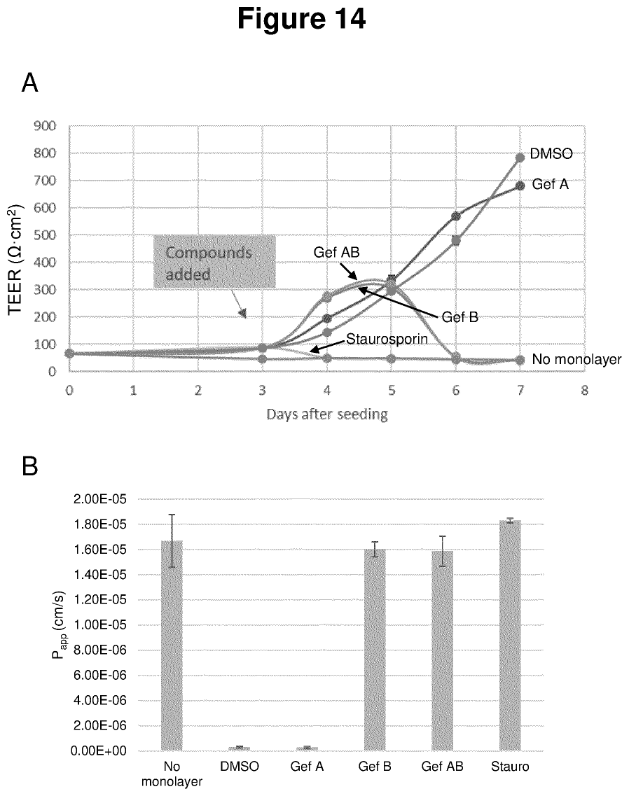

Human gastro-intestinal tract organoid-derived monolayers were seeded and differentiated in eCDM as described herein, and treated with DMSO, staurosporin or Gefitinib on day 3 after seeding. Gefitinib was applied to the apical compartment, the basolateral compartment, or both compartments. TEER (FIG. 14A) and Lucifer yellow permeability (FIG. 14B) were measured as in the preceding Examples.

Gefitinib is an EGFR inhibitor which results in growth inhibition. The present example shows that the integrity of organoid-derived epithelial monolayers is compromised only when Gefitinib is added to the basolateral compartment. Since EGFR is predominantly localised to the basolateral cell surface in human epithelial tissue, loss of barrier integrity of the monolayers upon basolateral treatment with Gefitinib demonstrates that the monolayers are polarised and leak-tight.

### Example 7. Establishment and Characterisation of Lung Organoid Monolayers

### Monolayer Establishment and Differentiation

Human lung organoids from three different donors (lung-A, lung-B, lung-C) were passaged at high density (ratio ˜1:2) three to four days prior to monolayer preparation. On the day of harvesting, medium from the well was used to break the organoid drops and organoids were washed once in DMEM supplemented with 0.1% BSA and Pen/Strep, centrifuged at 450×g for 5 minutes at 8° C., and washed once in PBS without Mg²⁺ and Ca²⁺. Organoids were digested to single cells and small clumps (2-4 cells) using Accutase by incubating in the water bath and checking and resuspending the material every 5 minutes. Single cells were washed with Advanced DMEM/F12, supplemented with 2 mM GlutaMax, 10 mM HEPES and Pen/Strep, centrifuged at 450×g for 5 minutes and 8° C. twice. Cells were passed through a pre-wetted 40 μm cell strainer and resuspended in lung expansion medium (LuM; Advanced DMEM/F12, 1% HEPES, 1% GlutaMAX, 1% penicillin/streptomycin, 1.25 mM N-Acetylcysteine, 1×B27 supplement, 25 ng/ml FGF-7, 100 ng/ml FGF-10, 5 mM Nicotinamide, 50 μg/ml Primocin, 250 ng/ml Rspondin-3, 500 nM SB202190 (p38i), 5 μM Y-27632 (Rho Kinase inhibitor), 500 nM A83-01, 2% Noggin UPE) with a density of 2 million cells/ml supplemented with 10 μM RhoKI. In parallel with organoid preparation, Corning® HTS Transwell® 96 well permeable supports, polyester membrane with 0.4 μm pore size inserts were placed in the corresponding receiver plate. Matrigel was diluted 40× with ice-cold PBS (with Ca2+ and Mg2+). Apical surfaces of transwells were either left uncoated, or coated by applying 65 μl of 2.5% Matrigel for 1 hour at 37° C. After carefully removing PBS from the coated inserts, 300 μL of LuM was added to the basolateral compartment. Transwells were seeded by adding 100 μl of cell suspension at various cell densities (30,000-250,000 cells/transwell) on the apical compartment. Plates were incubated at 37° C. and 5% CO₂ and medium was refreshed three times a week.

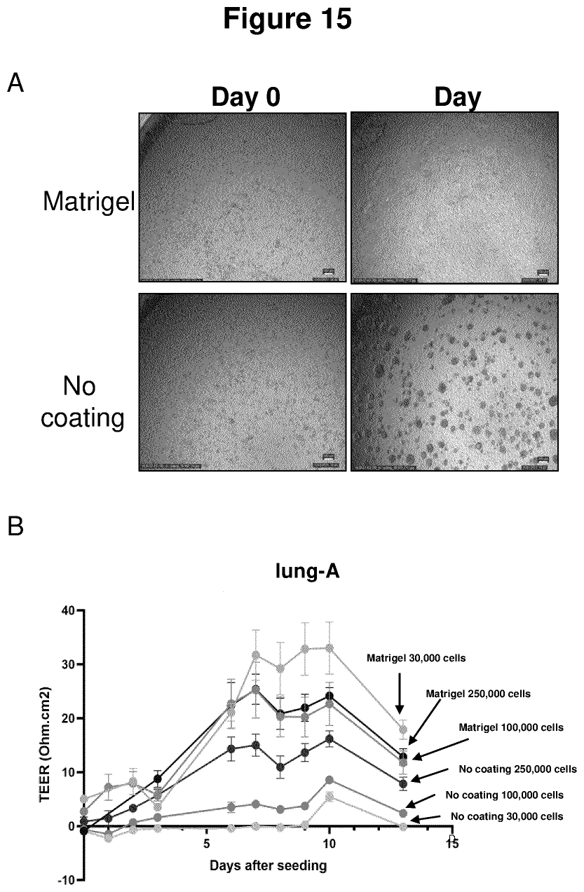

The Matrigel coating was essential for formation of monolayers of cells derived from lung organoids (FIG. 15A). For cell monolayers derived from lung organoids, the inventors surprisingly found that seeding the Matrigel-coated Transwells with a lower number of cells (30,000 cells) resulted in higher TEER values than seeding the Matrigel-coated Transwells with a higher number of cells (e.g. 100,000 cells or 250,000 cells) (FIG. 15B). The higher TEER values for lower cell seeding densities (e.g. 30,000 cells) were more notable, in particular, 8 days after seeding the cells. All of the cultures seeded onto Transwells without the Matrigel coating displayed lower TEER values than cultures seeded onto Transwells with the Matrigel coating. Therefore, Matrigel coating and 40,000 cells were used for all subsequent experiments on cells derived from lung organoids.

The following culture conditions were assessed: lung expansion medium (LuM), and change of the medium to ciliation lung medium (cLuM; Advanced DMEM/F12, 1% HEPES, 1% GlutaMAX, 1% penicillin/streptomycin, 1.25 mM N-Acetylcysteine, 1×B27 supplement, 25 ng/ml FGF-7, 100 ng/ml FGF-10, 5 mM Nicotinamide, 50 μg/ml Primocin, 250 ng/ml Rspondin-3, 500 nM SB202190 (p38i), 5 μM Y-27632 (Rho Kinase inhibitor), 10 μM DAPT, 10 ng/ml BMP4) on day 3, 4, or 8 after seeding. The measured TEER of the cultures typically increased after the change of medium to cLuM, and as the cell monolayers became more confluent.

Lung monolayers were grown in liquid-liquid interface (LLI) and air-liquid interface (ALI) format. Liquid-liquid interface (LLI) and air-liquid interface (ALI) cultures were assessed to determine optimal experiment settings for lung monolayer formation. On day 13 when monolayers were formed, ALI cultures were initiated by removing medium from the apical compartments of the transwells so that the monolayers would be directly exposed to air. The cultures were kept for 11 days in this condition, until day 24. TEER values were measured to monitor the integrity of the monolayers. As a control, LLI conditions were maintained in parallel by leaving the medium in both apical and basolateral compartments for further 11 days, until day 24. TEER values were measured to monitor the integrity of the monolayers.

The morphology, barrier function, marker expression (the present example) and transport function (Example 8) of the lung organoid-derived monolayers was assessed 4 or 8 days after changing the cell culture medium to cLuM.

### Morphology

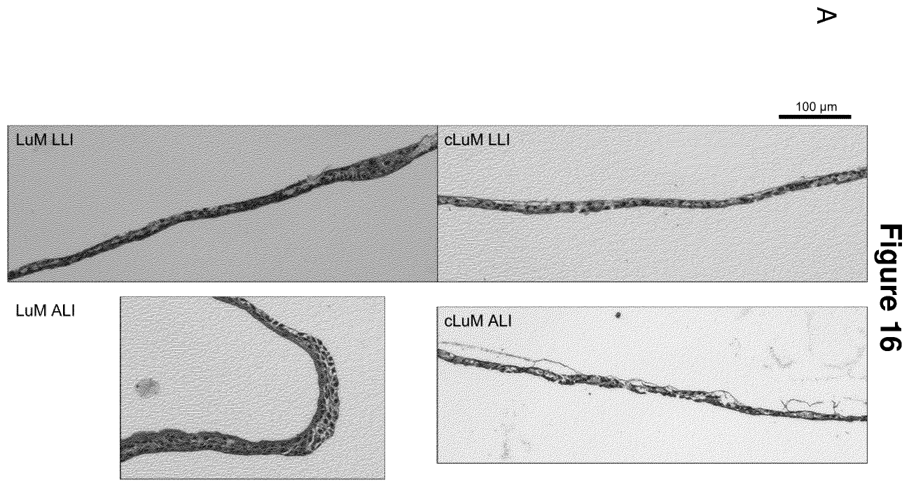

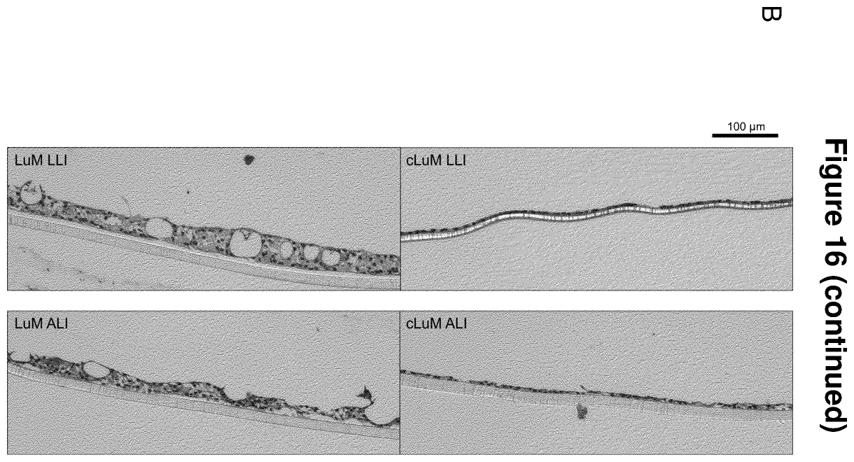

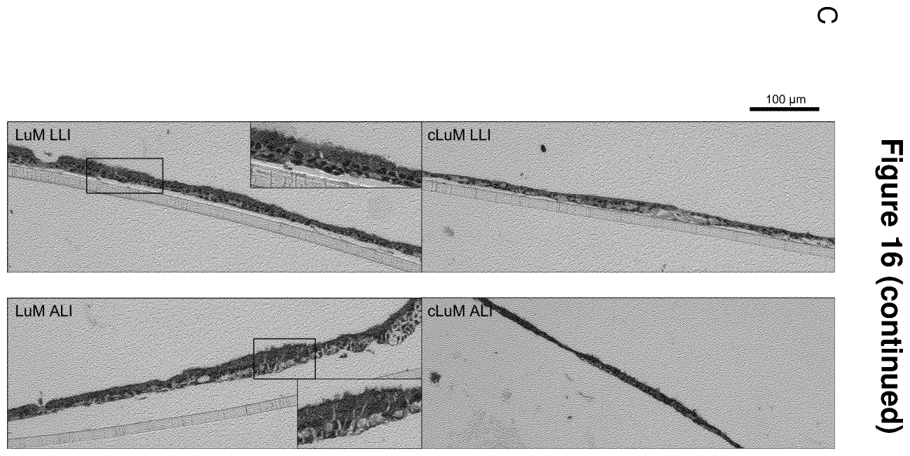

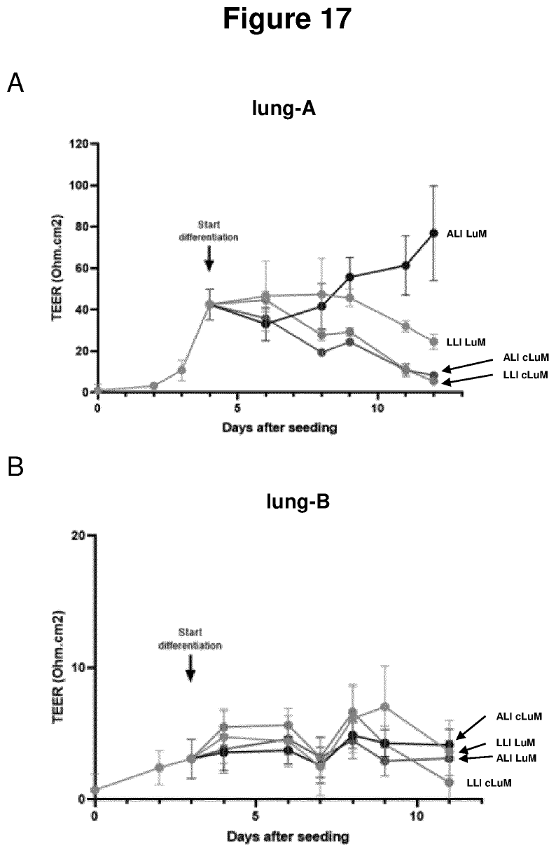

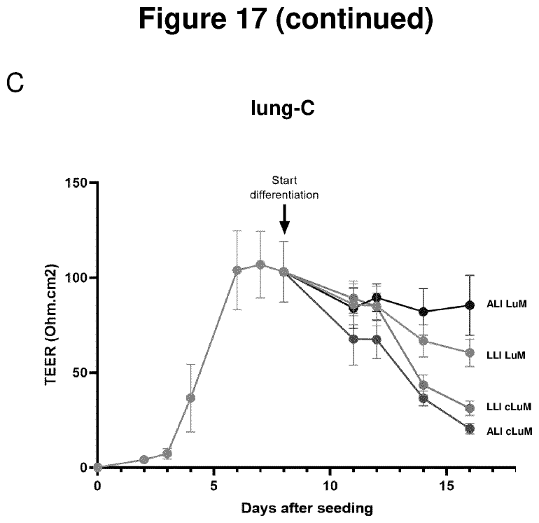

The morphology of the lung organoid monolayers was assessed using H&E staining, which revealed a monolayer of pseudostratified epithelial cells during both expansion (in LuM medium) and differentiation (in cLuM medium) (FIG. 16A-C). the lung organoid monolayers display a heterogenous cell population, which is reflected in their barrier properties (TEER and permeability, see below) and histological appearance. Small ‘bubbles’ are visible in the cell layers media which correlate with expression of alveolar markers (FIG. 16A, cLuM ALI; FIG. 17A LuM LLI and ALI), suggesting that the lung organoid monolayers are useful models for lung epithelium.

### Permeability

Permeability of the monolayers was assessed throughout the experiment by measuring TEER (FIG. 17A-C). The TEER values of the lung monolayers increased after seeding and remained at measurable values in different culture media (cLuM or LuM) and culture formats (ALI or LLI).

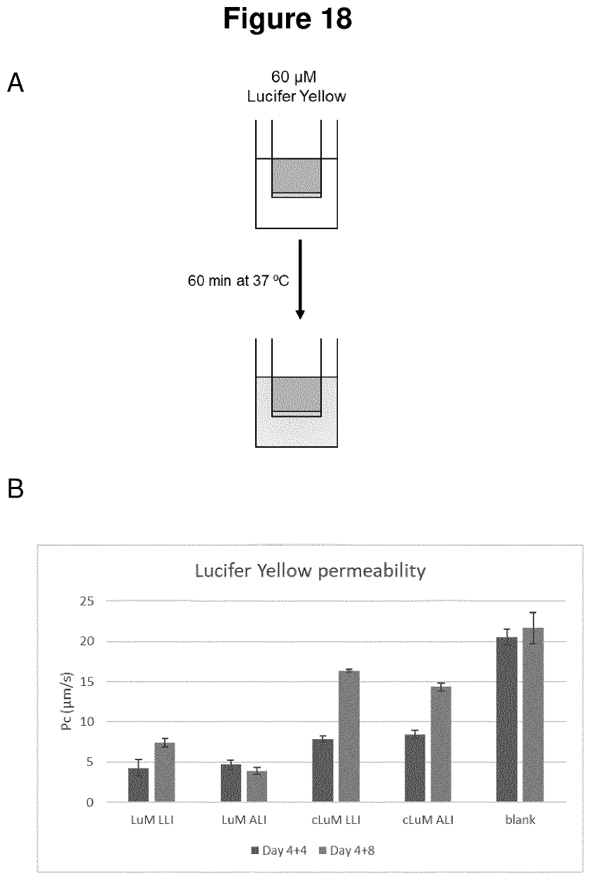

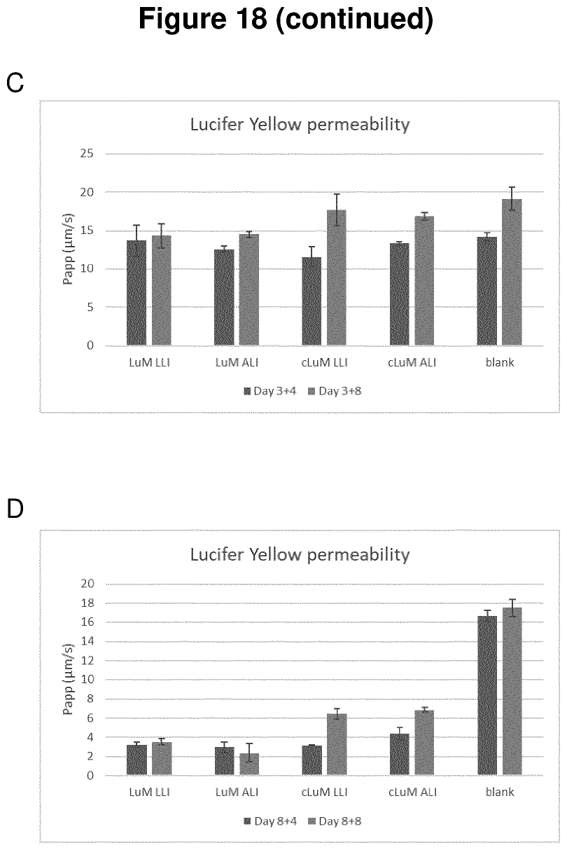

Permeability was also assessed using the lucifer yellow assay (FIG. 18A). Briefly, 60 μM lucifer yellow was added to the apical compartment. After a 60-minute incubation at 37° C., diffusion of lucifer yellow into the basolateral compartments was measured. The results are shown in FIG. 18 B-D. In general, lung monolayer cultures 15 showed lower permeability to lucifer yellow than control samples (blank).

### Marker Expression

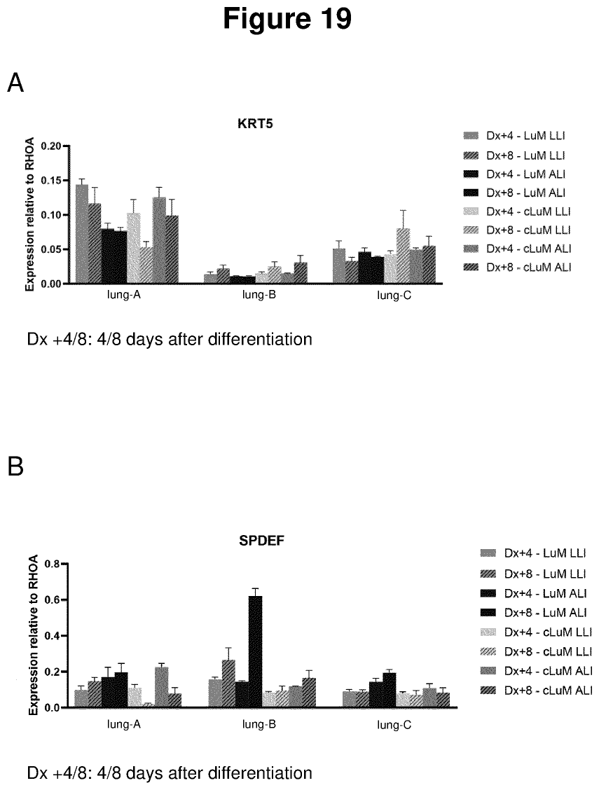

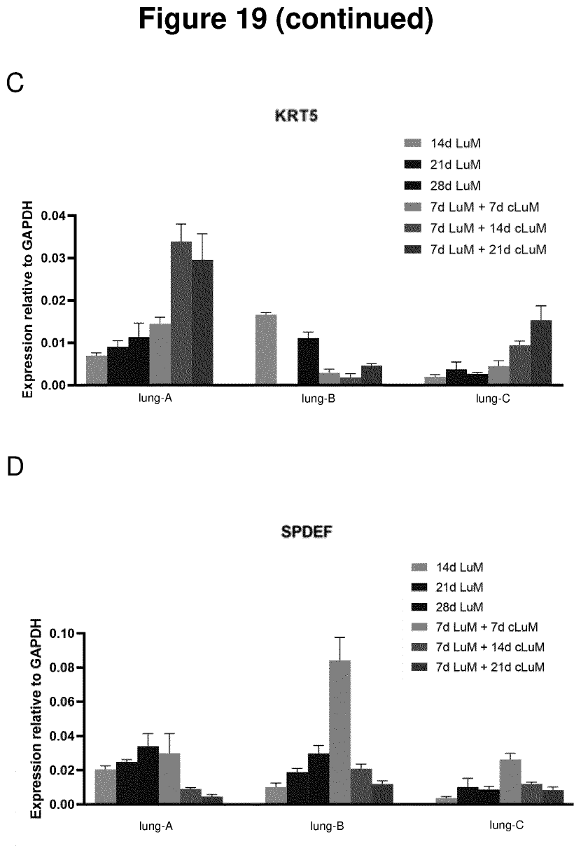

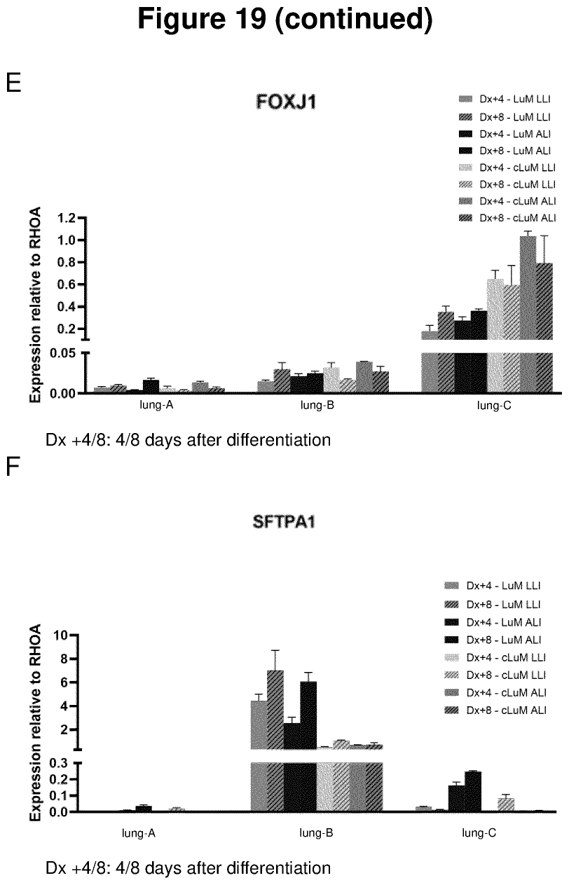

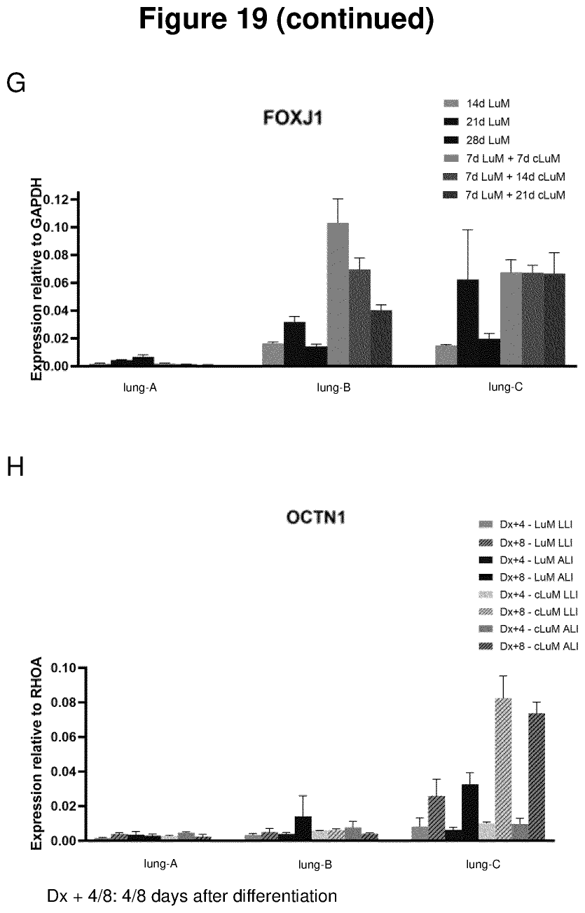

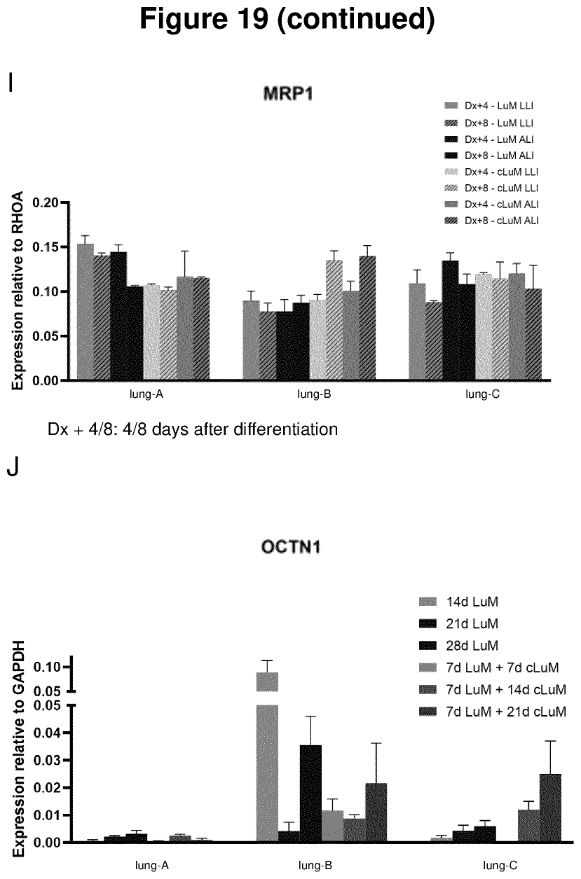

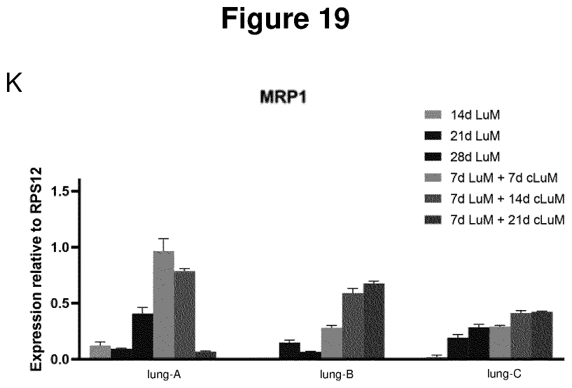

Expression of various lung markers and transporter proteins in the lung monolayers was measured using RT-qPCR. Expression was also assessed in the organoids which were used for seeding the monolayers. FIGS. 19A and B show expression levels of KRT5 and SPDEF in organoid monolayers. FIGS. 19C and D show expression levels of KRT5 and SPDEF in lung organoids. FIGS. 19E and F show expression levels of FOXJ1 and SFTPA1 in organoid monolayers. FIG. 19G shows expression levels of FOXJ1 in lung organoids. FIGS. 19H and I show expression levels of OCTN1 and MRP1 in organoid monolayers. FIGS. 19J and K show expression levels of OCTN1 and MRP1 in lung organoids.

The lung organoid monolayers were grown in LuM, or were differentiated in cLuM for 4 or 8 days. The lung organoids were cultured for various durations in LuM or cLuM as described. Expression of the following lung markers was assessed: KRT5 (lung basal cell marker), SPDEF (goblet cell marker), FOXJ1 (ciliated cell marker), and SFTPA1 (lung alveoli marker). Expression of the transporter proteins OCTN1 and MRP1 was also measured. The results are shown in FIG. 19.

Lung markers KRT5 and SPDEF were detected in both lung monolayers and lung organoids. Ciliated cell marker FOXJ1 was detected in one of the lung monolayers and two of the lung organoids, and lung alveoli marker SFTPA1 was detected in the lung-B culture sample (FIG. 19F), which correlated with the appearance of ‘bubble’ structures visible when lung monolayers were cultured in LuM media in both LLI and ALI formats (FIG. 16B). The Lung-C culture sample showed the most ciliation in histological images (FIG. 16C), and the ciliated cell marker FOXJ1 was highly expressed in this culture (FIG. 19E). The Lung-C culture sample also showed the highest expression of the OCTN1 transporter. Expression of the transporter OCTN1 was detected in both lung monolayers and lung organoids, and expression of the transporter MRP1 was detected in all of the lung monolayer and lung organoid samples, in all conditions.

### Example 8. Calcein Transport Assay in Lung Organoid Monolayers

This Example demonstrates the development of transport assays that allow measurement of transporter function of lung organoid monolayers through accumulation of fluorescent dyes in the monolayer. The lung monolayer lung-C was selected for the transport assays, on account of its tight barrier function (FIG. 17C and FIG. 18D).

### Calcein Transport Assay—Accumulation Assay

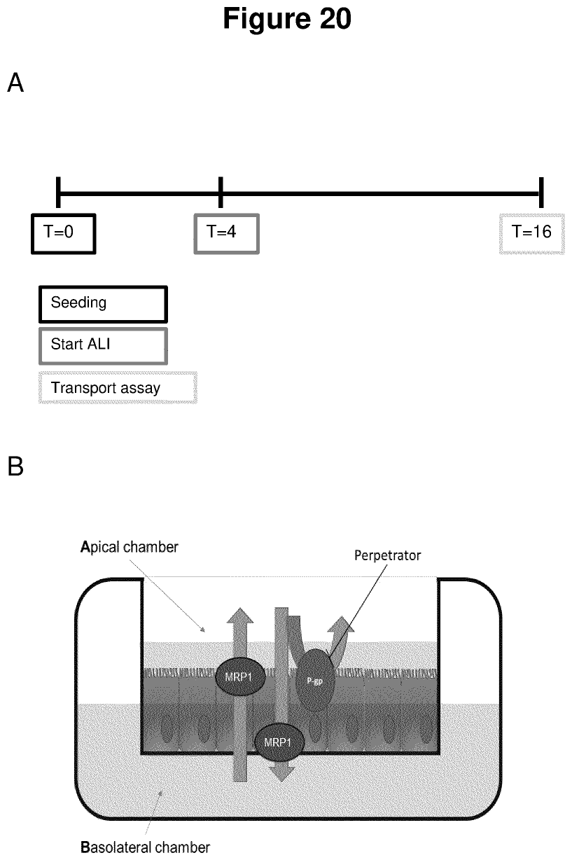

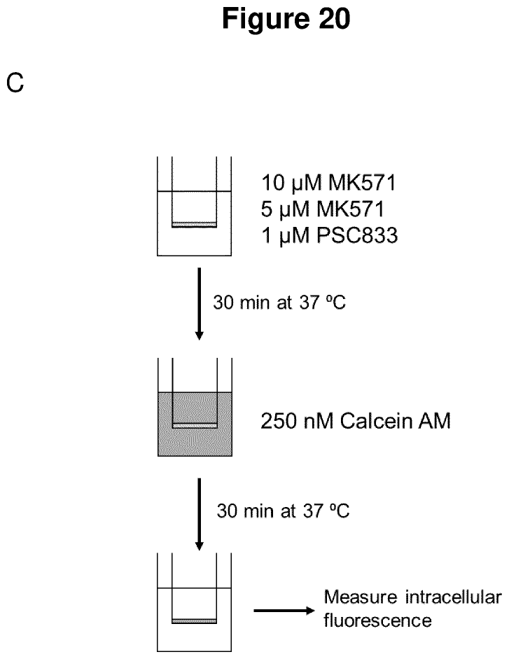

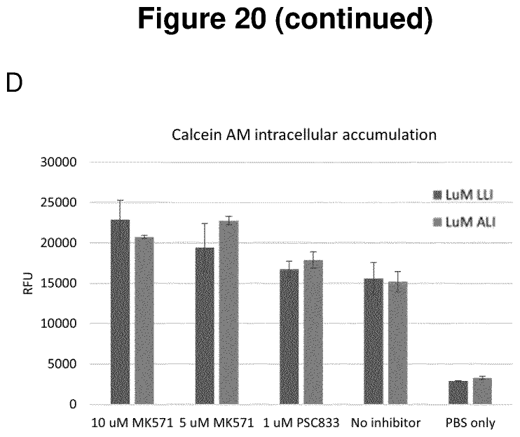

Calcein transport from the basolateral compartment into lung monolayers grown in either the LuM LLI or LuM ALI conditions as described in Example 7 was measured on day 16 after seeding. For ALI cultures, the cells were shifted to the ALI culture format 4 days after seeding. The cells were cultured as follows using a specific MRP1 transporter inhibitor (MK571) and a specific P-gp inhibitor (PSC833), in the ‘accumulation’ assay format (FIG. 20A-C).
1. Remove media and rinse cells using PBS.
2. Add fresh buffer under one of the following conditions (37° C.), in both the apical and basolateral compartments in line with expected expression of MRP transporters:
   1. 10 μM MK571, 30 mins,
   2. 5 μM MK571, 30 mins,
   3. 1 μM PSC-833, 30 mins, or
   4. Untreated Control, 30 mins.
3. Add Calcein AM, final concentration 250 nM, to the basolateral compartment and incubate for 30 minutes at 37° C.
4. Analyse cellular accumulation of Calcein AM using fluorescence (Excitation λ 490 nm, Emission λ 520 nm).
5. Assess accumulation differences.

Increased cellular accumulation of Calcein AM was observed in the presence of both MRP1 and P-gp inhibitor (FIG. 20D), for both LuM LLI and LuM ALI culture conditions.

### Calcein Transport Assay—Pulse-Chase Assay

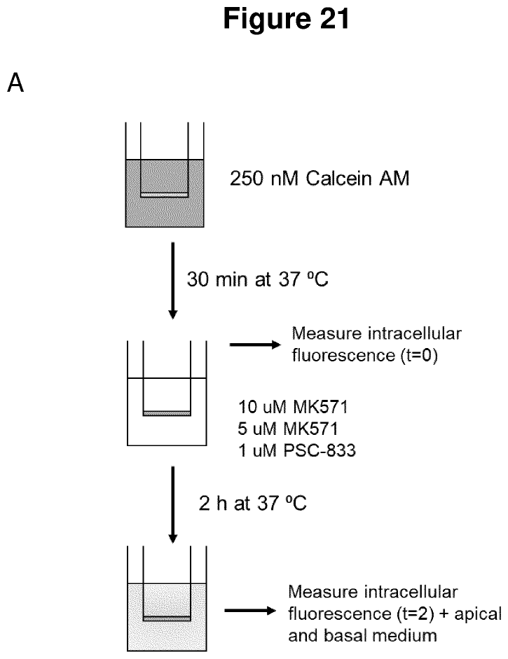

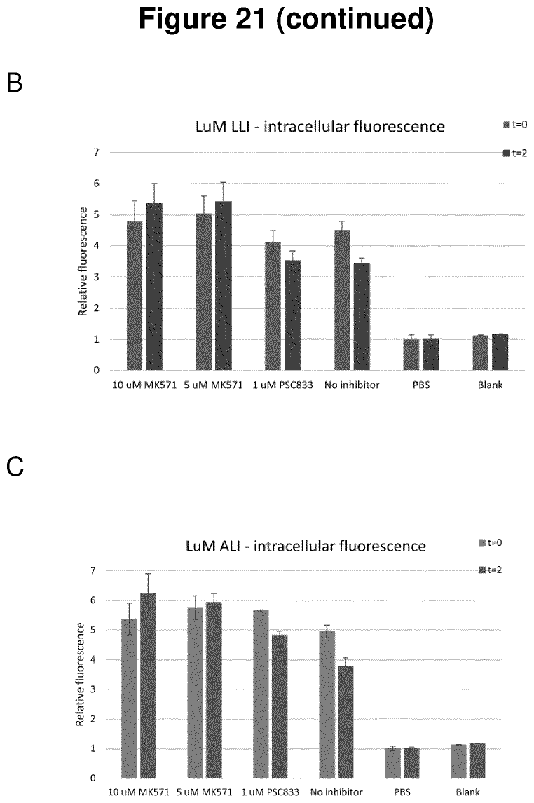

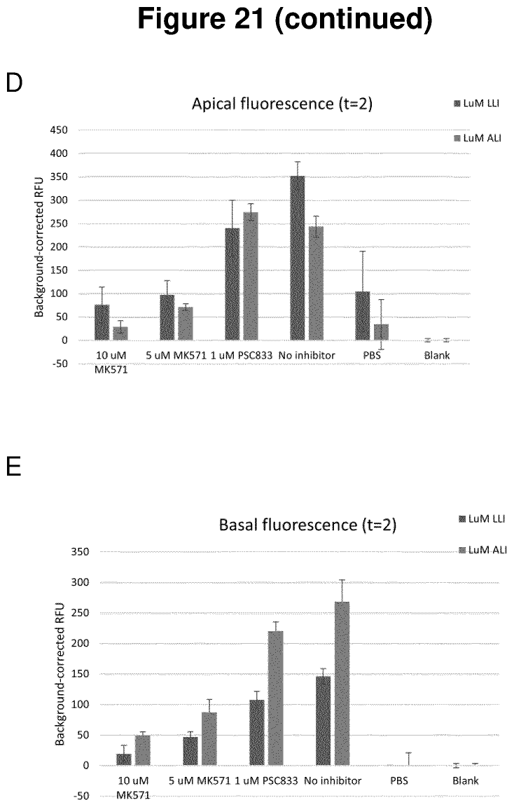

A Calcein AM transport assay was performed in similar conditions to the ‘accumulation’ format using the lung-C monolayer culture, except for the following modifications: monolayers were exposed to 250 nM Calcein AM for 30 minutes at 30° C. After washing with PBS, the baseline intracellular fluorescence was measured (T=0). Monolayers were then further incubated with or without inhibitors (MK571 or PSC-833) in PBS for a further 2 hours at 37° C. The intracellular fluorescence and the fluorescence in the apical and basal medium was measured at T=2 hours (FIG. 21A).

In both the LLI and ALI formats, inhibition of MRP1 increases the intracellular accumulation of Calcein AM (FIGS. 21B and C). After the Calcein AM is removed from the media in the apical and/or basolateral compartments, inhibition of MRP1 results in decreased efflux of Calcein AM (FIGS. 21D and E) in both LLI and ALI formats which is in line with the consistent expression of MRP1 across different culture conditions. P-gp inhibition did not show notable effects on Calcein AM accumulation or efflux.

### Example 9. Establishment and Characterisation of Kidney Organoid Monolayers

### Monolayer Establishment and Differentiation

Human kidney organoids from three different donors (kidney-A, kidney-B and kidney-C) were passaged at high density (ratio ˜1:2) three to four days prior to monolayer preparation. On the day of harvesting, medium from the well was used to break the 30 organoid drops and organoids were washed once in DMEM supplemented with 0.1% BSA and Pen/Strep, centrifuged at 450×g for 5 minutes at 8° C., and washed once in PBS without Mg²⁺ and Ca²⁺. Organoids were digested to single cells and small clumps (2-4 cells) using Accutase by incubating in the water bath and checking and resuspending the material every 5 minutes. Single cells were washed with Advanced DMEM/F12, supplemented with 2 mM GlutaMax, 10 mM HEPES and Pen/Strep, centrifuged at 450×g for 5 minutes and 8° C. twice. Cells were passed through a pre-wetted 40 μm cell strainer and resuspended in kidney expansion medium (ADMEM/F12, 1% HEPES, 1% GlutaMAX, 1% penicillin/streptomycin, 1.5% B27 supplement, 10% Rspol-conditioned medium, 50 ng/ml EGF, 100 ng/ml FGF-10, 10 μM Rho-kinase inhibitor Y-27632, 5 μM A8301, 0.1 mg/ml Primocin) with a density of 2 million cells/ml supplemented with 10 μM RhoKI. In parallel with organoid preparation, Corning® HTS Transwell® 96 well permeable supports, polyester membrane with 0.4 μm pore size inserts were placed in the corresponding receiver plate. Matrigel was diluted 40× with ice-cold PBS (with Ca2+ and Mg2+). Apical surfaces of transwells were either left uncoated, or coated by applying 65 μl of 2.5% Matrigel for 1 hour at 37° C. After carefully removing PBS from the coated inserts, 300 μl of kidney expansion medium was added to the basolateral compartment. Transwells were seeded by adding 100 μl of cell suspension at various cell densities (30,000-250,000 cells/transwell) on the apical compartment. Plates were incubated at 37° C. and 5% CO₂ and medium was refreshed three times a week.

Coating of the transwells with Matrigel, and seeding a higher number of cells up resulted in higher TEER values in the monolayers (FIG. 22B), with no further benefit apparent with seeding densities above 100,000 cells/transwell (FIG. 22A). Therefore, Matrigel coating and 100,000 cells/transwell were used for all subsequent experiments.

The following culture conditions were assessed: culture in kidney expansion medium (KEM) throughout, addition of 1 uM decitabine to the kidney expansion medium on day 2 after seeding (DAC), and change of the medium to kidney differentiation medium (ADMEM/F12, 1% HEPES, 1% GlutaMAX, 1% penicillin/streptomycin) on day 3 after seeding (KDM). The kidney expansion and differentiation media have previously been described in Schutgens et al. (Nature Biotechnology 37: 303-313, 2019). Decitabine is a DNA methyltransferase inhibitor, and the inventors hypothesized that its addition may enhance expression of transporter proteins.

The morphology, barrier function, marker expression (the present example) and transport function (Example 10) of the kidney monolayers were assessed on day 7 after seeding.

### Morphology

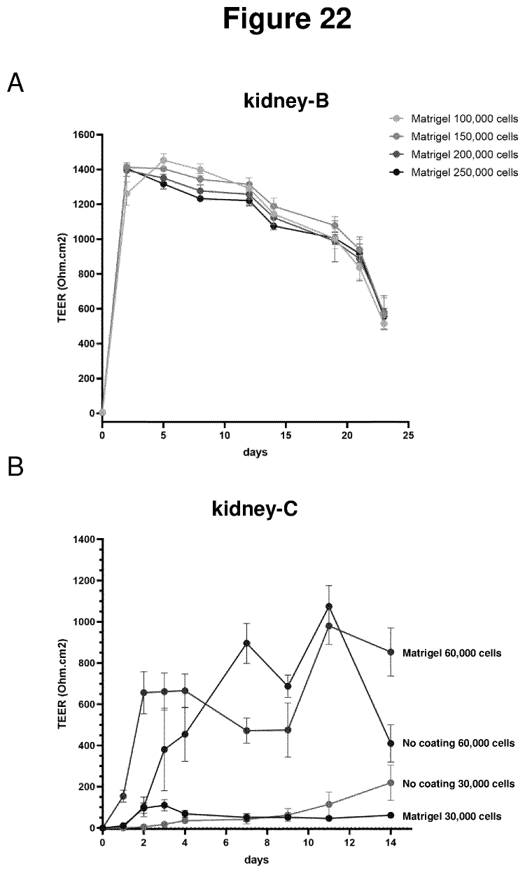

The morphology of the kidney organoid monolayers was assessed using H&E staining, which revealed a very thin layer of cells, with a mixture of different cell types (FIG. 23A-C), including ciliated cells in the kidney-C-derived monolayer grown in KDM (FIG. 23C).

### Permeability

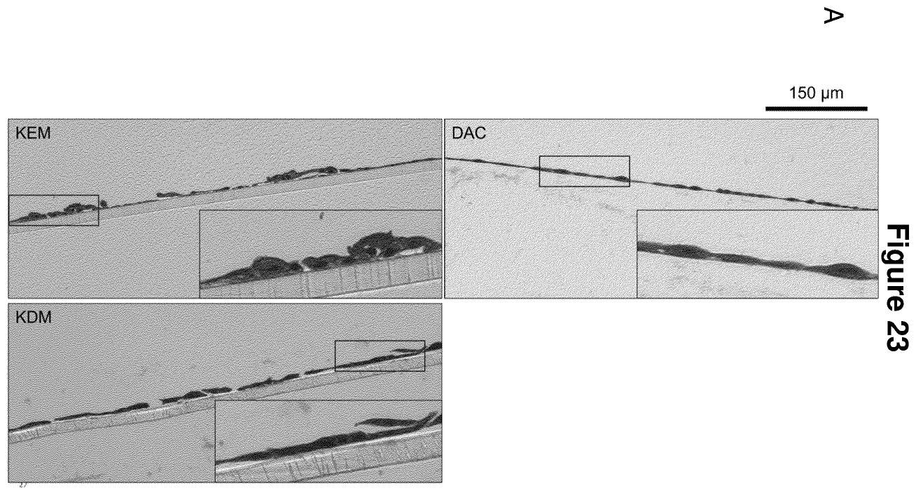

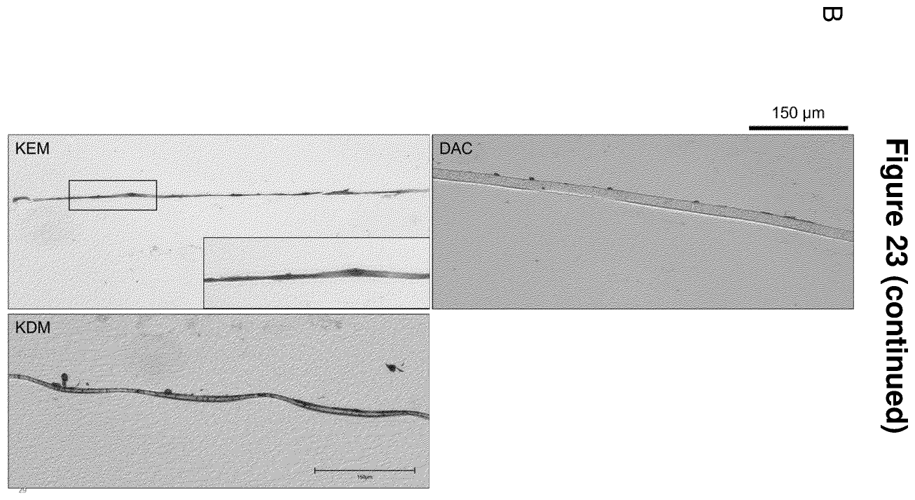

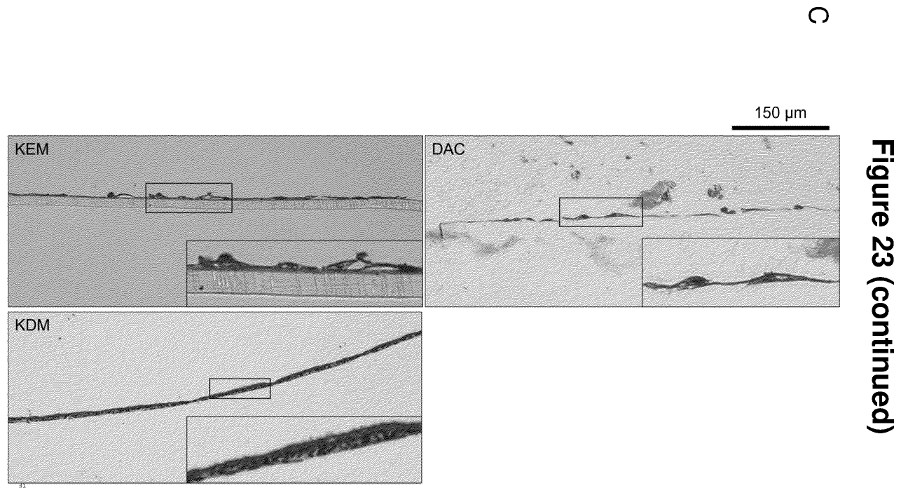

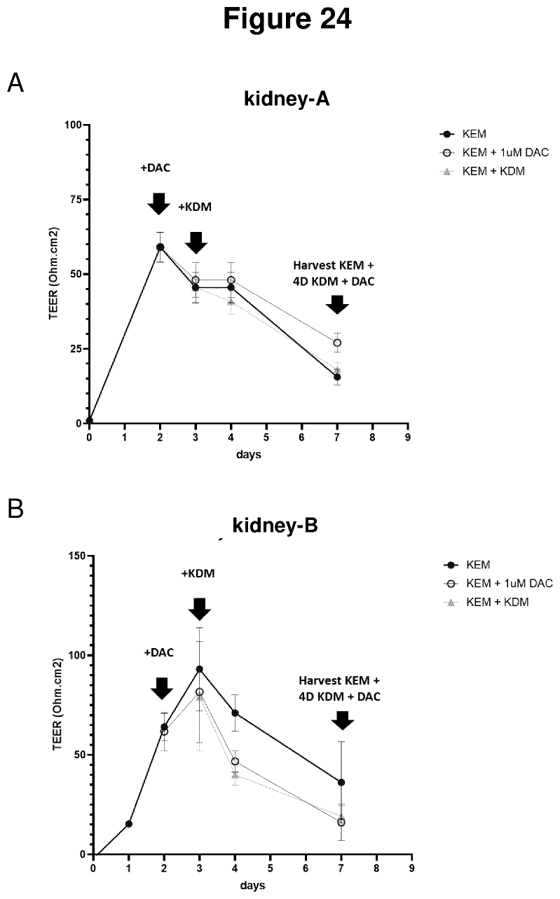

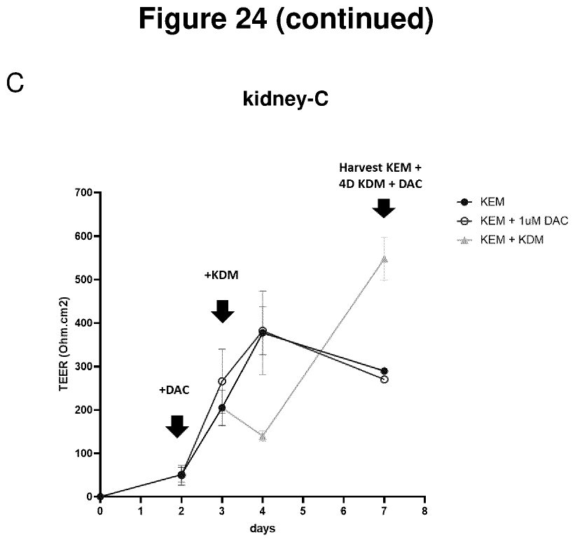

Permeability of the monolayers was assessed throughout the experiment by measuring TEER (FIG. 24A-C).

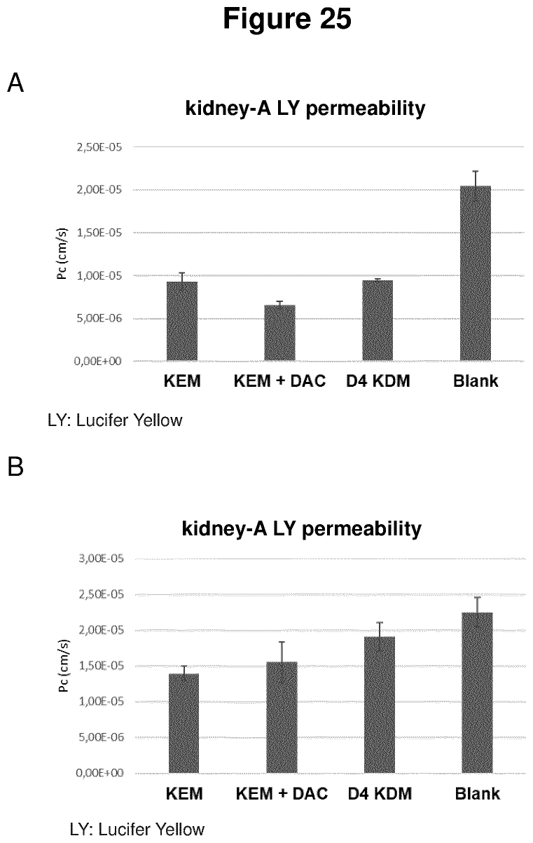

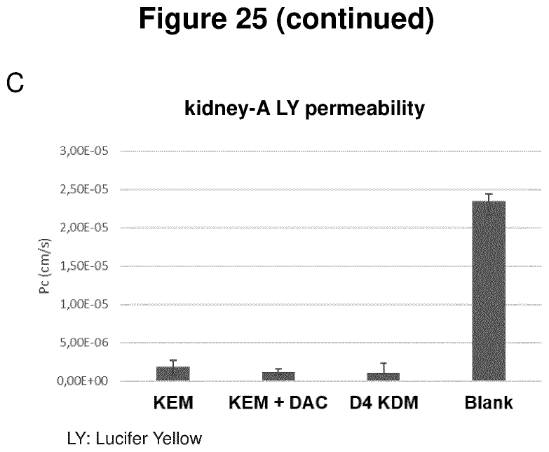

Permeability was also assessed using the lucifer yellow assay. Briefly, 60 μM lucifer yellow was added to the apical compartment. After a 60-minute incubation at 37° C., diffusion of lucifer yellow into the basolateral compartments was measured. The results are shown in FIG. 25A-C.

### Marker Expression

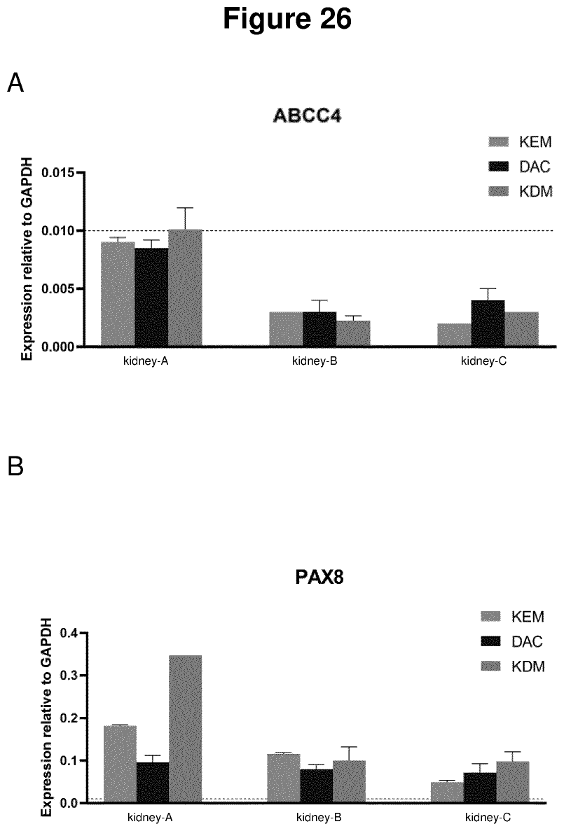

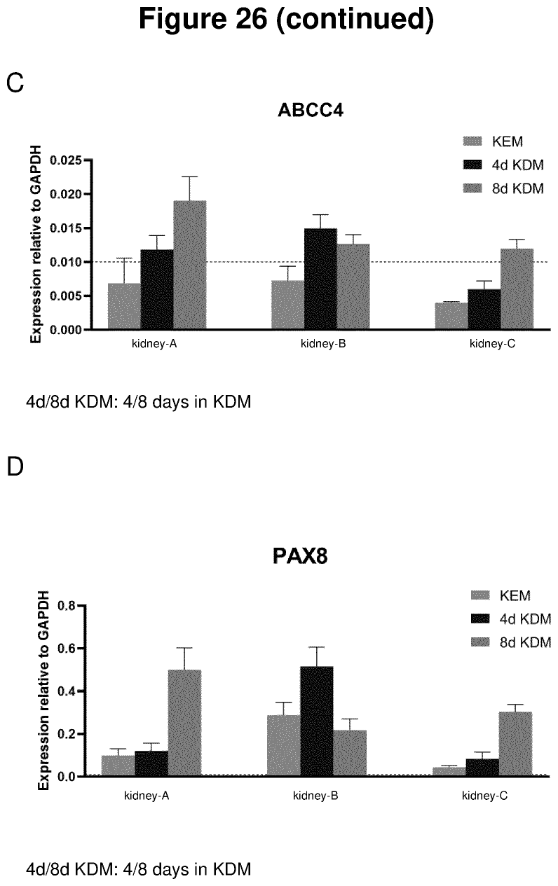

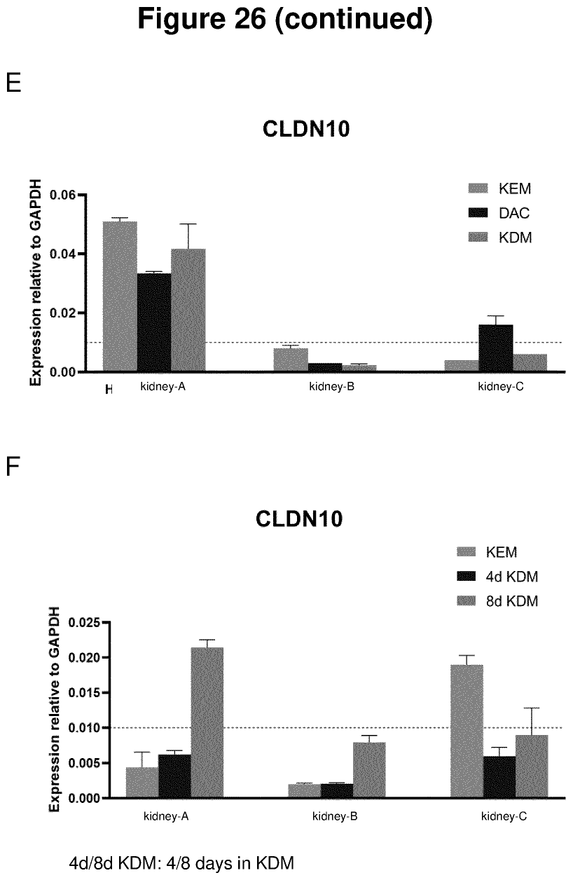

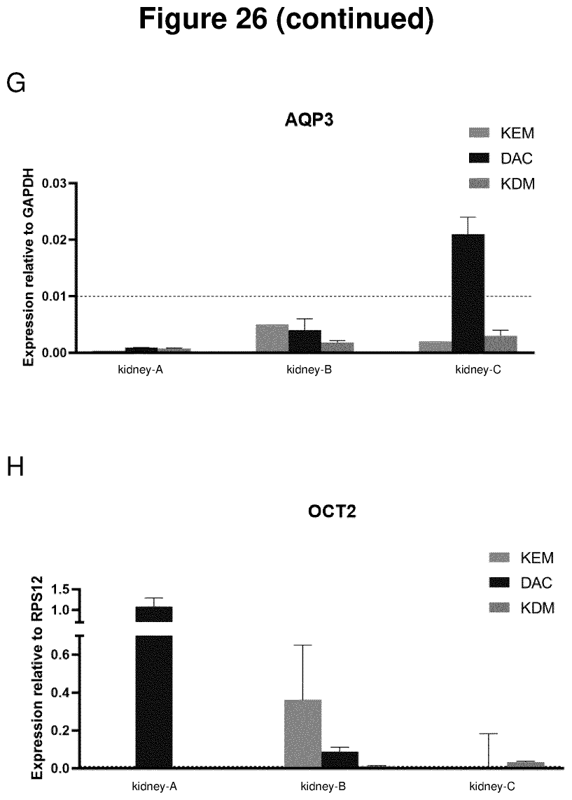

Expression of various kidney markers and transporter proteins in the monolayers, was measured using RT-qPCR. Expression was also assessed in the organoids which were used for seeding the monolayers. The organoids were grown in KEM, or were differentiated in KDM for 4 or 8 days. Expression of the following kidney markers was assessed: ABCC4 (proximal tubule marker), PAX8 (kidney epithelial marker), CLDN10 (loop of Henle marker), SLC12A3 (distal tubule marker) and AQP3 (collecting duct marker). Expression of the transporter proteins OAT1, OAT3, OCT2, MATE1 and MATE2-K was also measured. The results are shown in FIG. 26.

The expression levels of SLC12A3 were below the threshold in organoids and organoid-derived monolayers (not shown). The same was true for AQP3 in organoids (not shown), but expression was detected in one of the monolayers (FIG. 26G). The expression of OAT1 and OAT3 was not detected in either organoids or organoid-derived monolayers (not shown).

In summary, the kidney monolayers were found to display a heterogenous cell composition, which was also reflected in their barrier properties as shown using TEER and the lucifer yellow assay (FIGS. 24 and 25). The same kidney markers that are expressed in organoids were also expressed in monolayers at similar levels, with the exception of CLDN10, which was more highly expressed in the kidney-A monolayers relative to the kidney-A organoids. The monolayers also expressed three out of the five assessed transporters, the same ones that are expressed in organoids, but at different levels. KDM was not found to further increase transporter expression in monolayers.

### Example 10. Transport Assays in Kidney Organoid Monolayers

This Example demonstrates the development transport assays that allow measurement of transporter function of kidney organoid monolayers through accumulation of fluorescent dyes in the epithelium. The organoid line kidney-C was selected for the transport assays, on account of its tight barrier function (FIGS. 24C and 25C).

### Calcein Transport Assay

Calcein transport from the basolateral compartment into kidney monolayers grown in either the KEM or DAC conditions as described in Example 9 was measured on day 7 after seeding in the presence or absence of the P-gp inhibitor PSC-833 as follows:
6. Remove media and rinse cells using PBS.
7. Add fresh buffer under one of the following conditions (37° C.):
   1. 1 μM PSC-833, 30 mins, or
   2. Untreated Control, 30 mins.
8. Add Calcein AM, final concentration 250 nM, to the basolateral compartment and incubate for 30 minutes at 37° C.
9. Analyse cellular accumulation of Calcein AM using fluorescence (Excitation λ 490 nm, Emission λ 520 nm).
10. Assess accumulation differences.

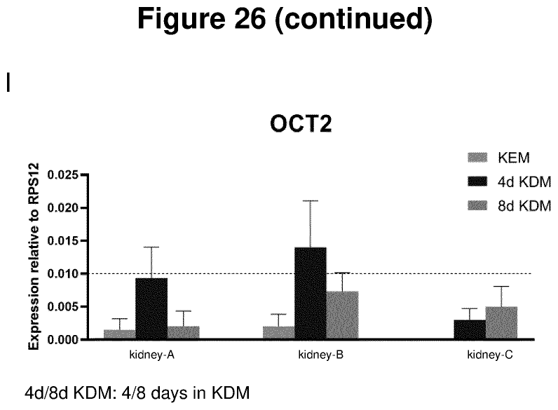

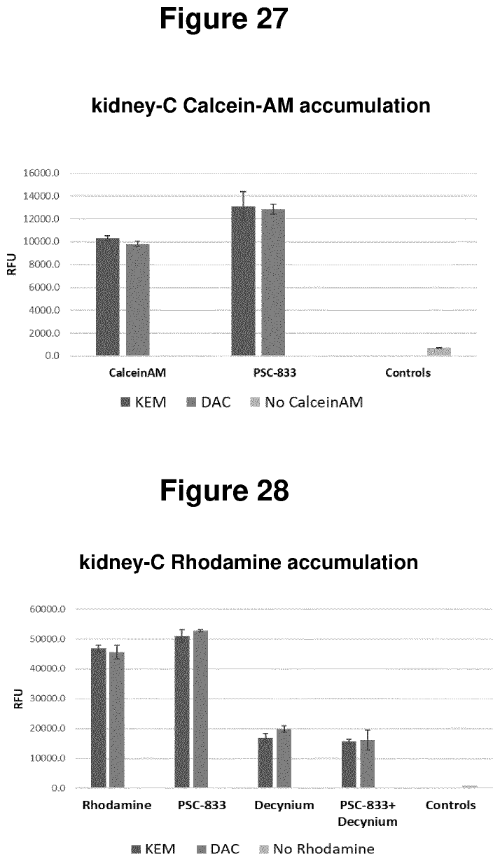

Increased cellular accumulation of Calcein AM was observed in the presence of P-gp inhibitor (FIG. 27).

### Rhodamine Transport Assay

Rhodamine transport from the basolateral compartment into kidney monolayers grown in either the KEM or DAC conditions as described in Example 9 was measured on day 7 after seeding in the presence or absence of the P-gp inhibitor PSC-833 and/or OCT2 inhibitor Decynium-22 as follows:
1. Remove media and rinse cells using PBS.
2. Add fresh buffer under one of the following conditions (37° C.):
   1. 1 μM Decynium-22, 30 mins,
   2. 1 μM PSC-833, 30 mins,
   3. 1 μM Decynium-22 and 1 μM PSC-833, 30 mins, or
   4. Untreated Control, 30 mins.
3. Add Rhodamine 123 at a final concentration 250 nM to the basolateral compartment and incubate for 30 minutes at 37° C.
4. Analyse cellular accumulation of Rhodamine 123 using fluorescence (Excitation λ 490 nm, Emission λ 520 nm).
5. Assess accumulation differences.

Increased cellular accumulation of Rhodamine was observed in the presence of P-gp inhibitor, and decreased accumulation was observed with the OCT2 inhibitor. DAC treatment did not result in increased Rhodamine 123 loading/transport (FIG. 28).

1. A method of obtaining an intestinal organoid-derived monolayer comprising:
i. digesting or dissociating one or more intestinal organoids into a suspension of single cells and/or organoid fragments;
ii. seeding a semi-permeable membrane with said suspension;
iii. culturing the cells and/or organoid fragments in the presence of an expansion medium until a monolayer is formed; and
iv. culturing the monolayer in the presence of a differentiation medium comprising a Notch inhibitor, an EGFR pathway inhibitor and a Wnt agonist.

2. The method of claim 1, wherein the monolayer is cultured in the presence of an expansion medium until it reaches transepithelial electrical resistance (TEER) of about 100 Ω·cm².

3. The method of claim 1 or claim 2, wherein TEER of the monolayer further increases during the step of culturing the monolayer in the presence of the differentiation medium.

4. The method of claim 3, wherein TEER of the monolayer reaches more than 500, more than 600, more than 700, more than 800, more than 900, more than 1000, more than 1100, more than 1200, more than 1300, more than 1400 or more than 1500 Ω·cm² during the step of culturing the monolayer in the presence of the differentiation medium.

5. The method of any one of the preceding claims, wherein the expansion medium comprises a receptor tyrosine kinase ligand, a BMP inhibitor and a Wnt agonist and, optionally, nicotinamide and a p38 MAPK inhibitor, such as SB202190.

6. The method of any one of claims 1-5, wherein the receptor tyrosine kinase ligand is a ligand for RTK class I (EGF receptor family) (ErbB family), a ligand for RTK class II (Insulin receptor family), a ligand for RTK class IV (FGF receptor family) or a ligand for RTK class VI (HGF receptor family).

7. The method of claim 6, wherein the receptor tyrosine kinase ligand is selected from the group consisting of: epidermal growth factor (EGF), neuregulin, fibroblast growth factor (FGF), hepatocyte growth factor (HGF) and insulin-like growth factor (IGF).

8. The method of any one of claims 5-7, wherein the BMP inhibitor is selected from the group consisting of noggin, sclerostin, chordin, CTGF, follistatin, gremlin, tsg, sog, LDN193189 or dorsomorphin.

9. The method of any one of claims 1-8, wherein the Wnt agonist is selected from the group consisting of: Rspondin, Wnt conditioned medium and Wnt surrogate.

10. The method of any one of claims 1-9, wherein the Notch inhibitor is a gamma secretase inhibitor, optionally selected from the group consisting of: DAPT, dibenzazepine (DBZ), benzodiazepine (BZ) and LY-411575.

11. The method of any one of claims 1-10, wherein the EGFR pathway inhibitor is selected from: (1) an EGFR inhibitor, such as Gefitinib, (2) an EGFR and ErbB2 inhibitor, such as Afatinib, (3) an inhibitor of the RAS-RAF-MAPK pathway, (4) an inhibitor of the PI3K/AKT pathway and (5) an inhibitor of the JAK/STAT pathway.

12. The method of claim 11, wherein the EGFR pathway inhibitor is an inhibitor of the RAS-RAF-MAPK pathway, e.g. a MEK inhibitor, such as PD0325901.

13. The method of any one of the preceding claims, wherein:
i. the monolayer is cultured in the presence of an expansion medium for at least 2 days, at least 3 days, at least 4 days, at least 5 days, at least 6 days, at least 7 days, at least 8 days, at least 9 days or at least 10 days, preferably wherein the monolayer is cultured in the presence of an expansion medium for 3-9 days; and/or
ii. the monolayer is cultured in the presence of the differentiation medium for at least 3 days, at least 4 days, at least 5 days, at least 6 days, at least 7 days, at least 8 days, at least 9 or more, preferably wherein the monolayer is cultured in the presence of a differentiation medium for 4-8 days.

14. The method of any one of the preceding claims, wherein the monolayer is cultured in the presence of an extracellular matrix.

15. An intestinal organoid-derived monolayer obtainable or obtained by the method of any one of claims 1-14.

16. The method or organoid-derived monolayer of any one of the preceding claims, wherein the monolayer comprises one or more of the following cell types: Lgr5+ stem cell, enterocyte, goblet cell, Paneth cell and enteroendocrine cell.

17. The method or organoid-derived monolayer of any one of the preceding claims, wherein the monolayer is derived from a mammal.

18. The method or organoid-derived monolayer of claim 17, wherein the monolayer is derived from a human.

19. The method or organoid-derived monolayer of claim 19, wherein the human has a disease or disorder of the digestive system, such as inflammatory bowel disease (e.g. Crohn's disease or ulcerative colitis), coeliac disease or leaky gut syndrome.

20. A method of obtaining a lung organoid-derived monolayer comprising:
i. digesting or dissociating one or more lung organoids into a suspension of single cells and/or organoid fragments;
ii. seeding a semi-permeable membrane with said suspension; and
iii. culturing the cells and/or organoid fragments in the presence of an expansion medium until a monolayer is formed.

21. The method of claim 20, wherein the method further comprises:
iv. culturing the monolayer in the presence of a differentiation medium.

22. The method of claim 20 or claim 21, wherein the monolayer is cultured in the presence of an extracellular matrix.

23. The method of any one of claims 20-22, wherein the expansion medium comprises one or more receptor tyrosine ligands, a Wnt agonist, a TGF-beta inhibitor a BMP inhibitor and, optionally, a Rho kinase inhibitor, such as Y-27632, and/or a p38 MAPK inhibitor, such as SB202190.

24. The method of any one of claims 20-23, wherein the differentiation medium comprises one or more receptor tyrosine kinases, a Wnt agonist, a Notch inhibitor, a BMP pathway activator and, optionally, a Rho kinase inhibitor, such as Y-27632, and/or a p38 MAPK inhibitor, such as SB202190.

25. The method of claim 23 or claim 24, wherein the receptor tyrosine kinase ligand is a ligand for RTK class I (EGF receptor family) (ErbB family), a ligand for RTK class II (Insulin receptor family), a ligand for RTK class IV (FGF receptor family) or a ligand for RTK class VI (HGF receptor family).

26. The method of claim 25, wherein the receptor tyrosine kinase ligand is selected from the group consisting of: epidermal growth factor (EGF), neuregulin, fibroblast growth factor (FGF), hepatocyte growth factor (HGF) and insulin-like growth factor (IGF).

27. The method of any one of claims 23-26, wherein the BMP inhibitor is selected from the group consisting of noggin, sclerostin, chordin, CTGF, follistatin, gremlin, tsg, sog, LDN193189 or dorsomorphin.

28. The method of any one of claims 23-27, wherein the Wnt agonist is selected from the group consisting of: Rspondin, Wnt conditioned medium and Wnt surrogate.

29. The method of any one of claims 23-28, wherein the TGF-beta inhibitor is selected from the group consisting of: A83-01, SB-431542, SB-505124, SB-525334, LY 364947, SD-208 and SJN 2511.

30. The method of any one of claims 24-29, wherein the Notch inhibitor is a gamma secretase inhibitor, optionally selected from the group consisting of: DAPT, dibenzazepine (DBZ), benzodiazepine (BZ) and LY-411575.

31. The method of any one of claims 24-30, wherein the BMP pathway activator is selected from the group consisting of BMP7, BMP4 and BMP2.

32. The method of any one of claims 20-31, wherein the method comprises seeding the semi-permeable membrane with less than about 20,000 cells, less than about 30,000 cells, less than about 40,000 cells, less than about 50,000 cells, less than about 60,000 cells, less than about 70,000 cells, less than about 80,000 cells, less than about 90,000 cells, less than about 100,000 cells, or less than about 250,000 cells, for example, in a standard 96-well format.

33. The method of any one of claims 20-32, wherein the method comprises seeding the semi-permeable membrane with about 30,000 cells, about 40,000 cells, about 50,000 cells, about 60,000 cells, about 70,000 cells, about 80,000 cells or about 90,000 cells, for example, in a standard 96-well format.

34. The method of any one of claims 20-33, wherein the method comprises seeding the semi-permeable membrane with about 5,000-500,000 cells, about 10,000-250,000 cells, about 20,000-100,000 cells, about 30,000-50,000 cells, about 35,000-45,000 cells, or preferably about 40,000 cells, for example, in a standard 96-well format.

35. The method of any one of claims 20-34, wherein the method comprises adjusting the suspension of single cells and/or organoid fragments to less than about 0.2×10⁶ cells per mL, less than about 0.3×10⁶ cells per mL, less than about 0.4×10⁶ cells per mL, less than about 0.5×10⁶ cells per mL, less than about 10⁶ cells per mL, less than about 2×10⁶ cells per mL, less than about 3×10⁶ cells per mL, less than about 4×10⁶ cells per mL or less than about 5×10⁶ cells per mL before seeding.

36. The method of any one of claims 20-35, wherein the method comprises adjusting the suspension of single cells and/or organoid fragments to about 0.2×10⁶ cells per mL, about 0.3×10⁶ cells per mL, about 0.4×10⁶ cells per mL, about 0.5×10⁶ cells per mL, about 10⁶ cells per mL, about 2×10⁶ cells per mL, about 3×10⁶ cells per mL, about 4×10⁶ cells per mL or about 5×10⁶ cells per mL before seeding.

37. The method of any one of claims 20-36, wherein the method comprises adjusting the suspension of single cells and/or organoid fragments to about 0.1-1×10⁶ cells per mL, about 0.25-0.75×10⁶ cells per mL, about 0.3-0.5×10⁶ cells per mL, about 0.35-0.45×10⁶ cells per mL, preferably about 0.4×10⁶ cells per mL before seeding.

38. The method of any one of claims 20-37, wherein:
i. the monolayer is cultured in the presence of an expansion medium for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days or more, preferably wherein the monolayer is cultured in the presence of an expansion medium for 3-8 days; and/or
ii. the monolayer is cultured in the presence of a differentiation medium for at least 3 days, at least 4 days, at least 5 days, at least 6 days, at least 7 days, at least 8 days, at least 9 or more, preferably wherein the monolayer is cultured in the presence of a differentiation medium for 8 days.

39. The method of any one of claims 20-38, wherein the method further comprises removing the expansion or differentiation medium from the apical compartment.

40. The method of claim 39, wherein the medium is removed from the apical compartment 10-16 days after seeding, for example, 11 days, 12 days, 13 days, 14 days or 15 days, preferably 13 days, after seeding.

41. A lung organoid-derived monolayer obtained or obtainable by the method of any one of claims 20-40.

42. The method or organoid-derived monolayer of any one of claims 20-41, wherein the monolayer comprises one or more of the following cell types: club cells, basal cells, ciliated cells, goblet cells, alveolar type I cells and alveolar type II cells.

43. The method or organoid-derived monolayer of any one of claims 20-42, wherein the monolayer is derived from a mammal, for example a human.

44. A method of obtaining a kidney organoid-derived monolayer comprising:
i. digesting or dissociating one or more kidney organoids into a suspension of single cells and/or organoid fragments;
ii. seeding a semi-permeable membrane with said suspension; and
iii. culturing the cells and/or organoid fragments in the presence of an expansion medium until a monolayer is formed.

45. The method of claim 44, wherein the method further comprises:
iv. culturing the monolayer in the presence of a differentiation medium.

46. The method of claim 44 or claim 45, wherein the monolayer is cultured in the presence of an extracellular matrix.

47. The method of any one of claims 44-46, wherein the expansion medium comprises one or more receptor tyrosine ligands, a Wnt agonist, and a TGF-beta inhibitor and, optionally, a Rho kinase inhibitor.

48. The method of any one of claims 44-47, wherein the method comprises seeding the semi-permeable membrane with less than about 100,000 cells, less than about 150,000 cells, less than about 200,000 cells, or less than about 250,000 cells, for example, in a standard 96-well format.

49. The method of any one of claims 44-48, wherein the method comprises seeding the semi-permeable membrane with about 30,000 cells, about 40,000 cells, about 50,000 cells, about 60,000 cells, about 70,000 cells, about 80,000 cells, about 90,000 cells, or about 100,000 cells, for example, in a standard 96-well format.

50. The method of any one of claims 44-49, wherein the method comprises seeding the semi-permeable membrane with about 20,000-500,000 cells, about 30,000-400,000 cells, about 40,000-300,000 cells, about 50,000-250,000 cells, about 60,000-200,000 cells, about 70,000-150,000 cells, about 80,000-120,000 cells, or preferably about 100,000 cells, for example, in a standard 96-well format.

51. The method of any one of claims 44-50, wherein the method comprises adjusting the suspension of single cells and/or organoid fragments to less than about 0.5×10⁶ cells per mL, less than about 0.6×10⁶ cells per mL, less than about 0.7×10⁶ cells per mL, less than about 0.8×10⁶ cells per mL, less than about 0.9×10⁶ cells per mL, less than about 10⁶ cells per mL, less than about 1.1×10⁶ cells per mL, less than about 1.2×10⁶ cells per mL, less than about 1.3×10⁶ cells per mL, less than about 1.4×10⁶ cells per mL or less than about 1.5×10⁶ cells per mL before seeding.

52. The method of any one of claims 44-51, wherein the method comprises adjusting the suspension of single cells and/or organoid fragments to about 0.2×10⁶ cells per mL, about 0.3×10⁶ cells per mL, about 0.4×10⁶ cells per mL, about 0.5×10⁶ cells per mL, about 10⁶ cells per mL, about 1.5×10⁶ cells per mL, about 2×10⁶ cells per mL, about 3×10⁶ cells per mL, about 4×10⁶ cells per mL or about 5×10⁶ cells per mL, preferably about 10⁶ cells per mL, before seeding.

53. The method of any one of claims 44-52, wherein the method comprises adjusting the suspension of single cells and/or organoid fragments to about 0.1-5×10⁶ cells per mL, about 0.25-2.5×10⁶ cells per mL, about 0.5-1.5×10⁶ cells per mL, about 0.75-1.25×10⁶ cells per mL, about 0.8-1.2×10⁶ cells per mL, preferably about 10⁶ cells per mL, before seeding.

54. The method of any one of claims 44-53, wherein:
i. the monolayer is cultured in the presence of an expansion medium for at least 1 day, at least 2 days, at least 3 days, at least 4 days, at least 5 days or more, preferably wherein the monolayer is cultured in the presence of an expansion medium for 1-3 days, more preferably 2 days; and/or
ii. the monolayer is cultured in the presence of a differentiation medium for at least 2 days, at least 3 days, at least 4 days, at least 5 days, at least 6 days, at least 7 days or more, preferably wherein the monolayer is cultured in the presence of a differentiation medium for 3-5 days, more preferably 4 days.

55. The method of any one of claims 44-54, wherein the method further comprises adding a histone deacetylase inhibitor, such as decitabine, to the expansion or differentiation medium.

56. The method of claim 55, wherein the histone deacetylase inhibitor is added 1-3 days after seeding, preferably 2 days after seeding.

57. A kidney organoid-derived monolayer obtained or obtainable by the method of any one of claims 44-56.

58. The kidney organoid-derived monolayer of claim 57, wherein the monolayer has TEER of more than 25, more than 50, more than 75, more than 100, more than 200, more than 300, more than 400, more than 500, more than 600, more than 700, more than 800, more than 900, more than 1000, more than 1100, more than 1200, more than 1300 or more than 1400 Ω·cm².

59. The method or organoid-derived monolayer of any one of claims 44-58, wherein the monolayer comprises one or more of the following cell types: proximal tubule cells, kidney epithelial cells, loop of Henle cells, distal tubule cells and collecting duct cells.

60. The method or organoid-derived monolayer of any one of claims 44-59, wherein the monolayer is derived from a mammal, for example a human.

61. Use of an organoid-derived monolayer according to any one of claims 15-19, 41-43 and 57-60 in an assay assessing epithelial viability, metabolic activity, permeability, barrier function integrity and/or activity of transporter proteins.

62. A method of identifying a compound capable of modulating epithelial viability, metabolic activity, permeability, barrier function integrity and/or activity of transporter proteins comprising:
i. contacting an organoid-derived monolayer, for example according to any one of claims 15-19, 41-43 and 57-60, with one or more candidate molecules; and
ii. assessing the viability, metabolic activity, permeability and/or barrier function integrity of the organoid-derived monolayer and/or activity of transporter proteins in the organoid-derived monolayer.

63. A method of assessing the effect of a compound on epithelial viability, metabolic activity, permeability, barrier function integrity and/or activity of transporter proteins comprising:
i. contacting an organoid-derived monolayer, for example according to any one of claims 15-19, 41-43 and 57-60, with said compound; and
ii. assessing the viability, metabolic activity, permeability and/or barrier function integrity of the organoid-derived monolayer and/or activity of transporter proteins in the organoid-derived monolayer.

64. The method of claim 62 or claim 63, wherein the method further comprises contacting the organoid-derived monolayer with one or more proinflammatory cytokines.

65. The method of claim 64, wherein the one or more proinflammatory cytokines are selected from the group consisting of: IFN-γ, TNF-α and IL-1α.

66. A method of identifying a mutation associated with epithelial viability, metabolic activity, permeability, barrier function integrity and/or activity of transporter proteins comprising:
i. assessing the viability, metabolic activity, permeability and/or barrier function integrity of an organoid-derived monolayer and/or activity of transporter proteins in an organoid-derived monolayer, for example an organoid monolayer according to any one of claims 15-19, 41-43 and 57-60; and
ii. determining the presence of one or more mutations in the genome of one or more cells in the organoid-derived monolayer.

67. A method of diagnosing a disease or affliction that affects epithelial viability, metabolic activity, permeability, barrier function integrity and/or activity of transporter proteins, or determining an increased risk of said disease or affliction, in a human subject comprising:
i. obtaining an organoid-derived monolayer from said human subject as described in any one of claims 1-14, 16-40, 42-56 and 58-60; and
ii. testing the viability, metabolic activity, permeability and/or barrier function integrity of the organoid-derived monolayer and/or activity of transporter proteins in the organoid-derived monolayer,
wherein a test result above or below a reference value indicates the presence of, or an increased risk of, said disease or affliction in the human subject.

68. The method of claim 67, wherein the reference value is a value obtained from a control, e.g. an organoid-derived monolayer obtained from a healthy human subject.

69. The method of claim 67 or claim 68, wherein the disease or affliction is a disease or disorder of the digestive system, such as inflammatory bowel disease (e.g. Crohn's disease or ulcerative colitis), coeliac disease or leaky gut syndrome.

70. A method of predicting the likelihood of a patient's response to a candidate compound comprising:
i. obtaining an organoid-derived monolayer from said patient as described in any one of claims 1-14, 16-40, 42-56 and 58-60;
ii. contacting the organoid-derived monolayer with said compound; and
iii. assessing the viability, metabolic activity, permeability and/or barrier function integrity of the organoid-derived monolayer and/or activity of transporter proteins in the organoid-derived monolayer.

71. The use or method of any one of claims 61-70, wherein assessing the barrier function integrity of the organoid-derived monolayer comprises measuring TEER of the organoid-derived monolayer.

72. The use or method of any one of claims 61-71, wherein assessing the permeability of the organoid-derived monolayer comprises measuring the rate of passive diffusion of a reporter compound across the monolayer.

73. The use or method of claim 72, wherein said reporter compound is a dye, optionally a fluorescent dye, such as Lucifer yellow.

74. The use or method of any one of claims 61-73, wherein assessing the activity of transporter proteins comprises measuring the rate of transport of a substrate of a transporter protein across the monolayer, optionally in the presence of an inhibitor of said transporter protein.

75. The use or method of any one of claims 61-74, wherein assessing the activity of transporter proteins comprises measuring the rate of transport of a substrate of a transporter protein into the cells of the monolayer, optionally in the presence of an inhibitor of said transporter protein.

76. The use or method of claim 74 or claim 75, wherein the substrate is a dye, such as Rhodamine 123 or Calcein AM.
